(19) United States
(12) Patent Application Publication
(10) Pub. No.: US 2012/0142705 A1
(43) Pub. Date: Jun. 7, 2012

(54) ISATIN AND OXINDOLE COMPOUNDS

(76) Inventor: Robert Francis Kester, West Orange, NJ (US)

(21) Appl. No.: 13/277,260

(22) Filed: Oct. 20, 2011

Related U.S. Application Data
(60) Provisional application No. 61/419,999, filed on Dec. 6, 2010.

(51) Int. Cl. A61K 31/497; C07D 417/12; A61P 3/10; A61K 31/4155; C07D 401/12; A61K 31/4439; C07D 403/12; A61K 31/427

(52) U.S. Cl. 514/255.05; 544/336; 548/181; 514/371; 548/364.7; 514/407; 546/277.7; 514/339

(57) ABSTRACT

Provided herein are compounds of the formula (I):

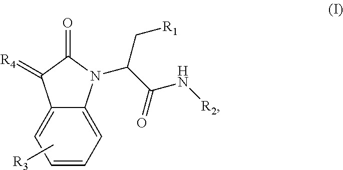

as well as pharmaceutically acceptable salts thereof, wherein the substituents are as those disclosed in the specification. These compounds, and the pharmaceutical compositions containing them, are useful for the treatment of metabolic diseases and disorders such as, for example, type II diabetes mellitus.

### PRIORITY TO RELATED APPLICATION(S)

This application claims the benefit of U.S. Provisional Application No. 61/419,999, filed Dec. 6, 2010, which is hereby incorporated by reference in its entirety.

### FIELD OF THE INVENTION

The present invention relates to organic compounds useful for therapy and/or prophylaxis in a mammal, and in particular to activators of glucokinase useful for treating metabolic diseases and disorders.

### BACKGROUND OF THE INVENTION

Glucokinase (GK) is one of four hexokinases that are found in mammals (Colowick, S. P., in The Enzymes, Vol. 9 (P. Boyer, ed). Academic Press, New York, N.Y., pages 1-48, 1973). The hexokinases catalyze the first step in the metabolism of glucose, i.e., the conversion of glucose to glucose-6-phosphate. Glucokinase has a limited cellular distribution, being found principally in pancreatic β-cells and liver parenchymal cells. In addition, GK is a rate-controlling enzyme for glucose metabolism in these two cell types that are known to play critical roles in whole-body glucose homeostasis (Chipkin, S. R., Kelly, K. L., and Ruderman, N. B. in Joslin's Diabetes (C. R. Khan and G. C. Wier, eds)., Lea and Febiger, Philadelphia, Pa., pages 97-115, 1994). The concentration of glucose at which GK demonstrates half-maximal activity is approximately 8 mM. The other three hexokinases are saturated with glucose at much lower concentrations (<1 mM). Therefore, the flux of glucose through the GK pathway rises as the concentration of glucose in the blood increases from fasting (5 mM) to postprandial (≈10-15 mM) levels following a carbohydrate-containing meal (Printz, R. G., Magnuson, M. A., and Granner, D. K. in Ann. Rev. Nutrition Vol. 13 (R. E. Olson, D. M. Bier, and D. B. McCormick, eds)., Annual Review, Inc., Palo Alto, Calif., pages 463-496, 1993). These findings contributed over a decade ago to the hypothesis that GK functions as a glucose sensor in β-cells and hepatocytes (Meglasson, M. D. and Matschinsky, F. M. Amer. J. Physiol. 246, E1-E13, 1984). In recent years, studies in transgenic animals have confirmed that GK does indeed play a critical role in whole-body glucose homeostasis. Animals that do not express GK die within days of birth with severe diabetes while animals overexpressing GK have improved glucose tolerance (Grupe, A., Hultgren, B., Ryan, A. et al., Cell 83, 69-78, 1995; Ferrie, T., Riu, E., Bosch, F. et al., FASEB J., 10, 1213-1218, 1996). An increase in glucose exposure is coupled through GK in β-cells to increased insulin secretion and in hepatocytes to increased glycogen deposition and perhaps decreased glucose production.

The finding that type II maturity-onset diabetes of the young (MODY-2) is caused by loss of function mutations in the GK gene suggests that GK also functions as a glucose sensor in humans (Liang, Y., Kesavan, P., Wang, L. et al., Biochem. J. 309, 167-173, 1995). Additional evidence supporting an important role for GK in the regulation of glucose metabolism in humans was provided by the identification of patients that express a mutant form of GK with increased enzymatic activity. These patients exhibit a fasting hypoglycemia associated with an inappropriately elevated level of plasma insulin (Glaser, B., Kesavan, P., Heyman, M. et al., New England J. Med. 338, 226-230, 1998). While mutations of the GK gene are not found in the majority of patients with type II diabetes, compounds that activate GK and, thereby, increase the sensitivity of the GK sensor system will still be useful in the treatment of the hyperglycemia characteristic of all type II diabetes. Glucokinase activators will increase the flux of glucose metabolism in β-cells and hepatocytes, which will be coupled to increased insulin secretion. Such agents would be useful for treating type II diabetes.

### SUMMARY OF THE INVENTION

In an embodiment of the present invention, provided are compounds of general formula (I):

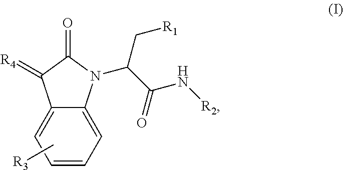

wherein:
R₁ is cycloalkyl or lower alkyl;
R₂ is an unsubstituted or substituted heteroaryl connected by a ring carbon atom to the amine group shown, with one heteroatom being nitrogen which is adjacent to the connecting ring carbon atom, said substituted heteroaryl being substituted at a position other than adjacent to said connecting carbon atom independently with lower alkyl;
R₃ is hydrogen, halogen, lower alkyl, alkoxy, halo-alkoxy, cyano or —SO₂CH₃; and
R₄ is oxygen or absent;
or a pharmaceutically acceptable salt thereof.

In another embodiment of the present invention, provided is a process for the preparation of a compound according to formula I, comprising the steps of: reacting a compound of formula:

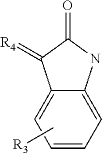

with a compound of formula:

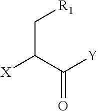

wherein X is halogen and Y is lower alkoxy,
to obtain a compound of formula (I):

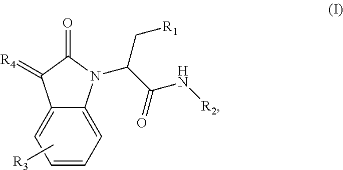

wherein R₁, R₂, R₃ and R₄ are as defined above,
and, optionally, converting the compound according to formula (I) into a pharmaceutically acceptable salt.

In a further embodiment of the invention, provided is a pharmaceutical composition comprising a compound according to formula (I) and a therapeutically inert carrier.

In a still further embodiment of the invention, provided is a method for the treatment or prophylaxis of diabetes, which method comprises the step of administering an effective amount of a compound according to formula (I).

### DETAILED DESCRIPTION OF THE INVENTION

It is to be understood that the terminology employed herein is for the purpose of describing particular embodiments, and is not intended to be limiting. Further, although any methods, devices and materials similar or equivalent to those described herein can be used in the practice or testing of the invention, the preferred methods, devices and materials are now described.

As used herein, the term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

As used herein, the term “acyl” means an optionally substituted alkyl, cycloalkyl, heterocyclic, aryl or heteroaryl group bound via a carbonyl group and includes groups such as acetyl, —C(O)-lower alkyl, branched or unbranched, unsubstituted or substituted with alkoxy or cycloalkyl, —C(O)-cycloalkyl, —C(O)-heterocycloalkyl, unsubstituted or substituted with methyl, —C(O)-aryl, —C(O)-alkoxy, and —C(O)-heteroaryl, unsubstituted or substituted with methyl, and the like.

The term “cycloalkyl” refers to a monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, adamantyl, indanyl and the like. In a preferred embodiment, the “cycloalkyl” moieties can optionally be substituted with one, two, three or four substituents, with the understanding that said substituents are not, in turn, substituted further. Each substituent can independently be, alkyl, alkoxy, halogen, amino, hydroxyl or oxygen (O═) unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl, and the like or those which are specifically exemplified herein.

The term “heterocycloalkyl” denotes a mono- or polycyclic alkyl ring, wherein one, two or three of the carbon ring atoms is replaced by a heteroatom such as N, O or S. Examples of heterocycloalkyl groups include, but are not limited to, morpholinyl, thiomorpholinyl, piperazinyl, piperidinyl, pyrrolidinyl, tetrahydropyranyl, tetrahydrofuranyl, 1,3-dioxanyl and the like. The heterocycloalkyl groups may be unsubstituted or substituted and attachment may be through their carbon frame or through their heteroatom(s) where appropriate, with the understanding that said substituents are not, in turn, substituted further.

The term “lower alkyl”, alone or in combination with other groups, refers to a branched or straight-chain alkyl radical of one to nine carbon atoms, preferably one to six carbon atoms, more preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

The term “aryl” refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthalene, 1,2-dihydronaphthalene, indanyl, 1H-indenyl and the like.

The alkyl, lower alkyl and aryl groups may be substituted or unsubstituted. When substituted, there will generally be, for example, 1 to 4 substituents present, with the understanding that said substituents are not, in turn, substituted further. These substituents may optionally form a ring with the alkyl, loweralkyl or aryl group with which they are connected. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl, more preferably, for example, methoxy and ethoxy), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters(e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono-or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl and carbolinyl).

The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group.

The heteroaryl group described above may be substituted independently with one, two, or three substituents, with the understanding that said substituents are not, in turn, substituted further. These substituents may optionally form a ring with the heteroaryl group to which they are connected. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl, benzothiazoyl and carbolinyl).

As used herein, the term “alkoxy” means alkyl-O—; and “alkoyl” means alkyl-CO—. Alkoxy substituent groups or alkoxy-containing substituent groups may be substituted by, for example, one or more alkyl groups, with the understanding that said substituents are not, in turn, substituted further.

As used herein, the term “halogen” means a fluorine, chlorine, bromine or iodine radical, preferably a fluorine, chlorine or bromine radical, and more preferably a fluorine or chlorine radical.

Compounds of formula I can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). The invention embraces all of these forms.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Particularly preferred are fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminum salts.

In the practice of the method of the present invention, an effective amount of any one of the compounds of this invention or a combination of any of the compounds of this invention or a pharmaceutically acceptable salt thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases. Thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. Preferably, the therapeutically effective amount is in an amount of from about 1 mg to about 500 mg per day.

It will be appreciated, that the compounds of general formula I in this invention may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

The present invention provides novel compounds of general formula (I):

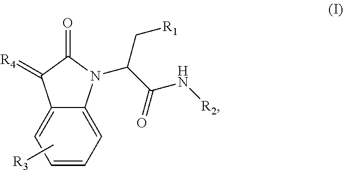

wherein:
R₁ is cycloalkyl or lower alkyl;
R₂ is an unsubstituted or substituted heteroaryl connected by a ring carbon atom to the amine group shown, with one heteroatom being nitrogen which is adjacent to the connecting ring carbon atom, said substituted heteroaryl being substituted at a position other than adjacent to said connecting carbon atom independently with lower alkyl;
R₃ is hydrogen, halogen, lower alkyl, alkoxy, halo-alkoxy, cyano or —SO₂CH₃; and
R₄ is oxygen or absent;
or a pharmaceutically acceptable salt thereof.

In an embodiment of the invention, provided are compounds of formula (I) wherein R₁ is cycloalkyl.

In an embodiment of the invention, provided are compounds of formula (I) wherein R₁ is cyclopentanyl.

In an embodiment of the invention, provided are compounds of formula (I) wherein R₁ is lower alkyl.

In an embodiment of the invention, provided are compounds of formula (I) wherein R₁ is isopropyl.

In an embodiment of the invention, provided are compounds of formula (I) wherein R₂ is unsubstituted heteroaryl.

In an embodiment of the invention, provided are compounds of formula (I) wherein R₂ is unsubstituted pyrazinyl, unsubstituted thiazolyl, unsubstituted pyrazolyl or unsubstituted pyridinyl.

In an embodiment of the invention, provided are compounds of formula (I) wherein R₂ is heteroaryl substituted with lower alkyl.

In an embodiment of the invention, provided are compounds of formula (I) wherein R₂ is substituted pyrazinyl, substituted thiazolyl, substituted pyrazolyl or substituted pyridinyl, wherein said substituent is methyl, 2-hydroxy-2-methyl propyl or 2,3-dihydroxy-propyl.

In an embodiment of the invention, provided are compounds of formula (I) wherein R₃ is hydrogen or halogen.

In an embodiment of the invention, provided are compounds of formula (I) wherein R₃ is lower alkyl, alkoxy or halo-alkoxy.

In an embodiment of the invention, provided are compounds of formula (I) wherein R₃ is hydrogen, bromine, fluorine, chlorine, —OCH₃, —OCF₃, methyl, cyano or —SO₂CH₃.

In an embodiment of the invention, provided are compounds of formula (I) wherein R₃ is hydrogen.

In an embodiment of the invention, provided are compounds of formula (I) wherein R₃ is bromine, fluorine or chlorine.

In an embodiment of the invention, provided are compounds of formula (I) wherein R₃ is methyl.

In an embodiment of the invention, provided are compounds of formula (I) wherein R₃ is —OCH₃ or —OCF₃.

In an embodiment of the invention, provided are compounds of formula (I) wherein R₃ is cyano or —SO₂CH₃.

In an embodiment of the invention, provided are compounds of formula (I) wherein R₄ is oxygen.

In an embodiment of the invention, provided are compounds of formula (I) wherein R₄ is absent.

Particular compounds of formula (I) include the following:
3-Cyclopentyl-2-(2,3-dioxo-2,3-dihydro-indol-1-yl)-N-pyrazin-2-yl-propionamide;
3-Cyclopentyl-2-(2-oxo-2,3-dihydro-indol-1-yl)-N-pyrazin-2-yl-propionamide;
3-Cyclopentyl-2-(2-oxo-2,3-dihydro-indol-1-yl)-N-thiazol-2-yl-propionamide;
3-Cyclopentyl-2-(2,3-dioxo-2,3-dihydro-indol-1-yl)-N-thiazol-2-yl-propionamide;
3-Cyclopentyl-2-(2,3-dioxo-2,3-dihydro-indol-1-yl)-N-(1-methyl-1H-pyrazol-3-yl)-propionamide;
3-Cyclopentyl-2-(2,3-dioxo-2,3-dihydro-indol-1-yl)-N-pyridin-2-yl-propionamide;
3-Cyclopentyl-N-(1-methyl-1H-pyrazol-3-yl)-2-(2-oxo-2,3-dihydro-indol-1-yl)-propionamide;
3-Cyclopentyl-2-(2-oxo-2,3-dihydro-indol-1-yl)-N-pyridin-2-yl-propionamide;
2-(2,3-Dioxo-2,3-dihydro-indol-1-yl)-4-methyl-pentanoic acid pyridin-2-ylamide;
2-(2,3-Dioxo-2,3-dihydro-indol-1-yl)-4-methyl-pentanoic acid thiazol-2-ylamide;
4-Methyl-2-(2-oxo-2,3-dihydro-indol-1-yl)-pentanoic acid thiazol-2-ylamide;
4-Methyl-2-(2-oxo-2,3-dihydro-indol-1-yl)-pentanoic acid pyridin-2-ylamide;
2-(4-Chloro-2,3-dioxo-2,3-dihydro-indol-1-yl)-3-cyclopentyl-N-pyrazin-2-yl-propionamide;
2-(4-Chloro-2,3-dioxo-2,3-dihydro-indol-1-yl)-3-cyclopentyl-N-pyridin-2-yl-propionamide;
2-(4-Chloro-2,3-dioxo-2,3-dihydro-indol-1-yl)-3-cyclopentyl-N-(1-methyl-1H-pyrazol-3-yl)-propionamide;
2-(4-Chloro-2,3-dioxo-2,3-dihydro-indol-1-yl)-3-cyclopentyl-N-thiazol-2-yl-propionamide;
2-(4-Chloro-2,3-dioxo-2,3-dihydro-indol-1-yl)-4-methyl-pentanoic acid pyrazin-2-ylamide;
2-(4-Chloro-2,3-dioxo-2,3-dihydro-indol-1-yl)-4-methyl-pentanoic acid pyridin-2-ylamide;
2-(4-Chloro-2,3-dioxo-2,3-dihydro-indol-1-yl)-4-methyl-pentanoic acid (1-methyl-1H-pyrazol-3-yl)-amide;
2-(4-Chloro-2,3-dioxo-2,3-dihydro-indol-1-yl)-4-methyl-pentanoic acid thiazol-2-ylamide;
2-(4-Chloro-2-oxo-2,3-dihydro-indol-1-yl)-3-cyclopentyl-N-pyridin-2-yl-propionamide;
2-(4-Chloro-2-oxo-2,3-dihydro-indol-1-yl)-3-cyclopentyl-N-thiazol-2-yl-propionamide;
2-(4-Chloro-2-oxo-2,3-dihydro-indol-1-yl)-3-cyc lop entyl-N-(1-methyl-1H-pyrazol-3-yl)-propionamide;
2-(4-Chloro-2-oxo-2,3-dihydro-indol-1-yl)-3-cyclopentyl-N-pyrazin-2-yl-propionamide;
2-(4-Chloro-2-oxo-2,3-dihydro-indol-1-yl)-4-methyl-pentanoic acid pyrazin-2-ylamide;
2-(4-Chloro-2-oxo-2,3-dihydro-indol-1-yl)-4-methyl-pentanoic acid pyridin-2-ylamide;
2-(4-Chloro-2-oxo-2,3-dihydro-indol-1-yl)-4-methyl-pentanoic acid (1-methyl-1H-pyrazol-3-yl)-amide;
2-(4-Chloro-2-oxo-2,3-dihydro-indol-1-yl)-4-methyl-pentanoic acid thiazol-2-ylamide;
3-Cyclopentyl-2-(2,3-dioxo-5-trifluoromethoxy-2,3-dihydro-indol-1-yl)-N-pyrazin-2-yl-propionamide;
3-Cyclopentyl-2-(2,3-dioxo-5-trifluoromethoxy-2,3-dihydro-indol-1-yl)-N-pyridin-2-yl-propionamide;
3-Cyclopentyl-2-(2,3-dioxo-5-trifluoromethoxy-2,3-dihydro-indol-1-yl)-N-(1-methyl-1H-pyrazol-3-yl)-propionamide;
3-Cyclopentyl-2-(2,3-dioxo-5-trifluoromethoxy-2,3-dihydro-indol-1-yl)-N-thiazol-2-yl-propionamide;
3-Cyclopentyl-2-(2-oxo-5-trifluoromethoxy-2,3-dihydro-indol-1-yl)-N-pyridin-2-yl-propionamide;
3-Cyclopentyl-N-(1-methyl-1H-pyrazol-3-yl)-2-(2-oxo-5-trifluoromethoxy-2,3-dihydro-indol-1-yl)-propionamide;
3-Cyclopentyl-2-(2-oxo-5-trifluoromethoxy-2,3-dihydro-indol-1-yl)-N-thiazol-2-yl-propionamide;
3-Cyclopentyl-2-(2-oxo-5-trifluoromethoxy-2,3-dihydro-indol-1-yl)-N-pyrazin-2-yl-propionamide;
2-(2,3-Dioxo-5-trifluoromethoxy-2,3-dihydro-indol-1-yl)-4-methyl-pentanoic acid pyrazin-2-ylamide;
2-(2,3-Dioxo-5-trifluoromethoxy-2,3-dihydro-indol-1-yl)-4-methyl-pentanoic acid pyridin-2-ylamide;
2-(2,3-Dioxo-5-trifluoromethoxy-2,3-dihydro-indol-1-yl)-4-methyl-pentanoic acid (1-methyl-1H-pyrazol-3-yl)-amide;
2-(2,3-Dioxo-5-trifluoromethoxy-2,3-dihydro-indol-1-yl)-4-methyl-pentanoic acid thiazol-2-ylamide;
4-Methyl-2-(2-oxo-5-trifluoromethoxy-2,3-dihydro-indol-1-yl)-pentanoic acid thiazol-2-ylamide;
4-Methyl-2-(2-oxo-5-trifluoromethoxy-2,3-dihydro-indol-1-yl)-pentanoic acid (1-methyl-1H-pyrazol-3-yl)-amide;
4-Methyl-2-(2-oxo-5-trifluoromethoxy-2,3-dihydro-indol-1-yl)-pentanoic acid pyridin-2-ylamide;
4-Methyl-2-(2-oxo-5-trifluoromethoxy-2,3-dihydro-indol-1-yl)-pentanoic acid pyrazin-2-ylamide;
3-Cyclopentyl-2-(5-methoxy-2,3-dioxo-2,3-dihydro-indol-1-yl)-N-pyrazin-2-yl-propionamide;
3-Cyclopentyl-2-(5-methoxy-2,3-dioxo-2,3-dihydro-indol-1-yl)-N-pyridin-2-yl-propionamide;
3-Cyclopentyl-2-(5-methoxy-2,3-dioxo-2,3-dihydro-indol-1-yl)-N-(1-methyl-1H-pyrazol-3-yl)-propionamide;
3-Cyclopentyl-2-(5-methoxy-2,3-dioxo-2,3-dihydro-indol-1-yl)-N-thiazol-2-yl-propionamide;
3-Cyclopentyl-2-(5-methoxy-2-oxo-2,3-dihydro-indol-1-yl)-N-pyridin-2-yl-propionamide;
3-Cyclopentyl-2-(5-methoxy-2-oxo-2,3-dihydro-indol-1-yl)-N-(1-methyl-1H-pyrazol-3-yl)-propionamide;
3-Cyclopentyl-2-(5-methoxy-2-oxo-2,3-dihydro-indol-1-yl)-N-thiazol-2-yl-propionamide;
3-Cyclopentyl-2-(5-methoxy-2-oxo-2,3-dihydro-indol-1-yl)-N-pyrazin-2-yl-propionamide;
2-(5-Methoxy-2,3-dioxo-2,3-dihydro-indol-1-yl)-4-methyl-pentanoic acid pyrazin-2-ylamide;
2-(5-Methoxy-2,3-dioxo-2,3-dihydro-indol-1-yl)-4-methyl-pentanoic acid pyridin-2-ylamide;
2-(5-Methoxy-2,3-dioxo-2,3-dihydro-indol-1-yl)-4-methyl-pentanoic acid thiazol-2-ylamide;
2-(5-Methoxy-2-oxo-2,3-dihydro-indol-1-yl)-4-methyl-pentanoic acid thiazol-2-ylamide;
2-(5-Methoxy-2-oxo-2,3-dihydro-indol-1-yl)-4-methyl-pentanoic acid pyridin-2-ylamide;
2-(5-Methoxy-2-oxo-2,3-dihydro-indol-1-yl)-4-methyl-pentanoic acid pyrazin-2-ylamide;
3-Cyclopentyl-N-[1-(2-hydroxy-2-methyl-propyl)-1H-pyrazol-3-yl]-2-(2-oxo-2,3-dihydro-indol-1-yl)-propionamide;
2-(5-Bromo-2,3-dioxo-2,3-dihydro-indol-1-yl)-3-cyclopentyl-N-pyrazin-2-yl-propionamide;
2-(5-Bromo-2,3-dioxo-2,3-dihydro-indol-1-yl)-3-cyclopentyl-N-pyridin-2-yl-propionamide;
2-(5-Bromo-2,3-dioxo-2,3-dihydro-indol-1-yl)-3-cyclopentyl-N-(1-methyl-1H-pyrazol-3-yl)-propionamide;
2-(5-Bromo-2,3-dioxo-2,3-dihydro-indol-1-yl)-3-cyclopentyl-N-thiazol-2-yl-propionamide;
2-(5-Bromo-2-oxo-2,3-dihydro-indol-1-yl)-3-cyclopentyl-N-pyridin-2-yl-propionamide;
2-(5-Bromo-2-oxo-2,3-dihydro-indol-1-yl)-3-cyclopentyl-N-(1-methyl-1H-pyrazol-3-yl)-propionamide;
2-(5-Bromo-2-oxo-2,3-dihydro-indol-1-yl)-3-cyclopentyl-N-thiazol-2-yl-propionamide;
2-(5-Bromo-2-oxo-2,3-dihydro-indol-1-yl)-3-cyclopentyl-N-pyrazin-2-yl-propionamide;
2-(5-Bromo-2,3-dioxo-2,3-dihydro-indol-1-yl)-4-methyl-pentanoic acid pyrazin-2-ylamide;
2-(5-Bromo-2,3-dioxo-2,3-dihydro-indol-1-yl)-4-methyl-pentanoic acid pyridin-2-ylamide;
2-(5-Bromo-2,3-dioxo-2,3-dihydro-indol-1-yl)-4-methyl-pentanoic acid (1-methyl-1H-pyrazol-3-yl)-amide;
2-(5-Bromo-2,3-dioxo-2,3-dihydro-indol-1-yl)-4-methyl-pentanoic acid thiazol-2-ylamide;
2-(5-Bromo-2-oxo-2,3-dihydro-indol-1-yl)-4-methyl-pentanoic acid thiazol-2-ylamide;
2-(5-Bromo-2-oxo-2,3-dihydro-indol-1-yl)-4-methyl-pentanoic acid (1-methyl-1H-pyrazol-3-yl)-amide;
2-(5-Bromo-2-oxo-2,3-dihydro-indol-1-yl)-4-methyl-pentanoic acid pyridin-2-ylamide;
2-(5-Bromo-2-oxo-2,3-dihydro-indol-1-yl)-4-methyl-pentanoic acid pyrazin-2-ylamide;
3-Cyclopentyl-2-(5-methyl-2,3-dioxo-2,3-dihydro-indol-1-yl)-N-pyrazin-2-yl-propionamide;
3-Cyclopentyl-2-(5-methyl-2,3-dioxo-2,3-dihydro-indol-1-yl)-N-pyridin-2-yl-propionamide;
3-Cyclopentyl-2-(5-methyl-2,3-dioxo-2,3-dihydro-indol-1-yl)-N-(1-methyl-1H-pyrazol-3-yl)-propionamide;
3-Cyclopentyl-2-(5-methyl-2,3-dioxo-2,3-dihydro-indol-1-yl)-N-thiazol-2-yl-propionamide;
3-Cyclopentyl-2-(5-methyl-2-oxo-2,3-dihydro-indol-1-yl)-N-pyridin-2-yl-propionamide;
3-Cyclopentyl-2-(5-methyl-2-oxo-2,3-dihydro-indol-1-yl)-N-(1-methyl-1H-pyrazol-3-yl)-propionamide;
3-Cyclopentyl-2-(5-methyl-2-oxo-2,3-dihydro-indol-1-yl)-N-thiazol-2-yl-propionamide;
3-Cyclopentyl-2-(5-methyl-2-oxo-2,3-dihydro-indol-1-yl)-N-pyrazin-2-yl-propionamide;
4-Methyl-2-(5-methyl-2,3-dioxo-2,3-dihydro-indol-1-yl)-pentanoic acid pyrazin-2-ylamide;
4-Methyl-2-(5-methyl-2,3-dioxo-2,3-dihydro-indol-1-yl)-pentanoic acid pyridin-2-ylamide;
4-Methyl-2-(5-methyl-2,3-dioxo-2,3-dihydro-indol-1-yl)-pentanoic acid (1-methyl-1H-pyrazol-3-yl)-amide;
4-Methyl-2-(5-methyl-2,3-dioxo-2,3-dihydro-indol-1-yl)-pentanoic acid thiazol-2-ylamide;
4-Methyl-2-(5-methyl-2-oxo-2,3-dihydro-indol-1-yl)-pentanoic acid thiazol-2-ylamide;
4-Methyl-2-(5-methyl-2-oxo-2,3-dihydro-indol-1-yl)-pentanoic acid (1-methyl-1H-pyrazol-3-yl)-amide;
4-Methyl-2-(5-methyl-2-oxo-2,3-dihydro-indol-1-yl)-pentanoic acid pyridin-2-ylamide;
4-Methyl-2-(5-methyl-2-oxo-2,3-dihydro-indol-1-yl)-pentanoic acid pyrazin-2-ylamide;
2-(5-Chloro-2,3-dioxo-2,3-dihydro-indol-1-yl)-3-cyclopentyl-N-thiazol-2-yl-propionamide;
2-(5-Chloro-2,3-dioxo-2,3-dihydro-indol-1-yl)-3-cyclopentyl-N-[1-((R)-2,3-dihydroxy-propyl)-1H-pyrazol-3-yl]-propionamide;
2-(5-Chloro-2,3-dioxo-2,3-dihydro-indol-1-yl)-3-cyclopentyl-N-pyridin-2-yl-propionamide;
2-(5-Chloro-2,3-dioxo-2,3-dihydro-indol-1-yl)-3-cyclopentyl-N-(1-methyl-1H-pyrazol-3-yl)-propionamide;
2-(5-Chloro-2,3-dioxo-2,3-dihydro-indol-1-yl)-3-cyclopentyl-N-pyrazin-2-yl-propionamide;
2-(5-Chloro-2-oxo-2,3-dihydro-indol-1-yl)-3-cyclopentyl-N-pyrazin-2-yl-propionamide;
2-(5-Chloro-2-oxo-2,3-dihydro-indol-1-yl)-3-cyclopentyl-N-thiazol-2-yl-propionamide;
2-(5-Chloro-2-oxo-2,3-dihydro-indol-1-yl)-3-cyclopentyl-N-(1-methyl-1H-pyrazol-3-yl)-propionamide;
2-(5-Chloro-2-oxo-2,3-dihydro-indol-1-yl)-3-cyclopentyl-N-pyridin-2-yl-propionamide;
2-(5-Chloro-2,3-dioxo-2,3-dihydro-indol-1-yl)-4-methyl-pentanoic acid thiazol-2-ylamide;
2-(5-Chloro-2,3-dioxo-2,3-dihydro-indol-1-yl)-4-methyl-pentanoic acid pyridin-2-ylamide;
2-(5-Chloro-2,3-dioxo-2,3-dihydro-indol-1-yl)-4-methyl-pentanoic acid pyrazin-2-ylamide;
2-(5-Chloro-2,3-dioxo-2,3-dihydro-indol-1-yl)-4-methyl-pentanoic acid (1-methyl-1H-pyrazol-3-yl)-amide;
2-(5-Chloro-2-oxo-2,3-dihydro-indol-1-yl)-4-methyl-pentanoic acid thiazol-2-ylamide;
2-(5-Chloro-2-oxo-2,3-dihydro-indol-1-yl)-4-methyl-pentanoic acid pyrazin-2-ylamide;
2-(5-Chloro-2-oxo-2,3-dihydro-indol-1-yl)-4-methyl-pentanoic acid (1-methyl-1H-pyrazol-3-yl)-amide;
2-(5-Chloro-2-oxo-2,3-dihydro-indol-1-yl)-4-methyl-pentanoic acid pyridin-2-ylamide;
2-(6-Chloro-2,3-dioxo-2,3-dihydro-indol-1-yl)-4-methyl-pentanoic acid thiazol-2-ylamide;
2-(6-Chloro-2,3-dioxo-2,3-dihydro-indol-1-yl)-4-methyl-pentanoic acid pyridin-2-ylamide;
2-(6-Chloro-2,3-dioxo-2,3-dihydro-indol-1-yl)-4-methyl-pentanoic acid (1-methyl-1H-pyrazol-3-yl)-amide;
2-(6-Chloro-2,3-dioxo-2,3-dihydro-indol-1-yl)-4-methyl-pentanoic acid pyrazin-2-ylamide;
2-(6-Chloro-2-oxo-2,3-dihydro-indol-1-yl)-4-methyl-pentanoic acid pyrazin-2-ylamide;
2-(6-Chloro-2-oxo-2,3-dihydro-indol-1-yl)-4-methyl-pentanoic acid pyridin-2-ylamide;
2-(6-Chloro-2-oxo-2,3-dihydro-indol-1-yl)-4-methyl-pentanoic acid thiazol-2-ylamide;
2-(6-Chloro-2-oxo-2,3-dihydro-indol-1-yl)-4-methyl-pentanoic acid (1-methyl-1H-pyrazol-3-yl)-amide;
2-(6-Chloro-2,3-dioxo-2,3-dihydro-indol-1-yl)-3-cyclopentyl-N-thiazol-2-yl-propionamide;
2-(6-chloro-2,3-dioxo-2,3-dihydro-indol-1-yl)-3-cyclopentyl-N-pyridin-2-yl-propionamide;
2-(6-Chloro-2,3-dioxo-2,3-dihydro-indol-1-yl)-3-cyclopentyl-N-(1-methyl-1H-pyrazol-3-yl)-propionamide;
2-(6-Chloro-2,3-dioxo-2,3-dihydro-indol-1-yl)-3-cyclopentyl-N-pyrazin-2-yl-propionamide;
2-(6-Chloro-2-oxo-2,3-dihydro-indol-1-yl)-3-cyclopentyl-N-pyrazin-2-yl-propionamide;
2-(6-Chloro-2-oxo-2,3-dihydro-indol-1-yl)-3-cyclopentyl-N-pyridin-2-yl-propionamide;
2-(6-Chloro-2-oxo-2,3-dihydro-indol-1-yl)-3-cyclopentyl-N-thiazol-2-yl-propionamide;
2-(6-Chloro-2-oxo-2,3-dihydro-indol-1-yl)-3-cyc lop entyl-N-(1-methyl-1H-pyrazol-3-yl)-propionamide;
3-Cyclopentyl-2-(6-fluoro-2-oxo-2,3-dihydro-indol-1-yl)-N-thiazol-2-yl-propionamide;
3-Cyclopentyl-2-(6-fluoro-2-oxo-2,3-dihydro-indol-1-yl)-N-(1-methyl-1H-pyrazol-3-yl)-propionamide;
3-Cyclopentyl-2-(6-fluoro-2-oxo-2,3-dihydro-indol-1-yl)-N-pyridin-2-yl-propionamide;
3-Cyclopentyl-2-(6-fluoro-2-oxo-2,3-dihydro-indol-1-yl)-N-pyrazin-2-yl-propionamide;
3-Cyclopentyl-2-(6-methyl-2-oxo-2,3-dihydro-indol-1-yl)-N-thiazol-2-yl-propionamide;
3-Cyclopentyl-2-(6-methyl-2-oxo-2,3-dihydro-indol-1-yl)-N-pyrazin-2-yl-propionamide;
3-Cyclopentyl-2-(6-methyl-2-oxo-2,3-dihydro-indol-1-yl)-N-(1-methyl-1H-pyrazol-3-yl)-propionamide;
3-Cyclopentyl-2-(6-methyl-2-oxo-2,3-dihydro-indol-1-yl)-N-pyridin-2-yl-propionamide;
2-(6-Bromo-2-oxo-2,3-dihydro-indol-1-yl)-3-cyclopentyl-N-pyrazin-2-yl-propionamide;
2-(6-Bromo-2-oxo-2,3-dihydro-indol-1-yl)-3-cyclopentyl-N-thiazol-2-yl-propionamide;
2-(6-Bromo-2-oxo-2,3-dihydro-indol-1-yl)-3-cyclopentyl-N-pyridin-2-yl-propionamide;
2-(6-Bromo-2-oxo-2,3-dihydro-indol-1-yl)-3-cyclopentyl-N-(1-methyl-1H-pyrazol-3-yl)-propionamide;
2-(6-Cyano-2-oxo-2,3-dihydro-indol-1-yl)-3-cyclopentyl-N-pyridin-2-yl-propionamide; And
3-Cyclopentyl-2-(6-methanesulfonyl-2-oxo-2,3-dihydro-indol-1-yl)-N-thiazol-2-yl-propionamide.

In another embodiment of the invention, provided is a a process for the preparation of a compound according to formula I, comprising the steps of: reacting a compound of formula:

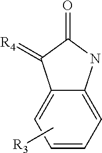

with a compound of formula:

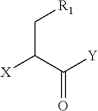

wherein X is halogen and Y is lower alkoxy,
to obtain a compound of formula (I):

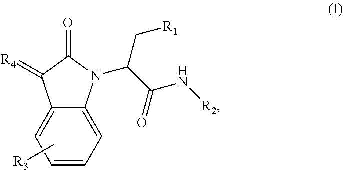

wherein R₁, R₂, R₃ and R₄ are as defined above,
and, optionally, converting the compound according to formula (I) into a pharmaceutically acceptable salt.

In an embodiment of the invention, provided are compounds of formula (I) for use as a therapeutically active substance.

In a further embodiment of the invention, provided is a pharmaceutical composition comprising a compound according to formula (I) and a therapeutically inert carrier.

In another embodiment of the invention, provided is the use of a compound according to formula (I) for the treatment or prophylaxis of diabetes.

In another embodiment of the invention, provided is the use of a compound according to formula (I) for the preparation of a medicament for the treatment or prophylaxis of diabetes.

In another embodiment of the invention, provided is a a compound according formula (I) for the treatment or prophylxis of diabetes.

In another embodiment of the invention, provided is a a compound according formula (I) when manufactured according to a process as disclosed above.

In a still further embodiment of the invention, provided is a method for the treatment or prophylaxis of diabetes, which method comprises the step of administering an effective amount of a compound according to formula (I).

In another embodiment of the invention, provided is an invention as hereinbefore described.

Compounds of the present invention can be prepared beginning with commercially available starting materials, or utilizing general synthetic techniques and procedures known to those skilled in the art. Chemicals may be purchased from companies such as for example Aldrich, Argonaut Technologies, VWR, Lancaster, Princeton, Alfa, Oakwood, TCI, Fluorochem, Apollo, Matrix, Maybridge or Meinoah. Chromatography supplies and equipment may be purchased from such companies as for example Anal.ogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, NJ; Varian Inc., Palo Alto, Calif., and Multigram II Mettler Toledo Instrument Newark, Del. Biotage, ISCO and Analogix columns are pre-packed silica gel columns used in standard chromatography. Final compounds and intermediates were named using the AutoNom2000 feature in the MDL ISIS Draw application.

The compounds of formula I, for example Ia and Ib, can be prepared starting from compounds of formula II and formula IV by the following general reaction scheme.

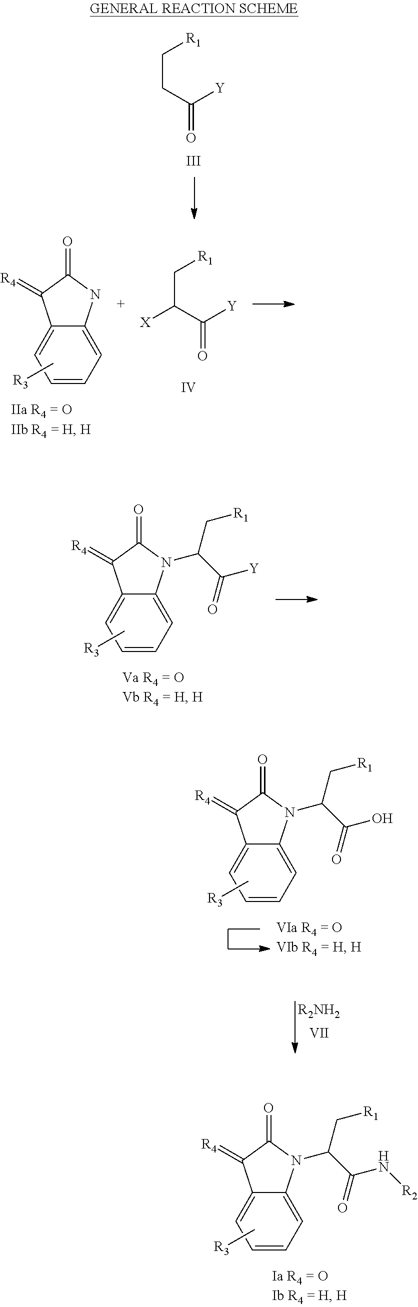

Compounds of formula IIa, where R₄ is oxygen and where R₃ is hydrogen, halogen, lower alkyl, lower alkoxy, perfluoroalkyl, perfluoroalkoxy, aryloxy, nitro, carboalkoxy, thioloweralkyl, alkylsulfonyl, nitrile, aryl or heteroaryl are commercially available or can be prepared using literature procedures (see for example, 6-methoxy-1H-indole-2,3-dione Synthesis, 2009, 21, 3642-3648; 5-furan-2-yl-1H-indole-2,3-dione Tetrahedron, 2005, 61(25), 6082-6087; 4-ethyl-1H-indole-2,3-dione, 4-phenyl-1H-indole-2,3-dione Bioorg. Med. Chem. Lett. 2009, 19(10), 2891-2895). If it is desired to produce 6-methylsulfanyl-1H-indole-2,3-dione, it can be prepared from 6-bromo-1H-indole-2,3-dione utilizing any conventional method of displacing a bromine with a thiomethyl group (see for example J. Org. Chem. 1962, 27, 4708-9; Bull. Chem. Soc. Jpn. 1975, 48, 363-4, Bull. Chem. Soc. Jpn. 1978, 51, 3093-4)

Compounds of formula IIb, where R₄ is absent so as to make it an oxindole and R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl are commercially available or can be prepared using literature procedures (see for example, 6-trifluoromethyl-1,3-dihydro-indol-2-one PCT Int. App. 2007106564; 5-trifluoromethyl-1,3-dihydro-indol-2-one J. Comb. Chem. 2007, 9(4), 566-568; 6-methoxy-1,3-dihydro-indol-2-one Synthesis 1993, (1), 51-3.)

Compounds of formula IIa or formula IIb that are di- or trisubstituted on the phenyl ring with a halogen, lower alkyl, lower alkoxy, perfluoroalkyl, perfluoroalkoxy, aryloxy, nitro, carboalkoxy, thioloweralkyl, alkylsulfonyl, nitrile, aryl or heteroaryl are commercially available or can be prepared using literature procedures (see for example 4,6-dichloro-1H-indole-2,3-dione PCT Int. App. 2008009415; 5,6-dibromo-1H-indole-2,3-dione Bioorg. Med. Chem. 2007, 15(2), 931-938; 5,6-dimethoxy-1,3-dihydro-indol-2-one PCT Int. App. 2005068424; 5,6-dichloro-1,3-dihydro-indol-2-one J. Med. Chem. 2000, 43(12), 2449-2456;). Compounds of formula IIa or formula IIb with di- or trisubstitution on the phenyl ring may be carried through the same steps as outlined below to yield compounds of formula Ia or Ib.

Compounds of formula III, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl and Y is hydroxyl or chloro are commercially available or can be prepared using literature procedures. For example, a compound of formula III, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl and Y is hydroxyl can be prepared by treating an appropriately substituted R₁ derivative with a malonate derivative under standard conditions to produce a substituted malonate, hydrolysis of the malonate to form the diacid and subsequent decarboxylation (see for example, J. Med. Chem. 1990, 33, 263-273). Compounds of formula III, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl and Y is chloro can be prepared from compounds of formula III, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl and Y is hydroxyl using standard conditions to convert an acid to an acid chloride, for example thionyl chloride or oxalyl chloride (see for example Eur. J. Med. Chem. 2009, 44(11), 4616-4621; U.S. Pat. App. Pub. 2010093814).

Compounds of formula IV, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl and Y is a lower alkoxy group, for example methoxy or ethoxy and X is a halogen, for example a bromine can be prepared from compounds of formula III where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl and Y is a hydroxyl by treatment with thionyl chloride, a halogen source, for example bromine or N-bromosuccinimide, and an alcohol, for example methanol or ethanol (see for example J. Am. Chem. Soc. 1948, 70, 3626-7; J. Med. Chem. 1981, 24, 481-90; J. Chem. Soc., Perkin Trans. 2 1985, 2, 209-212).

Compounds of formula IV, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl and Y is lower alkoxy, for example, methoxy or ethoxy and X is a halogen, for example bromine can be prepared from compounds of formula III where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl and Y is a chlorine by treatment with a halogen source, for example bromine or N-bromosuccinimide, and an alcohol, for example methanol or ethanol (see for example Tet. Lett. 1994, 35, 4915; PCT Int. Appl. WO2008079028).

Compounds of formula IV, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, X is a halogen, for example, bromine and Y is lower alkoxy, for example, methoxy or ethoxy can be prepared from compounds of formula IV where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, X is an amino or a protected amino group, and Y is hydroxyl or a lower alkoxy, for example, methoxy or ethoxy by any conventional methods used to convert an amino functionality to a halogen, for example a bromine. An example of a method to convert an amino group to a halogen, for example a bromine, utilizes the formation of a diazonium species which can then be converted in situ to a halogen, for example a bromine (see for example Tet. Asym. 1993, 4(6), 1141-1152; J. Org. Chem. 1993, 58, 1159-166; Synthesis, 1999, 4, 583-585). The resulting halo acid, compound of formula IV, where X is a halogen, for example a bromine, and Y is a hydroxyl, may be converted to the appropriately functionalized halo esters where X is a halogen, for example a bromine, and Y is lower alkoxy, for example, a methoxy or ethoxy using any conventional method of converting an acid to an ester.

A number of amino acids, compounds of formula IV where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, X is an amino or a protected amino group, and Y is hydroxy or a lower alkoxy, are available from commercial sources. When the amino acids, compounds of formula IV where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, X is an amino or a protected amino group, and Y is hydroxy or lower alkoxy, for example methoxy or ethoxy, are not commercially available they can be prepared using literature methods (see for example U.S. application Ser. No. 12/400,319).

Compounds of formula IV, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, X is an oxygen based leaving group, for example tosylate, triflate or mesylate and Y is lower alkoxy, for example a methoxy or ethoxy, can be prepared from compounds of formula IV where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, X is a hydroxyl group, and Y is lower alkoxy, for example, a methoxy or ethoxy, by any conventional methods used to convert a hydroxyl group to a tosylate, mesylate or triflate (see for example PCT Int. App. 2008065409; J. Org. Chem. 2004, 69(11) 3964-3967; Synlett 2010, 3, 470-474; Org. Lett. 2009, 11(4), 807-810; PCTInt. App. 2009080722, Angew. Chem., Int. Ed. Engl. 2008, 47(29), 5451-5455).

Compounds of formula IV, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, X is a hydroxyl and Y is lower alkoxy, for example a methoxy or ethoxy, may be commercially available or can be prepared by literature procedures. Compounds of formula IV, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, X is a hydroxyl and Y is lower alkoxy, for example a methoxy or ethoxy, can be prepared from compounds of formula III where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl and Y is lower alkoxy, for example a methoxy or ethoxy, by any method of oxidizing an enolate with reagents such as the Vedejs reagent or phenylsulfonyl phenyl oxaziridine (see for example Tetrahedron: Asymmetry 2009, 20(6-8), 921-944; PCTInt. App.2008053331; Chen, B.-C., Zhou, P., Davis, F. A. and Ciganek, E. Organic Reactions; John Wiley & Sons, Inc.: New York, 2003, 1-356., Helv. Chim. Acta 1986, 69(3), 615-20; Tetrahedron Lett. 1985, 26(2), 203-6).

Compounds of formula Va, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl, R₄ is oxygen and Y is lower akoxy, for example, a methoxy or ethoxy, can be prepared by alkylating compounds of formula IIa, where, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl and R₄ is oxygen using standard alkyating conditions, such as treatment with sodium hydride, calcium hydride, potassium carbonate, or potassium t-butoxide (see for example PCT Int. App. 2008046083; Bioorg. Med. Chem. 2008, 16(8), 4222-4232; PCT Int. App. 2007091147; Bioorg. Med. Chem. Lett. 2007, 17(20), 5630-5633; PCT Int. App. 2006106426; Tetrahedron: Asymmetry 2009, 20(13), 1500-1505; Chem. Heterocycl. Compd. (N.Y.) 2008, 44(9), 1123-1128; J. Med. Chem. 2008, 51(24), 8057-8067; J. Med. Chem. 2006, 49(23), 6704-6715; Bioorg. Med. Chem. 2005, 13(9), 3249-3261; Can. J. Chem. 2009, 87(4), 591-599; J. Med. Chem. 2009, 52(11), 3484-3495) followed by treatment with a compound of formula IV, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, X is a halogen, for example a bromine, or an oxygen containing leaving group, for example a tosylate, mesylate or triflate.

Compounds of formula Vb, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl, R₄ is oxygen and Y is lower alkoxy, for example a methoxy or ethoxy, can be prepared by alkylating compounds of formula IIb, where, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl and R₄ is absent so as to make it an oxindole using standard alkyating conditions, such as treatment with sodium hydride or potassium carbonate (see for example Macromolecules 2009, 42(22), 8718-8724; J. Med. Chem. 2009, 52(8), 2384-2392; PCT Int. App. 2008138939; U.S. Pat. App. Pub.2006211603; Bioorg. Med. Chem. Lett. 2002, 12(21), 3105-3109; PCT Int. App. 2005016883) followed by treatment with a compound of formula IV, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, X is a halogen, for example bromine, or an oxygen containing leaving group, for example a tosylate, mesylate or triflate.

Compounds of formula VIa, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl and R₄ is oxygen can be prepared by hydrolysis of compounds of formula Va, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl, R₄ is oxygen and Y is lower alkoxy or benzyloxy, for example methoxy, ethoxy, benzyloxy or t-butoxy, under standard conditions (see for example, Greene, T. W. Protective Groups in Organic Synthesis; John Wiley & Sons, Inc.: New York, 1991).

Compounds of formula VIb, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl and R₄ is absent so as to make it an oxindole can be prepared by hydrolysis of compounds of formula Vb, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl, R₄ is absent so as to make it an oxindole, and Y is lower alkoxy or benzyloxy, for example methoxy, ethoxy, benzyloxy or t-butoxy, under standard conditions (see for example, Greene, T. W. Protective Groups in Organic Synthesis; John Wiley & Sons, Inc.: New York, 1991).

Alternatively, compounds of formula VIb, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl and R₄ is absent so as to make it an oxindole can be prepared by reducing compounds of formula VIa, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, R₃ is hydrogen, halogen, lower alkyl, lower alkoxy, aryloxy, perfluoroalkoxy, perfluoroalkyl, nitro, carboalkoxy, thioether or thioloweralkyl, alkylsulfonyl, nitrile, aryl or heteroaryl and R₄ is oxygen with reagents such as hydrazine hydrate (see for example Bioorg. Med. Chem. Lett. 2010, 20(8), 2658-2664; Bioorg. Med. Chem. 2010, 18(9), 3004-3011; Bioorg. Med. Chem. 2010, 18(4), 1482-1496; J. Am. Chem. Soc.2004, 126(20), 6347-6355; J. Med. Chem. 1986, 29(6), 939-47; U.S. Pat. App. Pub. 2006079520).

Compounds of formula VII, where R₂ is an unsubstituted or substituted heteroaryl or heterocycloalkyl group may be commercially available or prepared by literature procedures (see for example, U.S. Pat. No. 7,741,327)

Compounds of formula Ia, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl, R₄ is oxygen and R₂ is an unsubstituted or substituted heteroaryl or heterocycloalkyl group can be prepared by treating compounds of formula VIa, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl, R₄ is oxygen with compounds of formula VII where R₂ is an unsubstituted or substituted heteroaryl or heterocycloalkyl group, for example unsubstituted or substituted 1H-pyrazol-3-yl, pyrazin-2-yl, pyridin-2-yl, thiazol-2-yl, under any conventional method to form an amide bond, for example forming an acid chloride and adding the amine or using the acid and amine with such coupling reagents as (benzotriazol-1-yloxytris(dimethylamino)phosphonium hexafluorophosphate (see for example Tetrahedron, 2005, 61, 10827; U.S. Pat. No. 7,741,327). If the compounds of formula Ia are a mixture of enantiomers or diastereomers, the appropriate chromatographic techniques, such as supercritical fluid chromatography, may be utilized to produce chirally pure or chirally enriched compounds of formula Ia.

Compounds of formula Ib, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl, R₄ is absent so as to make it an oxindole and R₂ is an unsubstituted or substituted heteroaryl or heterocycloalkyl group, for example unsubstituted or substituted 1H-pyrazol-3-yl, pyrazin-2-yl, pyridin-2-yl, thiazol-2-yl, can be prepared by treating compounds of formula VIb, where R₁ is aryl, substituted aryl, heteroaryl, substituted heteroaryl, alkyl, cycloalkyl, substituted cycloalkyl, heteroalkyl, substituted heteroalkyl, heterocycloalkyl, or substituted heterocycloalkyl, R₃ is hydrogen, halogen, lower alkyl, lower alkoxy, aryloxy, perfluoroalkoxy, perfluoroalkyl, nitro, carboalkoxy, thioloweralkyl, alkylsulfonyl, nitrile, aryl or heteroaryl, R₄ is hydrogen with compounds of formula VII where R₂ is an unsubstituted or substituted heteroaryl or heterocycloalkyl group under any conventional method to form an amide bond, for example forming an acid chloride and adding the amine or using the acid and amine with such coupling reagents as (benzotriazol-1-yloxytris(dimethylamino)phosphonium hexafluorophosphate (see for example Tetrahedron, 2005, 61, 10827; U.S. Pat. No. 7,741,327). If the compounds of formula Ib are a mixture of enantiomers or diastereomers, the appropriate chromatographic techniques, such as supercritical fluid chromatography, may be utilized to produce chirally pure or chirally enriched compounds of formula Ib.

The compounds of formula Ia or formula Ib having functional groups that may need transformation, conversion or protection may be transformed, converted or deprotected to the desired functionality using conventional methods at an appropriate intermediate step or after the amide coupling step of the synthesis (under similar conditions to those described in Greene, T. W. Protective Groups in Organic Synthesis; John Wiley & Sons, Inc.: New York, 1991). Such conversions may include saponification of an ester to an acid or alcohol under basic conditions, removal of acetals or ketals used to afford aldehydes, ketones or diols, removal of a silyl protecting group from an alcohol, conversion of an acid to an amide, conversion of an olefin to an alcohol, diol, aldehyde, acid or ester or removal of a protecting group from an amine nitrogen. Such conversion of functional groups can include the oxidation of a thiomethyl group to a sulfone using oxone (see for example J. Org. Chem. 2010, 75(13), 4652-4655).

Compounds of formula Ia and formula Ib can be obtained as chirally pure or chirally enriched enantiomers or diastereomers from the corresponding enantiomeric or diastereomeric mixtures of compounds of formula Ia and formula Ib using standard chromatographic techniques, such as supercritical fluid chromatography or high pressure liquid chromatography on chiral supports. Accordingly, separation into chirally pure or chirally enriched enantiomers or diastereomers may be achieved along any step of the reaction sequence by a variety or methods, such as chromatographic techniques, resolution steps, using a chiral salt or any other means known by those skilled in the art to achieve chiral enrichment or separation.

The invention will now be further described in the Examples below, which are intended as an illustration only and do not limit the scope of the invention.

### EXAMPLES

### Example 1

### 3-Cyclopentyl-2-(2,3-dioxo-2,3-dihydro-indol-1-yl)-N-pyrazin-2-yl-propionamide

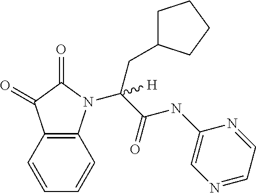

A solution of 3-cyclopentyl-propionic acid (28.44 g) in carbon tetrachloride (20 mL) was treated with thionyl chloride (58.0 mL) and heated at 65° C. for 30 min. The reaction mixture was then removed from the oil bath and a mixture of N-bromosuccinimide (42.8 g) and a 48% aqueous hydrobromic acid solution (20 drops) in carbon tetrachloride (100 mL) was added dropwise. The resulting mixture was then heated at 70° C. for 10 min and then 85° C. for 3 h. After this time, the reaction was cooled to 0° C. in an ice bath and then treated dropwise with methanol (40-50 mL). The mixture was then filtered through a celite plug to remove the insoluble material and the filterate concentrated in vacuo. The residue was then dissolved in pentane (400 mL) and washed with water (400 mL) and the aqueous layer was then extracted with pentane (400 mL). The organic layers were then combined and dried over sodium sulfate, filtered and the filterate concentrated in vacuo. The crude material was purified using flash column chromatography (silica gel, 5% ethyl acetate/hexanes) to afford 2-bromo-3-cyclopentyl-propionic acid methyl ester (36.25 g, 77%) as a yellow oil.

A stirred suspension of sodium hydride (196 mg) in N,N-dimethylformamide (15 mL) at 0° C. was treated with a solution of 1H-indole-2,3-dione (1.0 g) in N,N-dimethylformamide. The reaction mixture was stirred for 30 min at 0° C. and then 2-bromo-3-cyclopentyl-propionic acid methyl ester (1.92 g) was added and it was stirred for 1 h at 0° C. It was then slowly allowed to warm to room temperature and stirred for another 3 h at room temperature. After this time, the reaction mixture was diluted with water and extracted with methylene chloride. The organic layers were combined and dried over sodium sulfate, filtered and the filterate concentrated in vacuo. The crude material was purified by column chromatography (silica gel) to afford 3-cyclopentyl-2-(2,3-dioxo-2,3-dihydro-indol-1-yl)-propionic acid methyl ester (1.0 g, 49%).

A mixture of 3-cyclopentyl-2-(2,3-dioxo-2,3-dihydro-indol-1-yl)-propionic acid methyl ester (1.0 g) and tetrahydrofuran:water (1:1, 20 mL) was stirred at room temperature and treated with lithium hydroxide monohydrate (279 mg). The reaction mixture was then stirred for 2 h at room temperature. After this time, the reaction mixture was concentrated in vacuo to remove the tetrahydrofuran. The remaining aqueous layer was diluted with water and then acidified to pH=2 with a 1N aqueous hydrochloric acid solution. The aqueous layer was then extracted with ethyl acetate and the organic layers combined, dried over sodium sulfate, filtered and the filterate concentrated in vacuo to afford 3-cyclopentyl-2-(2,3-dioxo-2,3-dihydro-indol-1-yl)-propionic acid (900 mg, 94).

A mixture of 3-cyclopentyl-2-(2,3-dioxo-2,3-dihydro-indol-1-yl)-propionic acid (100 mg) in methylene chloride (5 mL) and N,N-dimethylformamide (one drop) at room temperature was treated with oxalyl chloride (53 μL) and stirred for 30 min at room temperature. After this time the reaction mixture was concentrated in vacuo. The residue was dissolved in tetrahydrofuran (10 mL) and then treated with pyrazin-2-ylamine (40 mg) and 2,6-lutidine (100 μL). The resulting reaction mixture was then stirred overnight at room temperature. After this time, the reaction mixture was diluted with ethyl acetate, and washed with a 1N aqueous hydrochloric acid solution, saturated aqueous sodium bicarbonate solution and brine. The organic layers were dried over sodium sulfate, filtered and the filterate concentrated in vacuo. The crude material was purified by column chromatography (silica gel) to afford 3-cyclopentyl-2-(2,3-dioxo-2,3-dihydro-indol-1-yl)-N-pyrazin-2-yl-propionamide (20 mg, 16%): LCMS ESI calcd for C₂₀H₂₀N₄O₃ (M+H)⁺ 365, observed 365.

### Example 2

### 3-Cyclopentyl-2-(2-oxo-2,3-dihydro-indol-1-yl)-N-pyrazin-2-yl-propionamide

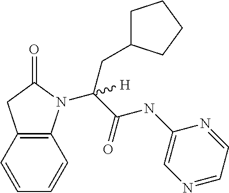

A mixture of 3-cyclopentyl-2-(2,3-dioxo-2,3-dihydro-indol-1-yl)-propionic acid (prepared as in Example 1, 450 mg) and hydrazine hydrate (2 mL) were placed in a sealed tube and heated at 120° C. for 25 min. The reaction mixture was then cooled to room temperature and concentrated in vacuo. The residue was dissolved in methylene chloride and washed with a 0.5N aqueous hydrochloric acid solution. The organic layer was then dried over sodium sulfate, filtered and the filterate concentrated in vacuo to afford 3-cyclopentyl-2-(2-oxo-2,3-dihydro-indol-1-yl)-propionic acid (370 mg, 86%).

A mixture of 3-cyclopentyl-2-(2-oxo-2,3-dihydro-indol-1-yl)-propionic acid (75 mg) and benzene (3 mL) were treated with thionyl chloride (64 μL) and heated at 60° C. for 1 h. After this time, the mixture was concentrated in vacuo to remove the benzene and the residue was dissolved in tetrahydrofuran (3 mL). The resulting solution was then treated with pyrazin-2-ylamine (31 mg) and 2,6-lutidine (81 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 3-cyclopentyl-2-(2-oxo-2,3-dihydro-indol-1-yl)-N-pyrazin-2-yl-propionamide (20 mg, 21%) as an orange solid: LCMS ESI calcd for C₂₀H₂₂N₄O₂ (M+H)⁺ 351, observed 351.

### Example 3

### 3-Cyclopentyl-2-(2-oxo-2,3-dihydro-indol-1-yl)-N-thiazol-2-yl-propionamide

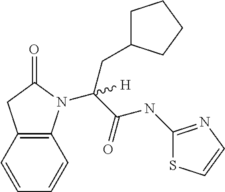

A mixture of 3-cyclopentyl-2-(2-oxo-2,3-dihydro-indol-1-yl)-propionic acid (prepared as in Example 2, 75 mg) and benzene (3 mL) were treated with thionyl chloride (64 μL) and heated at 60° C. for 1 h. After this time, the mixture was concentrated in vacuo to remove the benzene and the residue was dissolved in tetrahydrofuran (3 mL). The resulting solution was then treated with thiazol-2-ylamine (33 mg) and 2,6-lutidine (81 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 3-cyclopentyl-2-(2-oxo-2,3-dihydro-indol-1-yl)-N-thiazol-2-yl-propionamide (30 mg, 31%) as an orange solid: LCMS ESI calcd for C₁₉H₂₁N₃O₂S (M+H)⁺ 356, observed 356.

### Example 4

### 3-Cyclopentyl-2-(2,3-dioxo-2,3-dihydro-indol-1-yl)-N-thiazol-2-yl-propionamide

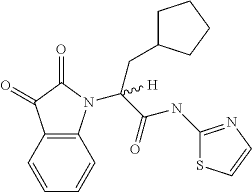

A mixture of 3-cyclopentyl-2-(2,3-dioxo-2,3-dihydro-indol-1-yl)-propionic acid (prepared as in Example 1, 100 mg) and thiazol-2-ylamine (42 mg) in N,N-dimethylformamide (2 mL) at room temperature was treated with N,N-diisopropylethyl amine (100 μL) and (benzotriazol-1-yloxytris(dimethylamino)phosphonium hexafluorophosphate (231 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 3-cyclopentyl-2-(2,3-dioxo-2,3-dihydro-indol-1-yl)-N-thiazol-2-yl-propionamide (35 mg, 27%) as an orange solid: LCMS ESI calcd for C₁₉H₁₉N₃O₃S (M+H)⁺ 370, observed 370.

### Example 5

### 3-Cyclopentyl-2-(2,3-dioxo-2,3-dihydro-indol-1-yl)-N-(1-methyl-1H-pyrazol-3-yl)-propionamide

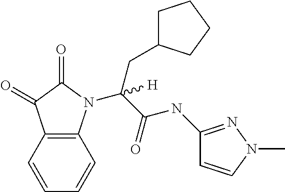

A mixture of 3-cyclopentyl-2-(2,3-dioxo-2,3-dihydro-indol-1-yl)-propionic acid (prepared as in Example 1, 100 mg) and 1-methyl-1H-pyrazol-3-ylamine (42 mg) in N,N-dimethylformamide (2 mL) at room temperature was treated with N,N-diisopropylethyl amine (100 μL) and (benzotriazol-1-yloxytris(dimethylamino)phosphonium hexafluorophosphate (231 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 3-cyclopentyl-2-(2,3-dioxo-2,3-dihydro-indol-1-yl)-N-(1-methyl-1H-pyrazol-3-yl)-propionamide (40 mg, 31%) as an orange solid: LCMS ESI calcd for C₂₀H₂₂N₄O₃ (M+H)⁺ 367, observed 367.

### Example 6

### 3-Cyclopentyl-2-(2,3-dioxo-2,3-dihydro-indol-1-yl)-N-pyridin-2-yl-propionamide

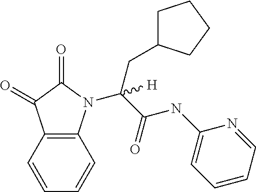

A mixture of 3-cyclopentyl-2-(2,3-dioxo-2,3-dihydro-indol-1-yl)-propionic acid (prepared as in Example 1, 100 mg) and pyridin-2-ylamine (42 mg) in N,N-dimethylformamide (2 mL) at room temperature was treated with N,N-diisopropylethyl amine (100 μL) and (benzotriazol-1-yloxytris(dimethylamino)phosphonium hexafluorophosphate (231 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 3-cyclopentyl-2-(2,3-dioxo-2,3-dihydro-indol-1-yl)-N-(1-methyl-1H-pyrazol-3-yl)-propionamide (15 mg, 12%) as a yellow solid: LCMS ESI calcd for C₂₁H₂₁N₃O₃ (M+H)⁺ 364, observed 364.

### Example 7

### 3-Cyclopentyl-N-(1-methyl-1H-pyrazol-3-yl)-2-(2-oxo-2,3-dihydro-indol-1-yl)-propionamide

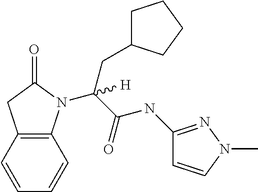

A mixture of 3-cyclopentyl-2-(2-oxo-2,3-dihydro-indol-1-yl)-propionic acid (prepared as in Example 2, 90 mg) and benzene (3 mL) at 0° C. was treated with thionyl chloride (77 μL), the reaction mixture was then heated at 80° C. for 30 min. After this time, the mixture was concentrated in vacuo to remove the benzene. The residue was then treated with a solution of 1-methyl-1H-pyrazol-3-ylamine (38 mg) in tetrahydrofuran (3 mL) and 2,6-lutidine (97 μL) and stirred at room temperature overnight. The reaction mixture was then concentrated in vacuo and the residue was dissolved in ethyl acetate and washed with a saturated brine solution. The organic layer was then dried over sodium sulfate, filtered and the filterate concentrated in vacuo and the residue was purified by column chromatography (silica gel) to afford 3-cyclopentyl-N-(1-methyl-1H-pyrazol-3-yl)-2-(2-oxo-2,3-dihydro-indol-1-yl)-propionamide (7 mg, 6%): LCMS ESI calcd for C₂₀H₂₄N₄O₂ (M+H)⁺ 353, observed 353.

### Example 8

### 3-Cyclopentyl-2-(2-oxo-2,3-dihydro-indol-1-yl)-N-pyridin-2-yl-propionamide

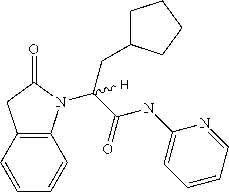

A mixture of 3-cyclopentyl-2-(2-oxo-2,3-dihydro-indol-1-yl)-propionic acid (prepared as in Example 2, 80 mg) and benzene (3 mL) was treated with thionyl chloride (29 μL) and heated at 80° C. for 2 h. After this time, the mixture was concentrated in vacuo to remove the benzene and the residue was dissolved in tetrahydrofuran (3 mL). The resulting solution was then treated with pyridin-2-ylamine (33 mg) and 2,6-lutidine (102 μL) and stirred at room temperature for 16 h. The reaction mixture was then dissolved in ethyl acetate and washed with a 1N aqueous hydrochloric acid solution and a saturated aqueous sodium bicarbonate solution. The organic layer was then dried over sodium sulfate, filtered and the filterate concentrated in vacuo and the residue was purified by biotage column chromatography (silica gel) to afford 3-cyclopentyl-2-(2-oxo-2,3-dihydro-indol-1-yl)-N-pyridin-2-yl-propionamide (12 mg, 12%): LCMS ESI calcd for C₂₁H₂₃N₃O₂ (M+H)⁺ 350, observed 350.

### Example 9

### 2-(2,3-Dioxo-2,3-dihydro-indol-1-yl)-4-methyl-pentanoic acid pyridin-2-ylamide

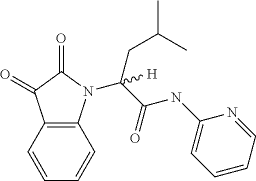

A solution of 4-methyl-pentanoic acid (23.23 g) in carbon tetrachloride (20 mL) was treated with thionyl chloride (58.0 mL) and heated at 65° C. for 30 min. The reaction mixture was then removed from the oil bath and a mixture of N-bromosuccinimide (42.8 g) and a 48% aqueous hydrobromic acid solution (20 drops) in carbon tetrachloride (100 mL) was added dropwise. The resulting mixture was then heated at 70° C. for 10 min and then 85° C. for 3 h. After this time, the reaction was cooled to 0° C. in an ice bath and then treated dropwise with methanol (50 mL). The mixture was then filtered through a celite plug to remove the insoluble material and the filterate concentrated in vacuo. The residue was then dissolved in pentane (400 mL) and washed with water (400 mL) and the aqueous layer was then extracted with pentane (400 mL). The organic layers were then combined and dried over sodium sulfate, filtered and the filterate concentrated in vacuo. The crude material was purified using flash column chromatography (silica gel, 5% ethyl acetate/hexanes) to afford 2-bromo-4-methyl-pentanoic acid methyl ester (21.75 g, 52%) as a colorless oil.

A stirred suspension of sodium hydride (39 mg) in N,N-dimethylformamide (5 mL) at 0° C. was treated with a solution of 1H-indole-2,3-dione (0.20 g) in N,N-dimethylformamide. The reaction mixture was stirred for 30 min at 0° C. and then 2-bromo-4-methyl-pentanoic acid methyl ester (341 mg) was added and it was stirred for 1 h at 0° C. It was then slowly allowed to warm to room temperature and stirred for another 3 h at room temperature. After this time, the reaction mixture was diluted with water and extracted with methylene chloride. The organic layers were combined and dried over sodium sulfate, filtered and the filterate concentrated in vacuo. The crude material was purified by column chromatography (silica gel) to afford 2-(2,3-dioxo-2,3-dihydro-indol-1-yl)-4-methyl-pentanoic acid methyl ester (250 mg, 72%).

A mixture of 2-(2,3-dioxo-2,3-dihydro-indol-1-yl)-4-methyl-pentanoic acid methyl ester (250 mg) in tetrahydrofuran:water (1:1, 20 mL) was stirred at room temperature and treated with lithium hydroxide monohydrate (76 mg). The reaction mixture was then stirred for 2 h at room temperature. After this time, the reaction mixture was concentrated in vacuo to remove the tetrahydrofuran. The remaining aqueous layer was diluted with water and then acidified to pH=2 with a 1N aqueous hydrochloric acid solution. The aqueous layer was then extracted with ethyl acetate and the organic layers combined, dried over sodium sulfate, filtered and the filterate concentrated in vacuo to afford 2-(2,3-dioxo-2,3-dihydro-indol-1-yl)-4-methyl-pentanoic acid (200 mg, 84%).

A mixture of 2-(2,3-dioxo-2,3-dihydro-indol-1-yl)-4-methyl-pentanoic acid (100 mg) and pyridin-2-ylamine (43 mg) in N,N-dimethylformamide (2 mL) at room temperature was treated with N,N-diisopropylethyl amine (200 μL) and (benzotriazol-1-yloxytris(dimethylamino)phosphonium hexafluorophosphate (243 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(2,3-dioxo-2,3-dihydro-indol-1-yl)-4-methyl-pentanoic acid pyridin-2-ylamide (10 mg, 8%) as a yellow solid: LCMS ESI calcd for C₁₉H₁₉N₃O₃ (M+H)⁺ 338, observed 416.

### Example 10

### 2-(2,3-Dioxo-2,3-dihydro-indol-1-yl)-4-methyl-pentanoic acid thiazol-2-ylamide

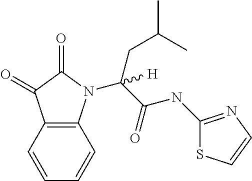

A mixture of 2-(2,3-dioxo-2,3-dihydro-indol-1-yl)-4-methyl-pentanoic acid (prepared as in Example 9, 100 mg) and thiazol-2-ylamine (46 mg) in N,N-dimethylformamide (2 mL) at room temperature was treated with N,N-diisopropylethyl amine (110 μL) and (benzotriazol-1-yloxytris(dimethylamino)phosphonium hexafluorophosphate (243 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(2,3-dioxo-2,3-dihydro-indol-1-yl)-4-methyl-pentanoic acid thiazol-2-ylamide (90 mg, 68%) as a yellow solid: LCMS ESI calcd for C₁₇H₁₇N₃O₃S (M+H)⁺ 344, observed 344.

### Example 11

### 4-Methyl-2-(2-oxo-2,3-dihydro-indol-1-yl)-pentanoic acid thiazol-2-ylamide

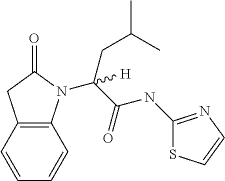

A mixture of 2-(2,3-dioxo-2,3-dihydro-indol-1-yl)-4-methyl-pentanoic acid (prepared as in Example 9, 500 mg) and hydrazine hydrate (1 mL) were placed in a sealed tube and heated at 120° C. for 25 min. The reaction mixture was then cooled to room temperature and concentrated in vacuo. The residue was dissolved in methylene chloride and washed with a 0.5N aqueous hydrochloric acid solution. The organic layer was then dried over sodium sulfate, filtered and the filterate concentrated in vacuo to afford 4-methyl-2-(2-oxo-2,3-dihydro-indol-1-yl)-pentanoic acid (400 mg, 85%).

A mixture of 4-methyl-2-(2-oxo-2,3-dihydro-indol-1-yl)-pentanoic acid (100 mg) and thiazol-2-ylamine (49 mg) in N,N-dimethylformamide (2 mL) at room temperature was treated with N,N-diisopropylethyl amine (220 μL) and (benzotriazol-1-yloxytris(dimethylamino)phosphonium hexafluorophosphate (268 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 4-methyl-2-(2-oxo-2,3-dihydro-indol-1-yl)-pentanoic acid thiazol-2-ylamide (40 mg, 30%) as a light yellow solid: LCMS ESI calcd for C₁₇H₁₉N₃O₂S (M+H)⁺ 330, observed 330.

### Example 12

### 4-Methyl-2-(2-oxo-2,3-dihydro-indol-1-yl)-pentanoic acid pyridin-2-ylamide

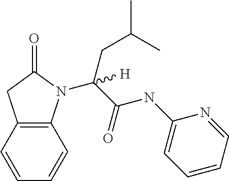

A mixture of 4-methyl-2-(2-oxo-2,3-dihydro-indol-1-yl)-pentanoic acid (prepared as in Example 11, 200 mg) and benzene (5 mL) at 0° C. was treated with thionyl chloride (72 μL), the reaction mixture was then heated at 80° C. for 30 min. After this time, the mixture was concentrated in vacuo to remove the benzene. The residue was then treated with a solution of pyridin-2-ylamine (92 mg) in tetrahydrofuran (5 mL) and 2,6-lutidine (300 μL) and stirred at room temperature overnight. The reaction mixture was then concentrated in vacuo and the residue was dissolved in ethyl acetate and washed a saturated brine solution. The organic layer was then dried over sodium sulfate, filtered and the filterate concentrated in vacuo and the residue was purified by column chromatography (silica gel) to afford 4-methyl-2-(2-oxo-2,3-dihydro-indol-1-yl)-pentanoic acid pyridin-2-ylamide (20 mg, 8%): LCMS ESI calcd for C₁₉H₂₁N₃O₂ (M+H)⁺ 324, observed 324.

### Example 13

### 2-(4-Chloro-2,3-dioxo-2,3-dihydro-indol-1-yl)-3-cyclopentyl-N-pyrazin-2-yl-propionamide

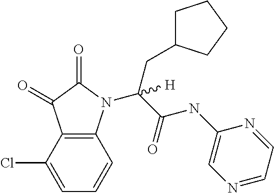

A stirred suspension of sodium hydride (60% dispersion in oil, 238 mg) in N,N-dimethylformamide (25 mL) at 0° C. was treated with a solution of (4-chloro-2,3-dioxo-2,3-dihydro-indol-1-yl)-acetic acid methyl ester (1.0 g) in N,N-dimethylformamide. The reaction mixture was stirred for 30 min at 0° C. and then 2-bromo-3-cyclopentyl-propionic acid methyl ester (prepared as in Example 1, 1.3 g) was added and it was stirred for 1 h at 0° C. It was then slowly allowed to warm to room temperature and stirred for another 12 h at room temperature. After this time, the reaction mixture was diluted with water and extracted with methylene chloride. The organic layers were combined and dried over sodium sulfate, filtered and the filterate concentrated in vacuo. The crude material was purified by column chromatography (silica gel) to afford 2-(4-chloro-2,3-dioxo-2,3-dihydro-indol-1-yl)-3-cyclopentyl-propionic acid methyl ester (1.2 g, 72%).

A mixture of 2-(4-chloro-2,3-dioxo-2,3-dihydro-indol-1-yl)-3-cyclopentyl-propionic acid methyl ester (1.2 g) and tetrahydrofuran:water (1:1, 20 mL) was stirred at room temperature and treated with lithium hydroxide monohydrate (451 mg). The reaction mixture was then stirred for 18 h at room temperature. After this time, the reaction mixture was concentrated in vacuo to remove the tetrahydrofuran. The remaining aqueous layer was diluted with water and then acidified to pH=2 with a 1N aqueous hydrochloric acid solution. The aqueous layer was then extracted with ethyl acetate (3×20 mL) and the organic layers combined, dried over sodium sulfate, filtered and the filterate concentrated in vacuo to afford 2-(4-chloro-2,3-dioxo-2,3-dihydro-indol-1-yl)-3-cyclopentyl-propionic acid (1.1 g, 96%).

A mixture of 2-(4-chloro-2,3-dioxo-2,3-dihydro-indol-1-yl)-3-cyclopentyl-propionic acid (75 mg) in methylene chloride (5 mL) and N,N-dimethylformamide (one drop) at room temperature was treated with oxalyl chloride (53 μL) and stirred for 30 min at room temperature. After this time the reaction mixture was concentrated in vacuo. The residue was dissolved in tetrahydrofuran (5 mL) and then treated with pyrazin-2-ylamine (27 mg) and 2,6-lutidine (69 μL). The resulting reaction mixture was then stirred overnight at room temperature. After this time, the reaction mixture was diluted with ethyl acetate, and washed with a 1N aqueous hydrochloric acid solution, saturated aqueous sodium bicarbonate solution and brine. The organic layers were dried over sodium sulfate, filtered and the filterate concentrated in vacuo. The crude material was purified by column chromatography (silica gel) to afford 2-(4-chloro-2,3-dioxo-2,3-dihydro-indol-1-yl)-3-cyclopentyl-N-pyrazin-2-yl-propionamide (15 mg, 16%): LCMS ESI calcd for C₂₀H₁₉ClN₄O₃ (M+H)⁺ 399, observed 399.

### Example 14

### 2-(4-Chloro-2,3-dioxo-2,3-dihydro-indol-1-yl)-3-cyclopentyl-N-pyridin-2-yl-propionamide

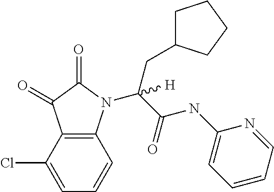

A mixture of 2-(4-chloro-2,3-dioxo-2,3-dihydro-indol-1-yl)-3-cyclopentyl-propionic acid (prepared as in Example 13, 75 mg) and pyridin-2-ylamine (26 mg) in N,N-dimethylformamide at room temperature was treated with N,N-diisopropylethyl amine (67 μL) and (benzotriazol-1-yloxytris(dimethylamino)phosphonium hexafluorophosphate (155 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(4-chloro-2,3-dioxo-2,3-dihydro-indol-1-yl)-3-cyclopentyl-N-pyridin-2-yl-propionamide (25 mg, 27%): LCMS ESI calcd for C₂₁H₂₀ClN₃O₃ (M+H)⁺ 398, observed 398.

### Example 15

### 2-(4-Chloro-2,3-dioxo-2,3-dihydro-indol-1-yl)-3-cyclopentyl-N-(1-methyl-1H-pyrazol-3-yl)-propionamide

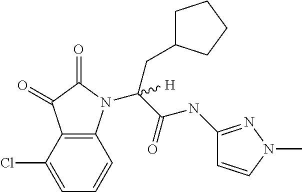

A mixture of 2-(4-chloro-2,3-dioxo-2,3-dihydro-indol-1-yl)-3-cyclopentyl-propionic acid (prepared as in Example 13, 75 mg) and 1-methyl-1H-pyrazol-3-ylamine (27 mg) in N,N-dimethylformamide at room temperature was treated with N,N-diisopropylethyl amine (67 μL) and (benzotriazol-1-yloxytris(dimethylamino)phosphonium hexafluorophosphate (155 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(4-chloro-2,3-dioxo-2,3-dihydro-indol-1-yl)-3-cyclopentyl-N-(1-methyl-1H-pyrazol-3-yl)-propionamide (70 mg, 75%): LCMS ESI calcd for C₂₀H₂₁ClN₄O₃ (M+H)⁺ 401, observed 401.

### Example 16

### 2-(4-Chloro-2,3-dioxo-2,3-dihydro-indol-1-yl)-3-cyclopentyl-N-thiazol-2-yl-propionamide

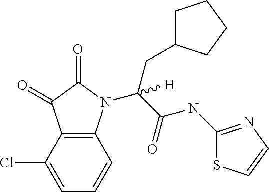

A mixture of 2-(4-chloro-2,3-dioxo-2,3-dihydro-indol-1-yl)-3-cyclopentyl-propionic acid (prepared as in Example 13, 75 mg) and thiazol-2-ylamine (28 mg) in N,N-dimethylformamide at room temperature was treated with N,N-diisopropylethyl amine (67 μL) and (benzotriazol-1-yloxytris(dimethylamino)phosphonium hexafluorophosphate (155 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(4-chloro-2,3-dioxo-2,3-dihydro-indol-1-yl)-3-cyclopentyl-N-thiazol-2-yl-propionamide (60 mg, 64%): LCMS ESI calcd for C₁₉H₁₈ClN₃O₃S (M+H)⁺ 404, observed 404.

### Example 17

### 2-(4-Chloro-2,3-dioxo-2,3-dihydro-indol-1-yl)-4-methyl-pentanoic acid pyrazin-2-ylamide

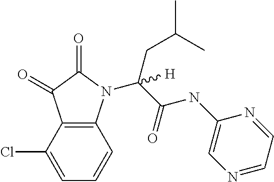

A stirred suspension of sodium hydride (60% dispersion in oil, 238 mg) in N,N-dimethylformamide (25 mL) at 0° C. was treated with a solution of (4-chloro-2,3-dioxo-2,3-dihydro-indol-1-yl)-acetic acid methyl ester (900 mg) in N,N-dimethylformamide. The reaction mixture was stirred for 30 min at 0° C. and then 2-bromo-4-methyl-pentanoic acid methyl ester (1.04 g) was added and it was stirred for 1 h at 0° C. It was then slowly allowed to warm to room temperature and stirred for another 12 h at room temperature. After this time, the reaction mixture was diluted with water and extracted with methylene chloride. The organic layers were combined and dried over sodium sulfate, filtered and the filterate concentrated in vacuo. The crude material was purified by column chromatography (silica gel) to afford 2-(4-chloro-2,3-dioxo-2,3-dihydro-indol-1-yl)-4-methyl-pentanoic acid methyl ester (1.2 g, 78%).

A mixture of 2-(4-chloro-2,3-dioxo-2,3-dihydro-indol-1-yl)-4-methyl-pentanoic acid methyl ester (1.2 g) and tetrahydrofuran:water (1:1, 20 mL) was stirred at room temperature and treated with lithium hydroxide monohydrate (489 mg). The reaction mixture was then stirred for 18 h at room temperature. After this time, the reaction mixture was concentrated in vacuo to remove the tetrahydrofuran. The remaining aqueous layer was diluted with water and then acidified to pH=2 with a 1N aqueous hydrochloric acid solution. The aqueous layer was then extracted with ethyl acetate (3×20 mL) and the organic layers combined, dried over sodium sulfate, filtered and the filterate concentrated in vacuo to afford 2-(4-chloro-2,3-dioxo-2,3-dihydro-indol-1-yl)-4-methyl-pentanoic acid (1.1 g, 96%).

A mixture of 2-(4-chloro-2,3-dioxo-2,3-dihydro-indol-1-yl)-4-methyl-pentanoic acid (75 mg) in methylene chloride (5 mL) and N,N-dimethylformamide (one drop) at room temperature was treated with oxalyl chloride (57 μL) and stirred for 30 min at room temperature. After this time the reaction mixture was concentrated in vacuo. The residue was dissolved in tetrahydrofuran (5 mL) and then treated with pyrazin-2-ylamine (29 mg) and 2,6-lutidine (75 μL). The resulting reaction mixture was then stirred overnight at room temperature. After this time, the reaction mixture was diluted with ethyl acetate, and washed with a 1N aqueous hydrochloric acid solution, saturated aqueous sodium bicarbonate solution and brine. The organic layers were dried over sodium sulfate, filtered and the filterate concentrated in vacuo. The crude material was purified by column chromatography (silica gel) to afford 2-(4-chloro-2,3-dioxo-2,3-dihydro-indol-1-yl)-4-methyl-pentanoic acid pyrazin-2-ylamide (30 mg, 31%): LCMS ESI calcd for C₁₈H₁₇ClN₄O₃ (M+H)⁺ 373, observed 373.

### Example 18

### 2-(4-Chloro-2,3-dioxo-2,3-dihydro-indol-1-yl)-4-methyl-pentanoic acid pyridin-2-ylamide

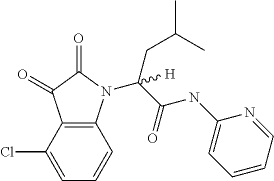

A mixture of 2-(4-chloro-2,3-dioxo-2,3-dihydro-indol-1-yl)-4-methyl-pentanoic acid (prepared as in Example 17, 75 mg) and pyridin-2-ylamine (29 mg) in N,N-dimethylformamide at room temperature was treated with N,N-diisopropylethyl amine (73 μL) and (benzotriazol-1-yloxytris(dimethylamino)phosphonium hexafluorophosphate (169 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(4-chloro-2,3-dioxo-2,3-dihydro-indol-1-yl)-4-methyl-pentanoic acid pyridin-2-ylamide (15 mg, 16%): LCMS ESI calcd for C₁₉H₁₈ClN₃O₃ (M+H)⁺ 372, observed 372.

### Example 19

### 2-(4-Chloro-2,3-dioxo-2,3-dihydro-indol-1-yl)-4-methyl-pentanoic acid (1-methyl-1H-pyrazol-3-yl)-amide

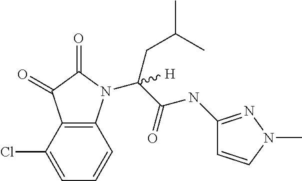

A mixture of 2-(4-chloro-2,3-dioxo-2,3-dihydro-indol-1-yl)-4-methyl-pentanoic acid (prepared as in Example 17, 75 mg) and 1-methyl-1H-pyrazol-3-ylamine (30 mg) in N,N-dimethylformamide at room temperature was treated with N,N-diisopropylethyl amine (73 μL) and (benzotriazol-1-yloxytris(dimethylamino)phosphonium hexafluorophosphate (169 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(4-chloro-2,3-dioxo-2,3-dihydro-indol-1-yl)-4-methyl-pentanoic acid (1-methyl-1H-pyrazol-3-yl)-amide (50 mg, 53%): LCMS ESI calcd for C₁₈H₁₉ClN₄O₃ (M+H)⁺ 375, observed 375.

### Example 20

### 2-(4-Chloro-2,3-dioxo-2,3-dihydro-indol-1-yl)-4-methyl-pentanoic acid thiazol-2-ylamide

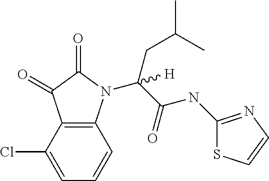

A mixture of 2-(4-chloro-2,3-dioxo-2,3-dihydro-indol-1-yl)-4-methyl-pentanoic acid (prepared as in Example 17, 75 mg) and thiazol-2-ylamine (31 mg) in N,N-dimethylformamide at room temperature was treated with N,N-diisopropylethyl amine (73 μL) and (benzotriazol-1-yloxytris(dimethylamino)phosphonium hexafluorophosphate (169 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(4-chloro-2,3-dioxo-2,3-dihydro-indol-1-yl)-4-methyl-pentanoic acid thiazol-2-ylamide (60 mg, 63%): LCMS ESI calcd for C₁₇H₁₆ClN₃O₃S (M+H)⁺ 378, observed 378.

### Example 21

### 2-(4-Chloro-2-oxo-2,3-dihydro-indol-1-yl)-3-cyclopentyl-N-pyridin-2-yl-propionamide

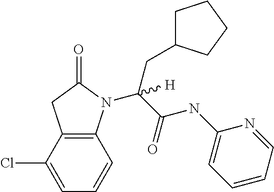

A mixture of 2-(4-chloro-2,3-dioxo-2,3-dihydro-indol-1-yl)-3-cyclopentyl-propionic acid (prepared as in Example 13, 400 mg) and hydrazine hydrate (1 mL) were placed in a sealed tube and heated at 120° C. for 15 min. The reaction mixture was then cooled to room temperature and concentrated in vacuo. The residue was dissolved in methylene chloride (30 mL) and washed with a 0.5N aqueous hydrochloric acid solution and a saturated aqueous brine solution. The organic layer was then dried over sodium sulfate, filtered and the filterate concentrated in vacuo to afford 2-(4-chloro-2-oxo-2,3-dihydro-indol-1-yl)-3-cyclopentyl-propionic acid (360 mg, 94%).

A mixture of 2-(4-chloro-2-oxo-2,3-dihydro-indol-1-yl)-3-cyclopentyl-propionic acid (65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with pyridin-2-ylamine (24 mg) and 2,6-lutidine (74 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(4-chloro-2-oxo-2,3-dihydro-indol-1-yl)-3-cyclopentyl-N-pyridin-2-yl-propionamide (20 mg, 25%) as an off-white solid: LCMS ESI calcd for C₂₁H₂₂ClN₃O₂ (M+H)⁺ 384, observed 384.

### Example 22

### 2-(4-Chloro-2-oxo-2,3-dihydro-indol-1-yl)-3-cyclopentyl-N-thiazol-2-yl-propionamide

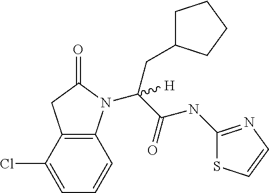

A mixture of 2-(4-chloro-2-oxo-2,3-dihydro-indol-1-yl)-3-cyclopentyl-propionic acid (prepared as in Example 21, 65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with thiazol-2-ylamine (25 mg) and 2,6-lutidine (74 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(4-chloro-2-oxo-2,3-dihydro-indol-1-yl)-3-cyclopentyl-N-thiazol-2-yl-propionamide (58 mg, 71%) as an off-white solid: LCMS ESI calcd for C₁₉H₂₀ClN₃O₂S (M+H)⁺ 390, observed 390.

### Example 23

### 2-(4-Chloro-2-oxo-2,3-dihydro-indol-1-yl)-3-cyclopentyl-N-(1-methyl-1H-pyrazol-3-yl)-propionamide

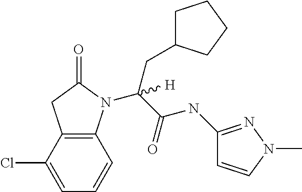

A mixture of 2-(4-chloro-2-oxo-2,3-dihydro-indol-1-yl)-3-cyclopentyl-propionic acid (prepared as in Example 21, 65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with 1-methyl-1H-pyrazol-3-ylamine (25 mg) and 2,6-lutidine (74 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(4-chloro-2-oxo-2,3-dihydro-indol-1-yl)-3-cyclopentyl-N-(1-methyl-1H-pyrazol-3-yl)-propionamide (65 mg, 80%) as an off-white solid: LCMS ESI calcd for C₂₀H₂₃ClN₄O₂ (M+H)⁺ 387, observed 387.

### Example 24

### 2-(4-Chloro-2-oxo-2,3-dihydro-indol-1-yl)-3-cyclopentyl-N-pyrazin-2-yl-propionamide

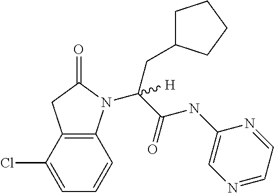

A mixture of 2-(4-chloro-2-oxo-2,3-dihydro-indol-1-yl)-3-cyclopentyl-propionic acid (prepared as in Example 21, 65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with pyrazin-2-ylamine (24 mg) and 2,6-lutidine (74 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(4-chloro-2-oxo-2,3-dihydro-indol-1-yl)-3-cyclopentyl-N-pyrazin-2-yl-propionamide (25 mg, 31%) as an off-white solid: LCMS ESI calcd for C₂₀H₂₁ClN₄O₂ (M+H)⁺ 385, observed 385.

### Example 25

### 2-(4-Chloro-2-oxo-2,3-dihydro-indol-1-yl)-4-methyl-pentanoic acid pyrazin-2-ylamide

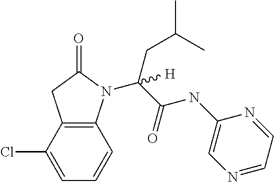

A mixture of 2-(4-chloro-2,3-dioxo-2,3-dihydro-indol-1-yl)-4-methyl-pentanoic acid (prepared as in Example 17, 450 mg) and hydrazine hydrate (1 mL) were placed in a sealed tube and heated at 120° C. for 15 min. The reaction mixture was then cooled to room temperature and concentrated in vacuo. The residue was dissolved in methylene chloride (30 mL) and washed with a 0.5N aqueous hydrochloric acid solution and a saturated aqueous brine solution. The organic layer was then dried over sodium sulfate, filtered and the filterate concentrated in vacuo to afford 2-(4-chloro-2-oxo-2,3-dihydro-indol-1-yl)-4-methyl-pentanoic acid (410 mg, 96%).

A mixture of 2-(4-chloro-2-oxo-2,3-dihydro-indol-1-yl)-4-methyl-pentanoic acid (65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with pyrazin-2-ylamine (26 mg) and 2,6-lutidine (81 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(4-chloro-2-oxo-2,3-dihydro-indol-1-yl)-4-methyl-pentanoic acid pyrazin-2-ylamide (20 mg, 25%) as a yellow solid: LCMS ESI calcd for C₁₈H₁₉ClN₄O₂ (M+H)⁺ 359, observed 359.

### Example 26

### 2-(4-Chloro-2-oxo-2,3-dihydro-indol-1-yl)-4-methyl-pentanoic acid pyridin-2-ylamide

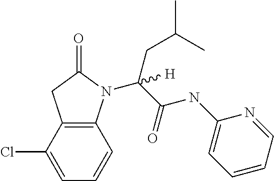

A mixture of 2-(4-chloro-2-oxo-2,3-dihydro-indol-1-yl)-4-methyl-pentanoic acid (prepared as in Example 25, 65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with pyridin-2-ylamine (26 mg) and 2,6-lutidine (81 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(4-chloro-2-oxo-2,3-dihydro-indol-1-yl)-4-methyl-pentanoic acid pyridin-2-ylamide (13 mg, 16%) as a yellow solid: LCMS ESI calcd for C₁₉H₂₀ClN₃O₂ (M+H)⁺ 358, observed 358.

### Example 27

### 2-(4-Chloro-2-oxo-2,3-dihydro-indol-1-yl)-4-methyl-pentanoic acid (1-methyl-1H-pyrazol-3-yl)-amide

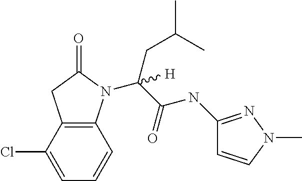

A mixture of 2-(4-chloro-2-oxo-2,3-dihydro-indol-1-yl)-4-methyl-pentanoic acid (prepared as in Example 25, 65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with 1-methyl-1H-pyrazol-3-ylamine (27 mg) and 2,6-lutidine (81 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(4-chloro-2-oxo-2,3-dihydro-indol-1-yl)-4-methyl-pentanoic acid (1-methyl-1H-pyrazol-3-yl)-amide (33 mg, 40%) as a yellow solid: LCMS ESI calcd for C₁₈H₂₁ClN₄O₂ (M+H)⁺ 361, observed 361.

### Example 28

### 2-(4-Chloro-2-oxo-2,3-dihydro-indol-1-yl)-4-methyl-pentanoic acid thiazol-2-ylamide

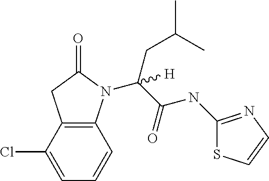

A mixture of 2-(4-chloro-2-oxo-2,3-dihydro-indol-1-yl)-4-methyl-pentanoic acid (prepared as in Example 25, 65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with thiazol-2-ylamine (28 mg) and 2,6-lutidine (81 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(4-chloro-2-oxo-2,3-dihydro-indol-1-yl)-4-methyl-pentanoic acid thiazol-2-ylamide (39 mg, 46%) as a yellow solid: LCMS ESI calcd for C₁₇H₁₈ClN₃O₂S (M+H)⁺ 364, observed 364.

### Example 29

### 3-Cyclopentyl-2-(2,3-dioxo-5-trifluoromethoxy-2,3-dihydro-indol-1-yl)-N-pyrazin-2-yl-propionamide

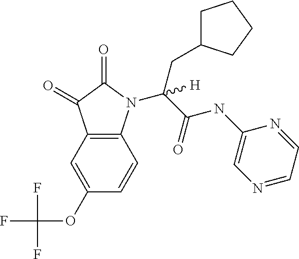

A stirred suspension of sodium hydride (60% dispersion in oil, 208 mg) in N,N-dimethylformamide (20 mL) at 0° C. was treated with a solution of 5-trifluoromethoxy-1H-indole-2,3-dione (1.0 g) in N,N-dimethylformamide. The reaction mixture was stirred for 30 min at 0° C. and then 2-bromo-3-cyclopentyl-propionic acid methyl ester (prepared as in Example 1, 1.22 g) was added and it was stirred for 1 h at 0° C. It was then slowly allowed to warm to room temperature and stirred for another 3 h at room temperature. After this time, the reaction mixture was diluted with water and extracted with methylene chloride. The organic layers were combined and dried over sodium sulfate, filtered and the filterate concentrated in vacuo. The crude material was purified by column chromatography (silica gel) to afford 3-cyclopentyl-2-(2,3-dioxo-5-trifluoromethoxy-2,3-dihydro-indol-1-yl)-propionic acid methyl ester (1.0 g, 60%).

A mixture of 3-cyclopentyl-2-(2,3-dioxo-5-trifluoromethoxy-2,3-dihydro-indol-1-yl)-propionic acid methyl ester (1.0 g) and tetrahydrofuran:water (1:1, 20 mL) was stirred at room temperature and treated with lithium hydroxide monohydrate (218 mg). The reaction mixture was then stirred for 2 h at room temperature. After this time, the reaction mixture was concentrated in vacuo to remove the tetrahydrofuran. The remaining aqueous layer was diluted with water and then acidified to pH=2 with a 1N aqueous hydrochloric acid solution. The aqueous layer was then extracted with ethyl acetate and the organic layers combined, dried over sodium sulfate, filtered and the filterate concentrated in vacuo to afford 3-cyclopentyl-2-(2,3-dioxo-5-trifluoromethoxy-2,3-dihydro-indol-1-yl)-propionic acid (900 mg, 93%).

A mixture of 3-cyclopentyl-2-(2,3-dioxo-5-trifluoromethoxy-2,3-dihydro-indol-1-yl)-propionic acid (50 mg) in methylene chloride (5 mL) was cooled to 0° C. and treated with oxalyl chloride (14 μL) followed by N,N-dimethylformamide (one drop) and stirred for 30 min at 0° C. After this time the reaction mixture was concentrated in vacuo. The residue was dissolved in tetrahydrofuran (5 mL) and then treated with pyrazin-2-ylamine (15 mg) and 2,6-lutidine (47 μL). The resulting reaction mixture was then stirred at room temperature for 16 h. After this time, the reaction mixture was concentrated in vacuo and the residue dissolved in ethyl acetate (10 mL), and washed with a 0.5N aqueous hydrochloric acid solution (2×3 mL), saturated aqueous sodium bicarbonate solution (2×3 mL) and brine (4 mL). The organic layers were dried over sodium sulfate, filtered and the filterate concentrated in vacuo. The crude material was purified by Biotage column chromatography (silica gel, 15% ethyl acetate/hexanes) to afford semi-pure material (20 mg). The coupling reaction was performed again on the same scale and the 20 mg of material from the first run was combined with this and it was purified again as one batch using Biotage column chromatography (silica gel, 15% ethyl acetate/hexanes) to afford 3-cyclopentyl-2-(2,3-dioxo-5-trifluoromethoxy-2,3-dihydro-indol-1-yl)-N-pyrazin-2-yl-propionamide (50 mg, 42%, from the two combined coupling reactions): LCMS ESI calcd for C₂₁H₁₉F₃N₄O₄ (M+H)⁺ 449, observed 449.

### Example 30

### 3-Cyclopentyl-2-(2,3-dioxo-5-trifluoromethoxy-2,3-dihydro-indol-1-yl)-N-pyridin-2-yl-propionamide

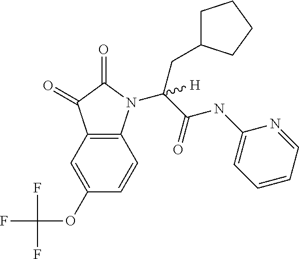

A mixture of 3-cyclopentyl-2-(2,3-dioxo-5-trifluoromethoxy-2,3-dihydro-indol-1-yl)-propionic acid (prepared as in Example 29, 75 mg) and pyridin-2-ylamine (23 mg) in N,N-dimethylformamide (2 mL) at room temperature was treated with N,N-diisopropylethyl amine (100 μL) and (benzotriazol-1-yloxytris(dimethylamino)phosphonium hexafluorophosphate (135 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 3-cyclopentyl-2-(2,3-dioxo-5-trifluoromethoxy-2,3-dihydro-indol-1-yl)-N-pyridin-2-yl-propionamide (15 mg, 17%) as an orange solid: LCMS ESI calcd for C₂₂H₂₀F₃N₃O₄ (M+H)⁺ 448, observed 448.

### Example 31

### 3-Cyclopentyl-2-(2,3-dioxo-5-trifluoromethoxy-2,3-dihydro-indol-1-yl)-N-(1-methyl-1H-pyrazol-3-yl)-propionamide

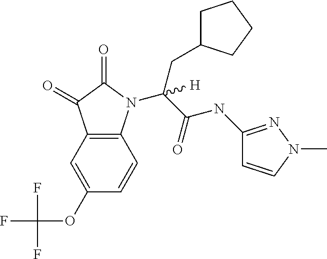

A mixture of 3-cyclopentyl-2-(2,3-dioxo-5-trifluoromethoxy-2,3-dihydro-indol-1-yl)-propionic acid (prepared as in Example 29, 75 mg) and 1-methyl-1H-pyrazol-3-ylamine (24 mg) in N,N-dimethylformamide (2 mL) at room temperature was treated with N,N-diisopropylethyl amine (100 μL) and (benzotriazol-1-yloxytris(dimethylamino)phosphonium hexafluorophosphate (135 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 3-cyclopentyl-2-(2,3-dioxo-5-trifluoromethoxy-2,3-dihydro-indol-1-yl)-N-(1-methyl-1H-pyrazol-3-yl)-propionamide (60 mg, 66%) as an orange solid: LCMS ESI calcd for C₂₁H₂₁F₃N₄O₄ (M+H)⁺ 451, observed 451.

### Example 32

### 3-Cyclopentyl-2-(2,3-dioxo-5-trifluoromethoxy-2,3-dihydro-indol-1-yl)-N-thiazol-2-yl-propionamide

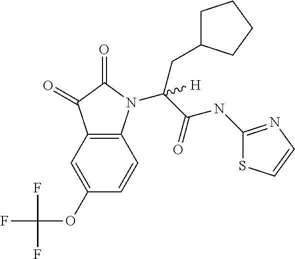

A mixture of 3-cyclopentyl-2-(2,3-dioxo-5-trifluoromethoxy-2,3-dihydro-indol-1-yl)-propionic acid (prepared as in Example 29, 75 mg) and thiazol-2-ylamine (24 mg) in N,N-dimethylformamide (2 mL) at room temperature was treated with N,N-diisopropylethyl amine (100 μL) and (benzotriazol-1-yloxytris(dimethylamino)phosphonium hexafluorophosphate (135 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 3-cyclopentyl-2-(2,3-dioxo-5-trifluoromethoxy-2,3-dihydro-indol-1-yl)-N-thiazol-2-yl-propionamide (45 mg, 49%) as an orange solid: LCMS ESI calcd for C₂₀H₁₈F₃N₃O₄S (M+H)⁺ 454, observed 454.

### Example 33

### 3-Cyclopentyl-2-(2-oxo-5-trifluoromethoxy-2,3-dihydro-indol-1-yl)-N-pyridin-2-yl-propionamide

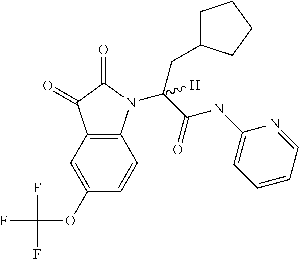

A mixture of 3-cyclopentyl-2-(2,3-dioxo-5-trifluoromethoxy-2,3-dihydro-indol-1-yl)-propionic acid (prepared as in Example 29, 400 mg) and hydrazine hydrate (1 mL) were placed in a sealed tube and heated at 120° C. for 15 min. The reaction mixture was then cooled to room temperature and concentrated in vacuo. The residue was dissolved in methylene chloride (30 mL) and washed with a 0.5N aqueous hydrochloric acid solution (2×10 mL) and a saturated aqueous brine solution (10 mL). The organic layer was then dried over sodium sulfate, filtered and the filterate concentrated in vacuo to afford 3-cyclopentyl-2-(2-oxo-5-trifluoromethoxy-2,3-dihydro-indol-1-yl)-propionic acid (356 mg, 92%).

A mixture of 3-cyclopentyl-2-(2-oxo-5-trifluoromethoxy-2,3-dihydro-indol-1-yl)-propionic acid (150 mg) and benzene (5 mL) was treated with thionyl chloride (37 μL) and heated at 80° C. for 2 h. After this time, the mixture was concentrated in vacuo to remove the benzene and the residue was dissolved in tetrahydrofuran (5 mL). The resulting solution was then treated with pyridin-2-ylamine (47 mg) and 2,6-lutidine (147 μL) and stirred at room temperature for 16 h. The reaction mixture was then concentrated in vacuo to remove the tetrahydrofuran and the residue was dissolved in ethyl acetate and washed with a 1 N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(2-oxo-5-trifluoromethoxy-2,3-dihydro-indol-1-yl)-N-pyridin-2-yl-propionamide (64 mg, 35%): LCMS ESI calcd for C₂₂H₂₂F₃N₃O₃ (M+H)⁺ 434, observed 434.

### Example 34

### 3-Cyclopentyl-N-(1-methyl-1H-pyrazol-3-yl)-2-(2-oxo-5-trifluoromethoxy-2,3-dihydro-indol-1-yl)-propionamide

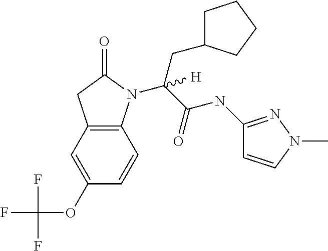

A mixture of 3-cyclopentyl-2-(2-oxo-5-trifluoromethoxy-2,3-dihydro-indol-1-yl)-propionic acid (prepared as in Example 33, 65 mg) and benzene (3 mL) was treated with thionyl chloride (18 μL, and heated at 80° C. for 2 h. After this time, the mixture was concentrated in vacuo to remove the benzene and the residue was dissolved in tetrahydrofuran (3 mL). The resulting solution was then treated with 1-methyl-1H-pyrazol-3-ylamine (21 mg) and 2,6-lutidine (65 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-N-(1-methyl-1H-pyrazol-3-yl)-2-(2-oxo-5-trifluoromethoxy-2,3-dihydro-indol-1-yl)-propionamide (23 mg, 29%) as an orange solid: LCMS ESI calcd for C₂₁H₂₃F₃N₄O₃ (M+H)⁺ 437, observed 437.

### Example 35

### 3-Cyclopentyl-2-(2-oxo-5-trifluoromethoxy-2,3-dihydro-indol-1-yl)-N-thiazol-2-yl-propionamide

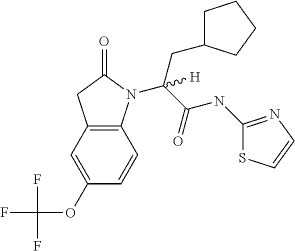

A mixture of 3-cyclopentyl-2-(2-oxo-5-trifluoromethoxy-2,3-dihydro-indol-1-yl)-propionic acid (prepared as in Example 33, 65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with thiazol-2-ylamine (22 mg) and 2,6-lutidine (65 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(2-oxo-5-trifluoromethoxy-2,3-dihydro-indol-1-yl)-N-thiazol-2-yl-propionamide (15 mg, 19%) as an orange sticky solid: LCMS ESI calcd for C₂₀H₂F₃N₃O₃S (M+H)⁺ 440, observed 440.

### Example 36

### 3-Cyclopentyl-2-(2-oxo-5-trifluoromethoxy-2,3-dihydro-indol-1-yl)-N-pyrazin-2-yl-propionamide

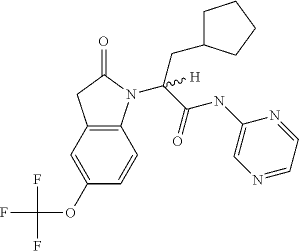

A mixture of 3-cyclopentyl-2-(2-oxo-5-trifluoromethoxy-2,3-dihydro-indol-1-yl)-propionic acid (prepared as in Example 33, 65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with pyrazin-2-ylamine (21 mg) and 2,6-lutidine (65 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(2-oxo-5-trifluoromethoxy-2,3-dihydro-indol-1-yl)-N-pyrazin-2-yl-propionamide (9 mg, 11%) as an orange sticky solid: LCMS ESI calcd for C₂₁H₂₁F₃N₄O₃ (M+H)⁺ 435, observed 435.

### Example 37

### 2-(2,3-Dioxo-5-trifluoromethoxy-2,3-dihydro-indol-1-yl)-4-methyl-pentanoic acid pyrazin-2-ylamide

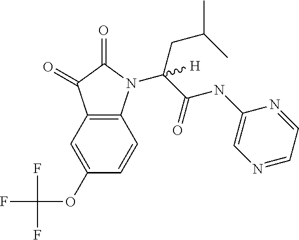

A stirred suspension of sodium hydride (60% dispersion in oil, 208 mg) in N,N-dimethylformamide (25 mL) at 0° C. was treated with a solution of 5-trifluoromethoxy-1H-indole-2,3-dione (1.0 g) in N,N-dimethylformamide. The reaction mixture was stirred for 30 min at 0° C. and then 2-bromo-4-methyl-pentanoic acid methyl ester (prepared as in Example 9, 1.1 g) was added and it was stirred for 1 h at 0° C. It was then slowly allowed to warm to room temperature and stirred for another 3 h at room temperature. After this time, the reaction mixture was diluted with water and extracted with methylene chloride. The organic layers were combined and dried over sodium sulfate, filtered and the filterate concentrated in vacuo. The crude material was purified by column chromatography (silica gel) to afford 2-(2,3-dioxo-5-trifluoromethoxy-2,3-dihydro-indol-1-yl)-4-methyl-pentanoic acid methyl ester (1.0 g, 64%).

A mixture of 2-(2,3-dioxo-5-trifluoromethoxy-2,3-dihydro-indol-1-yl)-4-methyl-pentanoic acid methyl ester (1.0 g) and tetrahydrofuran:water (1:1, 20 mL) was stirred at room temperature and treated with lithium hydroxide monohydrate (351 mg). The reaction mixture was then stirred for 2 h at room temperature. After this time, the reaction mixture was concentrated in vacuo to remove the tetrahydrofuran. The remaining aqueous layer was diluted with water and then acidified to pH=2 with a 1N aqueous hydrochloric acid solution. The aqueous layer was then extracted with ethyl acetate and the organic layers combined, dried over sodium sulfate, filtered and the filterate concentrated in vacuo to afford 2-(2,3-dioxo-5-trifluoromethoxy-2,3-dihydro-indol-1-yl)-4-methyl-pentanoic acid (1.0 g, 97%).

A mixture of 2-(2,3-dioxo-5-trifluoromethoxy-2,3-dihydro-indol-1-yl)-4-methyl-pentanoic acid (50 mg) in methylene chloride (3 mL) was cooled to 0° C. and treated with oxalyl chloride (15 μL) followed by N,N-dimethylformamide (one drop) and stirred for 30 min at 0° C. After this time the reaction mixture was concentrated in vacuo. The residue was dissolved in tetrahydrofuran (3 mL) and then treated with pyrazin-2-ylamine (17 mg) and 2,6-lutidine (51 μL). The resulting reaction mixture was then stirred at room temperature for 16 h. After this time, the reaction mixture was concentrated in vacuo and the residue dissolved in ethyl acetate (10 mL), and washed with a 0.5N aqueous hydrochloric acid solution (2×3 mL), saturated aqueous sodium bicarbonate solution (2×3 mL) and brine (4 mL). The organic layers were dried over sodium sulfate, filtered and the filterate concentrated in vacuo. The crude material was purified by Biotage column chromatography (silica gel, 15% ethyl acetate/hexanes) to afford 2-(2,3-dioxo-5-trifluoromethoxy-2,3-dihydro-indol-1-yl)-4-methyl-pentanoic acid pyrazin-2-ylamide (40 mg, 49%): LCMS ESI calcd for C₁₉H₁₇F₃N₄O₄ (M+H)⁺ 423, observed 423.

### Example 38

### 2-(2,3-Dioxo-5-trifluoromethoxy-2,3-dihydro-indol-1-yl)-4-methyl-pentanoic acid pyridin-2-ylamide

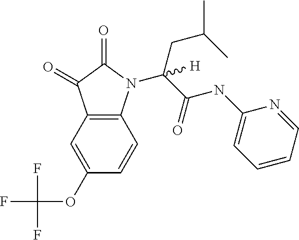

A mixture of 2-(2,3-dioxo-5-trifluoromethoxy-2,3-dihydro-indol-1-yl)-4-methyl-pentanoic acid (prepared as in Example 37, 75 mg) and pyridin-2-ylamine (25 mg) in N,N-dimethylformamide (2 mL) at room temperature was treated with N,N-diisopropylethyl amine (31 μL) and (benzotriazol-1-yloxytris(dimethylamino)phosphonium hexafluorophosphate (288 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(2,3-dioxo-5-trifluoromethoxy-2,3-dihydro-indol-1-yl)-4-methyl-pentanoic acid pyridin-2-ylamide (55 mg, 60%) as an orange solid: LCMS ESI calcd for C₂₀H₁₈F₃N₃O₄ (M+H)⁺ 422, observed 422.

### Example 39

### 2-(2,3-Dioxo-5-trifluoromethoxy-2,3-dihydro-indol-1-yl)-4-methyl-pentanoic acid (1-methyl-1H-pyrazol-3-yl)-amide

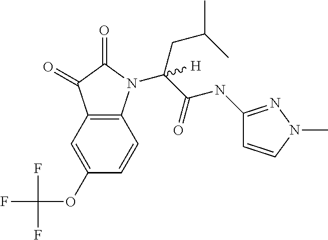

A mixture of 2-(2,3-dioxo-5-trifluoromethoxy-2,3-dihydro-indol-1-yl)-4-methyl-pentanoic acid (prepared as in Example 37, 75 mg) and 1-methyl-1H-pyrazol-3-ylamine (25 mg) in N,N-dimethylformamide (2 mL) at room temperature was treated with N,N-diisopropylethyl amine (31 μL) and (benzotriazol-1-yloxytris(dimethylamino)phosphonium hexafluorophosphate (288 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(2,3-dioxo-5-trifluoromethoxy-2,3-dihydro-indol-1-yl)-4-methyl-pentanoic acid (1-methyl-1H-pyrazol-3-yl)-amide (40 mg, 43%) as an orange solid: LCMS ESI calcd for C₁₉H₁₉F₃N₄O₄ (M+H)⁺ 425, observed 425.

### Example 40

### 2-(2,3-Dioxo-5-trifluoromethoxy-2,3-dihydro-indol-1-yl)-4-methyl-pentanoic acid thiazol-2-ylamide

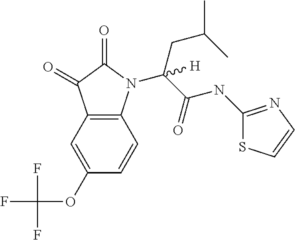

A mixture of 2-(2,3-dioxo-5-trifluoromethoxy-2,3-dihydro-indol-1-yl)-4-methyl-pentanoic acid (prepared as in Example 37, 75 mg) and thiazol-2-ylamine (26 mg) in N,N-dimethylformamide (2 mL) at room temperature was treated with N,N-diisopropylethyl amine (31 μL) and (benzotriazol-1-yloxytris(dimethylamino)phosphonium hexafluorophosphate (288 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(2,3-dioxo-5-trifluoromethoxy-2,3-dihydro-indol-1-yl)-4-methyl-pentanoic acid thiazol-2-ylamide (40 mg, 43%) as an orange solid: LCMS ESI calcd for C₁₈H₁₆F₃N₃O₄S (M+H)⁺ 428, observed 428.

### Example 41

### 4-Methyl-2-(2-oxo-5-trifluoromethoxy-2,3-dihydro-indol-1-yl)-pentanoic acid thiazol-2-ylamide

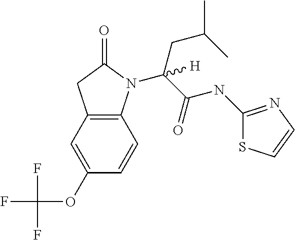

A mixture of 2-(2,3-dioxo-5-trifluoromethoxy-2,3-dihydro-indol-1-yl)-4-methyl-pentanoic acid (prepared as in Example 37, 400 mg) and hydrazine hydrate (1 mL) were placed in a sealed tube and heated at 120° C. for 15 min. The reaction mixture was then cooled to room temperature and concentrated in vacuo. The residue was dissolved in methylene chloride (30 mL) and washed with a 0.5N aqueous hydrochloric acid solution (2×10 mL) and a saturated aqueous brine solution (10 mL). The organic layer was then dried over sodium sulfate, filtered and the filterate concentrated in vacuo to afford 4-methyl-2-(2-oxo-5-trifluoromethoxy-2,3-dihydro-indol-1-yl)-pentanoic acid (360 mg, 93%).

A mixture of 4-methyl-2-(2-oxo-5-trifluoromethoxy-2,3-dihydro-indol-1-yl)-pentanoic acid (150 mg) and benzene (5 mL) at 0° C. was treated with thionyl chloride (40 μL) and then heated at 80° C. for 30 min. After this time, the mixture was concentrated in vacuo to remove the benzene. The residue was then treated with a solution of thiazol-2-ylamine (54 mg) and 2,6-lutidine (200 μL) in tetrahydrofuran (5 mL) and stirred at room temperature overnight. The reaction mixture was then concentrated in vacuo to remove the tetrahydrofuran and the residue was dissolved in ethyl acetate and washed with a saturated aqueous brine solution. The organic layer was then dried over sodium sulfate, filtered and the filterate concentrated in vacuo and the residue was purified by column chromatography (silica gel) to afford 4-methyl-2-(2-oxo-5-trifluoromethoxy-2,3-dihydro-indol-1-yl)-pentanoic acid thiazol-2-ylamide (58 mg, 81%): LCMS ESI calcd for C₁₈H₁₈F₃N₃O₃S (M+H)⁺ 414, observed 414.

### Example 42

### 4-Methyl-2-(2-oxo-5-trifluoromethoxy-2,3-dihydro-indol-1-yl)-pentanoic acid (1-methyl-1H-pyrazol-3-yl)-amide

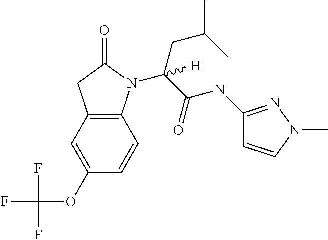

A mixture of 4-methyl-2-(2-oxo-5-trifluoromethoxy-2,3-dihydro-indol-1-yl)-pentanoic acid (prepared as in Example 41, 60 mg) and benzene (3 mL) was treated with thionyl chloride (17 μL) and heated at 80° C. for 2 h. After this time, the mixture was concentrated in vacuo to remove the benzene and the residue was dissolved in tetrahydrofuran (3 mL). The resulting solution was then treated with 1-methyl-1H-pyrazol-3-ylamine (21 mg) and 2,6-lutidine (60 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 4-methyl-2-(2-oxo-5-trifluoromethoxy-2,3-dihydro-indol-1-yl)-pentanoic acid (1-methyl-1H-pyrazol-3-yl)-amide (50 mg, 67%) as an orange solid: LCMS ESI calcd for C₁₉H₂₁F₃N₄O₃ (M+H)⁺ 411, observed 411.

### Example 43

### 4-Methyl-2-(2-oxo-5-trifluoromethoxy-2,3-dihydro-indol-1-yl)-pentanoic acid pyridin-2-ylamide

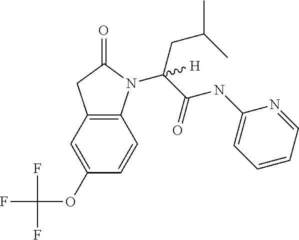

A mixture of 4-methyl-2-(2-oxo-5-trifluoromethoxy-2,3-dihydro-indol-1-yl)-pentanoic acid (prepared as in Example 41, 60 mg) and benzene (3 mL) was treated with thionyl chloride (17 μL) and heated at 80° C. for 2 h. After this time, the mixture was concentrated in vacuo to remove the benzene and the residue was dissolved in tetrahydrofuran (3 mL). The resulting solution was then treated with pyridin-2-ylamine (20 mg) and 2,6-lutidine (60 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 4-methyl-2-(2-oxo-5-trifluoromethoxy-2,3-dihydro-indol-1-yl)-pentanoic acid pyridin-2-ylamide (37 mg, 50%) as an off-white solid: LCMS ESI calcd for C₂₀H₂₀F₃N₃O₃ (M+H)⁺ 408, observed 408.

### Example 44

### 4-Methyl-2-(2-oxo-5-trifluoromethoxy-2,3-dihydro-indol-1-yl)-pentanoic acid pyrazin-2-ylamide

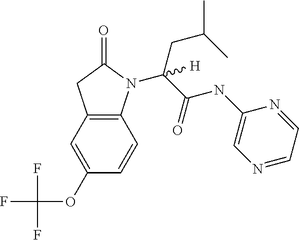

A mixture of 4-methyl-2-(2-oxo-5-trifluoromethoxy-2,3-dihydro-indol-1-yl)-pentanoic acid (prepared as in Example 41, 60 mg) and benzene (3 mL) was treated with thionyl chloride (17 μL) and heated at 80° C. for 2 h. After this time, the mixture was concentrated in vacuo to remove the benzene and the residue was dissolved in tetrahydrofuran (3 mL). The resulting solution was then treated with pyrazin-2-ylamine (21 mg) and 2,6-lutidine (60 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 4-methyl-2-(2-oxo-5-trifluoromethoxy-2,3-dihydro-indol-1-yl)-pentanoic acid pyrazin-2-ylamide (28 mg, 38%) as an orange sticky solid: LCMS ESI calcd for C₁₉H₁₉F₃N₄O₃ (M+H)⁺ 409, observed 409.

### Example 45

### 3-Cyclopentyl-2-(5-methoxy-2,3-dioxo-2,3-dihydro-indol-1-yl)-N-pyrazin-2-yl-propionamide

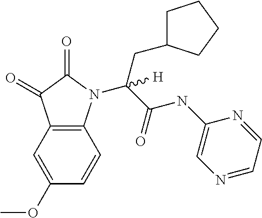

A stirred suspension of sodium hydride (60% dispersion in oil, 271 mg) in N,N-dimethylformamide (25 mL) at 0° C. was treated with a solution of 5-methoxy-1H-indole-2,3-dione (1.0 g) in N,N-dimethylformamide. The reaction mixture was stirred for 30 min at 0° C. and then 2-bromo-3-cyclopentyl-propionic acid methyl ester (prepared as in Example 1, 1.6 g) was added and it was stirred for 1 h at 0° C. It was then slowly allowed to warm to room temperature and stirred for another 3 h at room temperature. After this time, the reaction mixture was diluted with water and extracted with methylene chloride. The organic layers were combined and dried over sodium sulfate, filtered and the filterate concentrated in vacuo. The crude material was purified by column chromatography (silica gel) to afford 3-cyclopentyl-2-(5-methoxy-2,3-dioxo-2,3-dihydro-indol-1-yl)-propionic acid methyl ester (1.5 g, 80%).

A mixture of 3-cyclopentyl-2-(5-methoxy-2,3-dioxo-2,3-dihydro-indol-1-yl)-propionic acid methyl ester (1.5 g) and tetrahydrofuran:water (1:1, 20 mL) was stirred at room temperature and treated with lithium hydroxide monohydrate (571 mg). The reaction mixture was then stirred for 2 h at room temperature. After this time, the reaction mixture was concentrated in vacuo to remove the tetrahydrofuran. The remaining aqueous layer was diluted with water and then acidified to pH=2 with a 1N aqueous hydrochloric acid solution. The aqueous layer was then extracted with ethyl acetate and the organic layers combined, dried over sodium sulfate, filtered and the filterate concentrated in vacuo to afford 3-cyclopentyl-2-(5-methoxy-2,3-dioxo-2,3-dihydro-indol-1-yl)-propionic acid (1.3 g, 90%).

A mixture of 3-cyclopentyl-2-(5-methoxy-2,3-dioxo-2,3-dihydro-indol-1-yl)-propionic acid (80 mg) in methylene chloride (5 mL) was cooled to 0° C. and treated with oxalyl chloride (26 μL) followed by N,N-dimethylformamide (one drop) and stirred for 30 min at 0° C. After this time the reaction mixture was concentrated in vacuo. The residue was dissolved in tetrahydrofuran (5 mL) and then treated with pyrazin-2-ylamine (29 mg) and 2,6-lutidine (88 μL). The resulting reaction mixture was then stirred at room temperature for 16 h. After this time, the reaction mixture was concentrated in vacuo and the residue dissolved in ethyl acetate (10 mL), and washed with a 0.5N aqueous hydrochloric acid solution (2×3 mL), saturated aqueous sodium bicarbonate solution (2×3 mL) and brine (4 mL). The organic layers were dried over sodium sulfate, filtered and the filterate concentrated in vacuo. The crude material was purified by Biotage column chromatography (silica gel, 15% ethyl acetate/hexanes) to afford 3-cyclopentyl-2-(5-methoxy-2,3-dioxo-2,3-dihydro-indol-1-yl)-N-pyrazin-2-yl-propionamide (33 mg, 33%): LCMS ESI calcd for C₂₁H₂₂N₄O₄(M+H)⁺ 395, observed 395.

### Example 46

### 3-Cyclopentyl-2-(5-methoxy-2,3-dioxo-2,3-dihydro-indol-1-yl)-N-pyridin-2-yl-propionamide

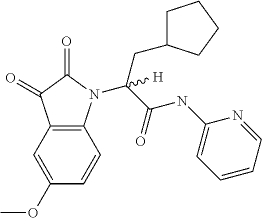

A mixture of 3-cyclopentyl-2-(5-methoxy-2,3-dioxo-2,3-dihydro-indol-1-yl)-propionic acid (prepared as in Example 45, 75 mg) and pyridin-2-ylamine (27 mg) in N,N-dimethylformamide (2 mL) at room temperature was treated with N,N-diisopropylethyl amine (68 μL) and (benzotriazol-1-yloxytris(dimethylamino)phosphonium hexafluorophosphate (157 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 3-cyclopentyl-2-(5-methoxy-2,3-dioxo-2,3-dihydro-indol-1-yl)-N-pyridin-2-yl-propionamide (30 mg, 32%) as a brown solid: LCMS ESI calcd for C₂₂H₂₃N₃O₄ (M+H)⁺ 394, observed 394.

### Example 47

### 3-Cyclopentyl-2-(5-methoxy-2,3-dioxo-2,3-dihydro-indol-1-yl)-N-(1-methyl-1H-pyrazol-3-yl)-propionamide

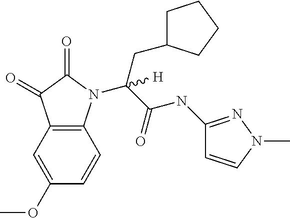

A mixture of 3-cyclopentyl-2-(5-methoxy-2,3-dioxo-2,3-dihydro-indol-1-yl)-propionic acid (prepared as in Example 45, 75 mg) and 1-methyl-1H-pyrazol-3-ylamine (28 mg) in N,N-dimethylformamide (2 mL) at room temperature was treated with N,N-diisopropylethyl amine (68 μL) and (benzotriazol-1-yloxytris(dimethylamino)phosphonium hexafluorophosphate (157 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 3-cyclopentyl-2-(5-methoxy-2,3-dioxo-2,3-dihydro-indol-1-yl)-N-(1-methyl-1H-pyrazol-3-yl)-propionamide (12 mg, 13%) as a brown solid: LCMS ESI calcd for C₂₁H₂₄N₄O₄ (M+H)⁺ 397, observed 397.

### Example 48

### 3-Cyclopentyl-2-(5-methoxy-2,3-dioxo-2,3-dihydro-indol-1-yl)-N-thiazol-2-yl-propionamide

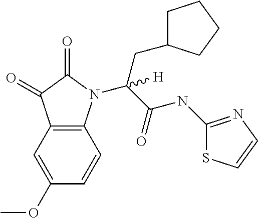

A mixture of 3-cyclopentyl-2-(5-methoxy-2,3-dioxo-2,3-dihydro-indol-1-yl)-propionic acid (prepared as in Example 45, 75 mg) and thiazol-2-ylamine (28 mg) in N,N-dimethylformamide (2 mL) at room temperature was treated with N,N-diisopropylethyl amine (68 μL) and (benzotriazol-1-yloxytris(dimethylamino)phosphonium hexafluorophosphate (157 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 3-cyclopentyl-2-(5-methoxy-2,3-dioxo-2,3-dihydro-indol-1-yl)-N-thiazol-2-yl-propionamide (80 mg, 85%) as a brown solid: LCMS ESI calcd for C₂₀H₂₁N₃O₄S (M+H)⁺ 400, observed 409.

### Example 49

### 3-Cyclopentyl-2-(5-methoxy-2-oxo-2,3-dihydro-indol-1-yl)-N-pyridin-2-yl-propionamide

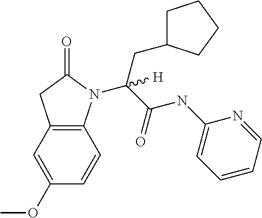

A mixture of 3-cyclopentyl-2-(5-methoxy-2,3-dioxo-2,3-dihydro-indol-1-yl)-propionic acid (prepared as in Example 45, 400 mg) and hydrazine hydrate (1 mL) were placed in a sealed tube and heated at 120° C. for 30 min. The reaction mixture was then cooled to room temperature and concentrated in vacuo. The residue was dissolved in methylene chloride (15 mL) and washed with a 0.5N aqueous hydrochloric acid solution (2×7 mL) and a saturated aqueous brine solution (5 mL). The organic layer was then dried over sodium sulfate, filtered and the filterate concentrated in vacuo to afford of 3-cyclopentyl-2-(5-methoxy-2-oxo-2,3-dihydro-indol-1-yl)-propionic acid (355 mg, 93%).

A mixture of 3-cyclopentyl-2-(5-methoxy-2-oxo-2,3-dihydro-indol-1-yl)-propionic acid (90 mg) and benzene (5 mL) was treated with thionyl chloride (26 μL) and heated at 80° C. for 2 h. After this time, the mixture was concentrated in vacuo to remove the benzene and the residue was dissolved in tetrahydrofuran (5 mL). The resulting solution was then treated with pyridin-2-ylamine (34 mg) and 2,6-lutidine (104 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(5-methoxy-2-oxo-2,3-dihydro-indol-1-yl)-N-pyridin-2-yl-propionamide (6 mg, 5%): LCMS ESI calcd for C₂₂H₂₅N₃O₃ (M+H)⁺ 380, observed 380.

### Example 50

### 3-Cyclopentyl-2-(5-methoxy-2-oxo-2,3-dihydro-indol-1-yl)-N-(1-methyl-1H-pyrazol-3-yl)-propionamide

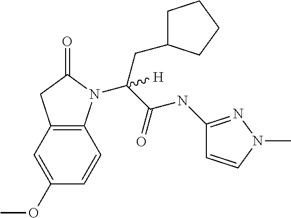

A mixture of 3-cyclopentyl-2-(5-methoxy-2-oxo-2,3-dihydro-indol-1-yl)-propionic acid (prepared as in Example 49, 50 mg) and benzene (3 mL) was treated with thionyl chloride (15 μL) and heated at 80° C. for 2 h. After this time, the mixture was concentrated in vacuo to remove the benzene and the residue was dissolved in tetrahydrofuran (3 mL). The resulting solution was then treated with 1-methyl-1H-pyrazol-3-ylamine (19 mg) and 2,6-lutidine (58 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(5-methoxy-2-oxo-2,3-dihydro-indol-1-yl)-N-(1-methyl-1H-pyrazol-3-yl)-propionamide (19 mg, 30%) as an off-white sticky solid: LCMS ESI calcd for C₂₁H₂₆N₄O₃ (M+H)⁺ 383, observed 383.

### Example 51

### 3-Cyclopentyl-2-(5-methoxy-2-oxo-2,3-dihydro-indol-1-yl)-N-thiazol-2-yl-propionamide

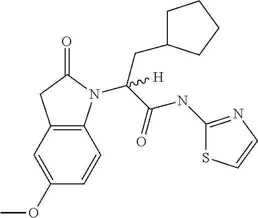

A mixture of 3-cyclopentyl-2-(5-methoxy-2-oxo-2,3-dihydro-indol-1-yl)-propionic acid (prepared as in Example 49, 50 mg) and benzene (3 mL) was treated with thionyl chloride (15 μL) and heated at 80° C. for 2 h. After this time, the mixture was concentrated in vacuo to remove the benzene and the residue was dissolved in tetrahydrofuran (3 mL). The resulting solution was then treated with thiazol-2-ylamine (20 mg) and 2,6-lutidine (58 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(5-methoxy-2-oxo-2,3-dihydro-indol-1-yl)-N-thiazol-2-yl-propionamide (17 mg, 26%) as an off-white sticky solid: LCMS ESI calcd for C₂₀H₂₃N₃O₃S (M+H)⁺ 386, observed 386.

### Example 52

### 3-Cyclopentyl-2-(5-methoxy-2-oxo-2,3-dihydro-indol-1-yl)-N-pyrazin-2-yl-propionamide

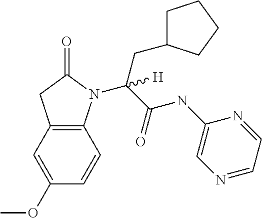

A mixture of 3-cyclopentyl-2-(5-methoxy-2-oxo-2,3-dihydro-indol-1-yl)-propionic acid (prepared as in Example 49, 50 mg) and benzene (3 mL) was treated with thionyl chloride (15 μL) and heated at 80° C. for 2 h. After this time, the mixture was concentrated in vacuo to remove the benzene and the residue was dissolved in tetrahydrofuran (3 mL). The resulting solution was then treated with pyrazin-2-ylamine (19 mg) and 2,6-lutidine (58 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(5-methoxy-2-oxo-2,3-dihydro-indol-1-yl)-N-pyrazin-2-yl-propionamide (19 mg, 30%) as an off-white sticky solid: LCMS ESI calcd for C₂₁H₂₄N₄O₃ (M+H)⁺ 381, observed 381.

### Example 53

### 2-(5-Methoxy-2,3-dioxo-2,3-dihydro-indol-1-yl)-4-methyl-pentanoic acid pyrazin-2-ylamide

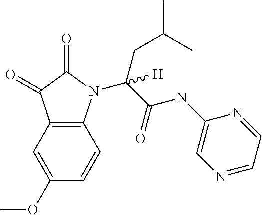

A stirred suspension of sodium hydride (60% dispersion in oil, 271 mg) in N,N-dimethylformamide (25 mL) at 0° C. was treated with a solution of 5-methoxy-1H-indole-2,3-dione (1.0 g) in N,N-dimethylformamide. The reaction mixture was stirred for 30 min at 0° C. and then 2-bromo-4-methyl-pentanoic acid methyl ester (prepared as in Example 9, 1.4 g) was added and it was stirred for 1 h at 0° C. It was then slowly allowed to warm to room temperature and stirred for another 3 h at room temperature. After this time, the reaction mixture was diluted with water and extracted with methylene chloride. The organic layers were combined and dried over sodium sulfate, filtered and the filterate concentrated in vacuo. The crude material was purified by column chromatography (silica gel) to afford 2-(5-methoxy-2,3-dioxo-2,3-dihydro-indol-1-yl)-4-methyl-pentanoic acid methyl ester (1.5 g, 87%).

A mixture of 2-(5-methoxy-2,3-dioxo-2,3-dihydro-indol-1-yl)-4-methyl-pentanoic acid methyl ester (1.5 g) and tetrahydrofuran:water (1:1, 20 mL) was stirred at room temperature and treated with lithium hydroxide monohydrate (620 mg). The reaction mixture was then stirred for 2 h at room temperature. After this time, the reaction mixture was concentrated in vacuo to remove the tetrahydrofuran. The remaining aqueous layer was diluted with water and then acidified to pH=2 with a 1N aqueous hydrochloric acid solution. The aqueous layer was then extracted with ethyl acetate and the organic layers combined, dried over sodium sulfate, filtered and the filterate concentrated in vacuo to afford 2-(5-methoxy-2,3-dioxo-2,3-dihydro-indol-1-yl)-4-methyl-pentanoic acid (1.3 g, 91%).

A mixture of 2-(5-methoxy-2,3-dioxo-2,3-dihydro-indol-1-yl)-4-methyl-pentanoic acid (80 mg) in methylene chloride (5 mL) was cooled to 0° C. and treated with oxalyl chloride (28 μL) followed by N,N-dimethylformamide (one drop) and stirred for 30 min at 0° C. After this time the reaction mixture was concentrated in vacuo. The residue was dissolved in tetrahydrofuran (5 mL) and then treated with pyrazin-2-ylamine (31 mg) and 2,6-lutidine (96 μL). The resulting reaction mixture was then stirred at room temperature for 16 h. After this time, the reaction mixture was concentrated in vacuo and the residue dissolved in ethyl acetate (10 mL), and washed with a 0.5N aqueous hydrochloric acid solution (2×3 mL), saturated aqueous sodium bicarbonate solution (2×3 mL) and brine (4 mL). The organic layers were dried over sodium sulfate, filtered and the filterate concentrated in vacuo. The crude material was purified by Biotage column chromatography (silica gel, 15% ethyl acetate/hexanes) to afford 2-(5-methoxy-2,3-dioxo-2,3-dihydro-indol-1-yl)-4-methyl-pentanoic acid pyrazin-2-ylamide (64 mg, 63%): LCMS ESI calcd for C₁₉H₂₀N₄O₄ (M+H)⁺ 369, observed 369.

### Example 54

### 2-(5-Methoxy-2,3-dioxo-2,3-dihydro-indol-1-yl)-4-methyl-pentanoic acid pyridin-2-ylamide

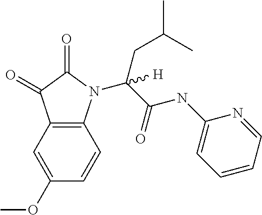

A mixture of 2-(5-methoxy-2,3-dioxo-2,3-dihydro-indol-1-yl)-4-methyl-pentanoic acid (prepared as in Example 53, 75 mg) and pyridin-2-ylamine (29 mg) in N,N-dimethylformamide (2 mL) at room temperature was treated with N,N-diisopropylethyl amine (70 μL) and (benzotriazol-1-yloxytris(dimethylamino)phosphonium hexafluorophosphate (171 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(5-methoxy-2,3-dioxo-2,3-dihydro-indol-1-yl)-4-methyl-pentanoic acid pyridin-2-ylamide (40 mg, 42%) as a brown solid: LCMS ESI calcd for C₂₀H₂₁N₃O₄ (M+H)⁺ 368, observed 368.

### Example 55

### 2-(5-Methoxy-2,3-dioxo-2,3-dihydro-indol-1-yl)-4-methyl-pentanoic acid thiazol-2-ylamide

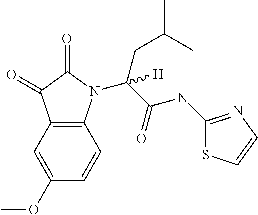

A mixture of 2-(5-methoxy-2,3-dioxo-2,3-dihydro-indol-1-yl)-4-methyl-pentanoic acid (prepared as in Example 53, 75 mg) and thiazol-2-ylamine (31 mg) in N,N-dimethylformamide (2 mL) at room temperature was treated with N,N-diisopropylethyl amine (70 μL) and (benzotriazol-1-yloxytris(dimethylamino)phosphonium hexafluorophosphate (171 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(5-methoxy-2,3-dioxo-2,3-dihydro-indol-1-yl)-4-methyl-pentanoic acid thiazol-2-ylamide (70 mg, 73%) as a brown solid: LCMS ESI calcd for C₁₈H₁₉N₃O₄S (M+H)⁺ 374, observed 374.

### Example 56

### 2-(5-Methoxy-2-oxo-2,3-dihydro-indol-1-yl)-4-methyl-pentanoic acid thiazol-2-ylamide

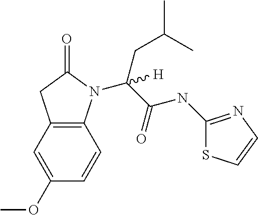

A mixture of 2-(5-methoxy-2,3-dioxo-2,3-dihydro-indol-1-yl)-4-methyl-pentanoic acid (prepared as in Example 53, 400 mg) and hydrazine hydrate (1 mL) were placed in a sealed tube and heated at 120° C. for 30 min. The reaction mixture was then cooled to room temperature and concentrated in vacuo. The residue was dissolved in methylene chloride (15 mL) and washed with a 0.5N aqueous hydrochloric acid solution (2×7 mL) and a saturated aqueous brine solution (5 mL). The organic layer was then dried over sodium sulfate, filtered and the filterate concentrated in vacuo to afford 2-(5-methoxy-2-oxo-2,3-dihydro-indol-1-yl)-4-methyl-pentanoic acid (360 mg, 95%).

A mixture of 2-(5-methoxy-2-oxo-2,3-dihydro-indol-1-yl)-4-methyl-pentanoic acid (50 mg) and benzene (3 mL) was treated with thionyl chloride (15 μL) and heated at 80° C. for 2 h. After this time, the mixture was concentrated in vacuo to remove the benzene and the residue was dissolved in tetrahydrofuran (3 mL). The resulting solution was then treated with thiazol-2-ylamine (20 mg) and 2,6-lutidine (58 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(5-methoxy-2-oxo-2,3-dihydro-indol-1-yl)-4-methyl-pentanoic acid thiazol-2-ylamide (20 mg, 31%): LCMS ESI calcd for C₁₈H₂₁N₃O₃S (M+H)⁺ 360, observed 360.

### Example 57

### 2-(5-Methoxy-2-oxo-2,3-dihydro-indol-1-yl)-4-methyl-pentanoic acid pyridin-2-ylamide

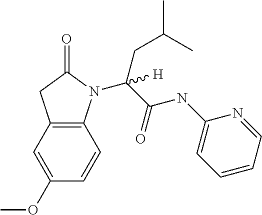

A mixture of 2-(5-methoxy-2-oxo-2,3-dihydro-indol-1-yl)-4-methyl-pentanoic (prepared as in Example 56, 90 mg) and benzene (5 mL) was treated with thionyl chloride (28 μL) and heated at 80° C. for 2 h. After this time, the mixture was concentrated in vacuo to remove the benzene and the residue was dissolved in tetrahydrofuran (5 mL). The resulting solution was then treated with pyridin-2-ylamine (37 mg) and 2,6-lutidine (114 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(5-methoxy-2-oxo-2,3-dihydro-indol-1-yl)-4-methyl-pentanoic acid pyridin-2-ylamide (7 mg, 6%): LCMS ESI calcd for C₂₀H₂₃N₃O₃ (M+H)⁺ 354, observed 354.

### Example 58

### 2-(5-Methoxy-2-oxo-2,3-dihydro-indol-1-yl)-4-methyl-pentanoic acid pyrazin-2-ylamide

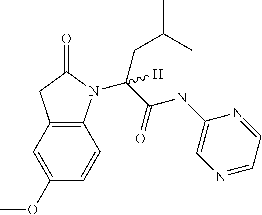

A mixture of 2-(5-methoxy-2-oxo-2,3-dihydro-indol-1-yl)-4-methyl-pentanoic (prepared as in Example 56, 50 mg) and benzene (3 mL) was treated with thionyl chloride (15 μL) and heated at 80° C. for 2 h. After this time, the mixture was concentrated in vacuo to remove the benzene and the residue was dissolved in tetrahydrofuran (3 mL). The resulting solution was then treated with pyrazin-2-ylamine (19 mg, 0.20) and 2,6-lutidine (58 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(5-methoxy-2-oxo-2,3-dihydro-indol-1-yl)-4-methyl-pentanoic acid pyrazin-2-ylamide (15 mg, 34%) as an off-white sticky solid: LCMS ESI calcd for C₁₉H₂₂N₄O₃ (M+H)⁺ 355, observed 355.

### Example 59

### 3-Cyclopentyl-N-[1-(2-hydroxy-2-methyl-propyl)-1H-pyrazol-3-yl]-2-(2-oxo-2,3-dihydro-indol-1-yl)-propionamide

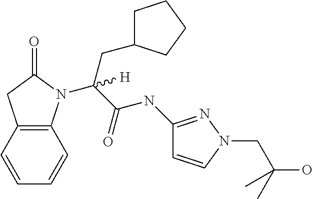

A mixture of 3-cyclopentyl-2-(2-oxo-2,3-dihydro-indol-1-yl)-propionic acid (prepared as in Example 2, 70 mg), (benzotriazol-1-yloxytris(dimethylamino)phosphonium hexafluorophosphate (147 mg) and 1-(3-amino-pyrazol-1-yl)-2-methyl-propan-2-ol (prepared as in US20080021032 Example 80, 48 mg) in methylene chloride (10 mL) at room temperature was treated with N,N-diisopropylethyl amine (133 μL) and stirred overnight for 16 h at room temperature. After this time, the reaction mixture was diluted with water (10 mL) and extracted with ethyl acetate (3×15 mL). The organic layers were then combined and washed with a 1N aqueous hydrochloric acid solution (10 mL), a saturated aqueous sodium bicarbonate solution (10 mL) and then dried over magnesium sulfate, filtered and the filterate concentrated in vacuo. The crude material was then purified using an Analogix Intelliflash system (4 g silica gel column, 50% ethyl acetate/hexanes to 100% ethyl acetate) to afford 3-cyclopentyl-N-[1-(2-hydroxy-2-methyl-propyl)-1H-pyrazol-3-yl]-2-(2-oxo-2,3-dihydro-indol-1-yl)-propionamide (41 mg, 39%) of a light orange foam: LRMS ES calcd for C₂₃H₃₀N₄O₃ (M+H)⁺ 411, observed 411

### Example 60

### 2-(5-Bromo-2,3-dioxo-2,3-dihydro-indol-1-yl)-3-cyclopentyl-N-pyrazin-2-yl-propionamide

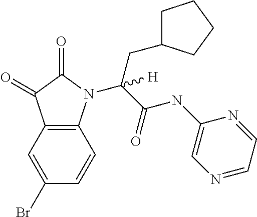

A stirred suspension of sodium hydride (60% dispersion in oil, 212 mg) in N,N-dimethylformamide (10 mL) at 0° C. was treated with a solution of 5-bromo-1H-indole-2,3-dione (1.0 g) in N,N-dimethylformamide. The reaction mixture was stirred for 30 min at 0° C. and then 2-bromo-3-cyclopentyl-propionic acid methyl ester (prepared as in Example 1, 1.25 g) was added and it was stirred for 1 h at 0° C. It was then slowly allowed to warm to room temperature and stirred for another 3 h at room temperature. After this time, the reaction mixture was diluted with water and extracted with chloroform. The organic layers were combined and dried over sodium sulfate, filtered and the filterate concentrated in vacuo. The crude material was purified by column chromatography (silica gel, 5% ethyl acetate/hexanes) to afford 2-(5-bromo-2,3-dioxo-2,3-dihydro-indol-1-yl)-3-cyclopentyl-propionic acid methyl ester (720 mg, 43%).

A mixture of 2-(5-bromo-2,3-dioxo-2,3-dihydro-indol-1-yl)-3-cyclopentyl-propionic acid methyl ester (715 mg) and tetrahydrofuran:water (1:1, 20 mL) was stirred at room temperature and treated with lithium hydroxide monohydrate (158 mg). The reaction mixture was then stirred at room temperature. The reaction mixture was concentrated in vacuo to remove the tetrahydrofuran. The remaining aqueous layer was diluted with water and then acidified to pH=2 with a dilute aqueous hydrochloric acid solution. The aqueous layer was then extracted with ethyl acetate and the organic layers combined, dried over sodium sulfate, filtered and the filterate concentrated in vacuo to afford 2-(5-bromo-2,3-dioxo-2,3-dihydro-indol-1-yl)-3-cyclopentyl-propionic acid (700 mg, 99%).

A mixture of 2-(5-bromo-2,3-dioxo-2,3-dihydro-indol-1-yl)-3-cyclopentyl-propionic acid (70 mg) in methylene chloride (3 mL) was cooled to 0° C. and treated with oxalyl chloride (20 μL) followed by N,N-dimethylformamide (one drop) and stirred for 30 min at 0° C. After this time the reaction mixture was concentrated in vacuo. The residue was dissolved in tetrahydrofuran (3 mL) and then treated with pyrazin-2-ylamine (22 mg) and 2,6-lutidine (67 μL). The resulting reaction mixture was then stirred at room temperature overnight. After this time, the reaction mixture was diluted with ethyl acetate and washed with a 1N aqueous hydrochloric acid solution, a saturated aqueous sodium bicarbonate solution and brine. The organic layers were dried over sodium sulfate, filtered and the filterate concentrated in vacuo. The crude material was purified by column chromatography (silica gel) to afford 2-(5-bromo-2,3-dioxo-2,3-dihydro-indol-1-yl)-3-cyclopentyl-N-pyrazin-2-yl-propionamide (82 mg, 97%): LCMS ESI calcd for C₂₀H₁₉BrN₄O₃ (M+H)⁺ 443, observed 443.

### Example 61

### 2-(5-Bromo-2,3-dioxo-2,3-dihydro-indol-1-yl)-3-cyclopentyl-N-pyridin-2-yl-propionamide

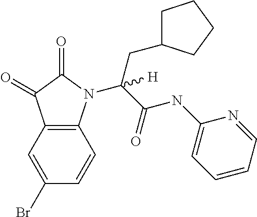

A mixture of 2-(5-bromo-2,3-dioxo-2,3-dihydro-indol-1-yl)-3-cyclopentyl-propionic acid (prepared as in Example 60, 70 mg) and pyridin-2-ylamine (22 mg) in N,N-dimethylformamide (5 mL) at room temperature was treated with N,N-diisopropylethyl amine (100 μL) and (benzotriazol-1-yloxytris(dimethylamino)phosphonium hexafluorophosphate (127 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel, 5% ethyl acetate/hexanes) to afford 2-(5-bromo-2,3-dioxo-2,3-dihydro-indol-1-yl)-3-cyclopentyl-N-pyridin-2-yl-propionamide (18 mg, 21%) as a yellow solid LCMS ESI calcd for C₂₁H₂₀BrN₃O₃ (M+H)⁺ 442, observed 442.

### Example 62

### 2-(5-Bromo-2,3-dioxo-2,3-dihydro-indol-1-yl)-3-cyclopentyl-N-(1-methyl-1H-pyrazol-3-yl)-propionamide

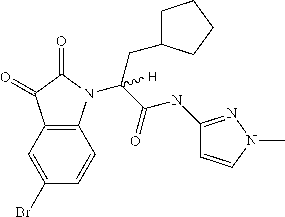

A mixture of 2-(5-bromo-2,3-dioxo-2,3-dihydro-indol-1-yl)-3-cyclopentyl-propionic acid (prepared as in Example 60, 70 mg) and 1-methyl-1H-pyrazol-3-ylamine (22 mg) in N,N-dimethylformamide (5 mL) at room temperature was treated with N,N-diisopropylethyl amine (100 μL) and (benzotriazol-1-yloxytris(dimethylamino)phosphonium hexafluorophosphate (127 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel, 5% ethyl acetate/hexanes) to afford 2-(5-bromo-2,3-dioxo-2,3-dihydro-indol-1-yl)-3-cyclopentyl-N-(1-methyl-1H-pyrazol-3-yl)-propionamide (41 mg, 48%) as a yellow solid: LCMS ESI calcd for C₂₀H₂₁BrN₄O₃S (M+H)⁺ 445, observed 445.

### Example 63

### 2-(5-Bromo-2,3-dioxo-2,3-dihydro-indol-1-yl)-3-cyclopentyl-N-thiazol-2-yl-propionamide

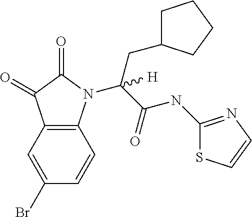

A mixture of 2-(5-bromo-2,3-dioxo-2,3-dihydro-indol-1-yl)-3-cyclopentyl-propionic acid (prepared as in Example 60, 70 mg) and thiazol-2-ylamine (23 mg) in N,N-dimethylformamide (5 mL) at room temperature was treated with N,N-diisopropylethyl amine (100 μL) and (benzotriazol-1-yloxytris(dimethylamino)phosphonium hexafluorophosphate (127 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel, 5% ethyl acetate/hexanes) to afford 2-(5-bromo-2,3-dioxo-2,3-dihydro-indol-1-yl)-3-cyclopentyl-N-thiazol-2-yl-propionamide (32 mg, 37%) as a yellow solid: LCMS ESI calcd for C₁₉H₁₈BrN₃O₃S (M+H)⁺ 448, observed 448.

### Example 64

### 2-(5-Bromo-2-oxo-2,3-dihydro-indol-1-yl)-3-cyclopentyl-N-pyridin-2-yl-propionamide

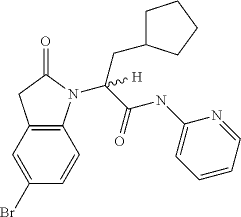

A mixture of 2-(5-bromo-2,3-dioxo-2,3-dihydro-indol-1-yl)-3-cyclopentyl-propionic acid (prepared as in Example 60, 400 mg) and hydrazine hydrate (2 mL) were placed in a sealed tube and heated at 120° C. for 25 min. The reaction mixture was then cooled to room temperature and concentrated in vacuo. The residue was dissolved in methylene chloride and washed with a 0.5N aqueous hydrochloric acid solution. The organic layer was then dried over sodium sulfate, filtered and the filterate concentrated in vacuo and the reaction was purified using Biotage column chromatography (silica gel) to afford 2-(5-bromo-2-oxo-2,3-dihydro-indol-1-yl)-3-cyclopentyl-propionic acid (310 mg, 81%).

A mixture of 2-(5-bromo-2-oxo-2,3-dihydro-indol-1-yl)-3-cyclopentyl-propionic acid (60 mg) and benzene (3 mL) was treated with thionyl chloride (17 μL) and heated at 60° C. for 1 h. After this time, the mixture was concentrated in vacuo to remove the benzene and the residue was dissolved in tetrahydrofuran (3 mL). The resulting solution was then treated with pyridin-2-ylamine (19 mg) and 2,6-lutidine (60 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 2-(5-bromo-2-oxo-2,3-dihydro-indol-1-yl)-3-cyclopentyl-N-pyridin-2-yl-propionamide (17 mg, 23%) as a grey solid: LCMS ESI calcd for C₂₁H₂₂BrN₃O₂ (M+H)⁺ 428, observed 428.

### Example 65

### 2-(5-Bromo-2-oxo-2,3-dihydro-indol-1-yl)-3-cyclopentyl-N-(1-methyl-1H-pyrazol-3-yl)-propionamide

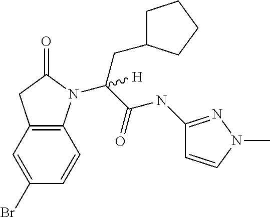

A mixture of 2-(5-bromo-2-oxo-2,3-dihydro-indol-1-yl)-3-cyclopentyl-propionic acid (prepared as in Example 64, 60 mg) and benzene (3 mL) was treated with thionyl chloride (17 μL) and heated at 60° C. for 1 h. After this time, the mixture was concentrated in vacuo to remove the benzene and the residue was dissolved in tetrahydrofuran (3 mL). The resulting solution was then treated with 1-methyl-1H-pyrazol-3-ylamine (20 mg) and 2,6-lutidine (60 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 2-(5-bromo-2-oxo-2,3-dihydro-indol-1-yl)-3-cyclopentyl-N-(1-methyl-1H-pyrazol-3-yl)-propionamide (37 mg, 50%) as a brown oil: LCMS ESI calcd for C₂₀H₂₃BrN₄O₂ (M+H)⁺ 431, observed 431.

### Example 66

### 2-(5-Bromo-2-oxo-2,3-dihydro-indol-1-yl)-3-cyclopentyl-N-thiazol-2-yl-propionamide

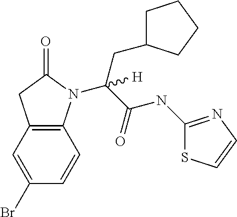

A mixture of 2-(5-bromo-2-oxo-2,3-dihydro-indol-1-yl)-3-cyclopentyl-propionic acid (prepared as in Example 64, 60 mg) and benzene (3 mL) was treated with thionyl chloride (17 μL) and heated at 60° C. for 1 h. After this time, the mixture was concentrated in vacuo to remove the benzene and the residue was dissolved in tetrahydrofuran (3 mL). The resulting solution was then treated with thiazol-2-ylamine (20 mg) and 2,6-lutidine (60 μL,) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 2-(5-bromo-2-oxo-2,3-dihydro-indol-1-yl)-3-cyclopentyl-N-pyridin-2-yl-propionamide (72 mg, 100%): LCMS ESI calcd for C₁₉H₂₀BrN₃O₂S (M+H)⁺ 434, observed 434.

### Example 67

### 2-(5-Bromo-2-oxo-2,3-dihydro-indol-1-yl)-3-cyclopentyl-N-pyrazin-2-yl-propionamide

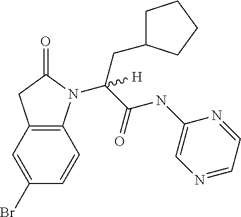

A mixture of 2-(5-bromo-2-oxo-2,3-dihydro-indol-1-yl)-3-cyclopentyl-propionic acid (prepared as in Example 64, 60 mg) and benzene (3 mL) was treated with thionyl chloride (17 μL) and heated at 60° C. for 1 h. After this time, the mixture was concentrated in vacuo to remove the benzene and the residue was dissolved in tetrahydrofuran (3 mL). The resulting solution was then treated with pyridin-2-ylamine (19 mg) and 2,6-lutidine (60 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 2-(5-bromo-2-oxo-2,3-dihydro-indol-1-yl)-3-cyclopentyl-N-pyrazin-2-yl-propionamide (20 mg, 27%) as a brown solid: LCMS ESI calcd for C₂₀H₂₁BrN₄O₂ (M+H)⁺ 429, observed 429.

### Example 68

### 2-(5-Bromo-2,3-dioxo-2,3-dihydro-indol-1-yl)-4-methyl-pentanoic acid pyrazin-2-ylamide

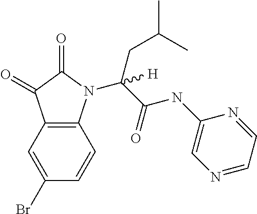

A stirred suspension of sodium hydride (60% dispersion in oil, 212 mg) in N,N-dimethylformamide (10 mL) at 0° C. was treated with a solution of 5-bromo-1H-indole-2,3-dione (1.0 g) in N,N-dimethylformamide. The reaction mixture was stirred for 30 min at 0° C. and then 2-bromo-4-methyl-pentanoic acid methyl ester (prepared as in Example 9, 1.11 g) was added and it was stirred for 1 h at 0° C. It was then slowly allowed to warm to room temperature and stirred for another 3 h at room temperature. After this time, the reaction mixture was diluted with water and extracted with chloroform. The organic layers were combined and dried over sodium sulfate, filtered and the filterate concentrated in vacuo. The crude material was purified by column chromatography (silica gel, 5% ethyl acetate/hexanes) to afford 2-(5-bromo-2,3-dioxo-2,3-dihydro-indol-1-yl)-4-methyl-pentanoic acid methyl ester (1.4 g, 90%).

A mixture of 2-(5-bromo-2,3-dioxo-2,3-dihydro-indol-1-yl)-4-methyl-pentanoic acid methyl ester (1.4 g) and tetrahydrofuran:water (1:1, 20 mL) was stirred at room temperature and treated with lithium hydroxide monohydrate (331 mg). The reaction mixture was then stirred at room temperature. The reaction mixture was concentrated in vacuo to remove the tetrahydrofuran. The remaining aqueous layer was diluted with water and then acidified to pH=2 with a dilute aqueous hydrochloric acid solution. The aqueous layer was then extracted with ethyl acetate and the organic layers combined, dried over sodium sulfate, filtered and the filterate concentrated in vacuo to afford 2-(5-bromo-2,3-dioxo-2,3-dihydro-indol-1-yl)-4-methyl-pentanoic acid (1.3 g, 97%).

A mixture of 2-(5-bromo-2,3-dioxo-2,3-dihydro-indol-1-yl)-4-methyl-pentanoic acid (100 mg) in methylene chloride (5 mL) was cooled to 0° C. and treated with oxalyl chloride (30 μL) followed by N,N-dimethylformamide (one drop) and stirred for 30 min at 0° C. After this time the reaction mixture was concentrated in vacuo. The residue was dissolved in tetrahydrofuran (5 mL) and then treated with pyrazin-2-ylamine (34 mg) and 2,6-lutidine (102 μL). The resulting reaction mixture was then stirred at room temperature overnight. After this time, the reaction mixture was diluted with ethyl acetate and washed with a 1N aqueous hydrochloric acid solution, a saturated aqueous sodium bicarbonate solution and brine. The organic layers were dried over sodium sulfate, filtered and the filterate concentrated in vacuo. The crude material was purified by column chromatography (silica gel) to afford 2-(5-bromo-2,3-dioxo-2,3-dihydro-indol-1-yl)-4-methyl-pentanoic acid pyrazin-2-ylamide (78 mg, 63%) as an orange solid: LCMS ESI calcd for C₁₈H₁₇BrN₄O₃ (M+H)⁺ 417, observed 417.

### Example 69

### 2-(5-Bromo-2,3-dioxo-2,3-dihydro-indol-1-yl)-4-methyl-pentanoic acid pyridin-2-ylamide

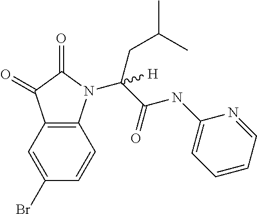

A mixture of 2-(5-bromo-2,3-dioxo-2,3-dihydro-indol-1-yl)-4-methyl-pentanoic acid (prepared as in Example 68, 130 mg) and pyridin-2-ylamine (43 mg) in N,N-dimethylformamide (5 mL) at room temperature was treated with N,N-diisopropylethyl amine (200 μL) and (benzotriazol-1-yloxytris(dimethylamino)phosphonium hexafluorophosphate (253 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel, 5% ethyl acetate/hexanes) to afford 2-(5-bromo-2,3-dioxo-2,3-dihydro-indol-1-yl)-4-methyl-pentanoic acid pyridin-2-ylamide (100 mg, 63%) as a yellow solid: LCMS ESI calcd for C₁₉H₁₈BrN₃O₃ (M+H)⁺ 416, observed 416.

### Example 70

### 2-(5-Bromo-2,3-dioxo-2,3-dihydro-indol-1-yl)-4-methyl-pentanoic acid (1-methyl-1H-pyrazol-3-yl)-amide

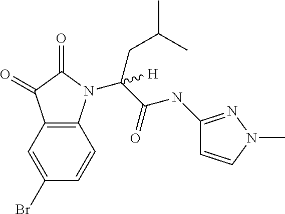

A mixture of 2-(5-bromo-2,3-dioxo-2,3-dihydro-indol-1-yl)-4-methyl-pentanoic acid (prepared as in Example 68, 100 mg) and 1-methyl-1H-pyrazol-3-ylamine (35 mg) in N,N-dimethylformamide (5 mL) at room temperature was treated with N,N-diisopropylethyl amine (160 μL) and (benzotriazol-1-yloxytris(dimethylamino)phosphonium hexafluorophosphate (195 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel, 5% ethyl acetate/hexanes) to afford 2-(5-bromo-2,3-dioxo-2,3-dihydro-indol-1-yl)-4-methyl-pentanoic acid (1-methyl-1H-pyrazol-3-yl)-amide (60 mg, 49%) as an orange solid: LCMS ESI calcd for C₁₈H₁₉BrN₄O₃ (M+H)⁺ 419, observed 419.

### Example 71

### 2-(5-Bromo-2,3-dioxo-2,3-dihydro-indol-1-yl)-4-methyl-pentanoic acid thiazol-2-ylamide

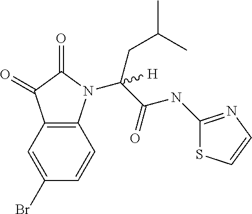

A mixture of 2-(5-bromo-2,3-dioxo-2,3-dihydro-indol-1-yl)-4-methyl-pentanoic acid (prepared as in Example 68, 100 mg) and thiazol-2-ylamine (36 mg) in N,N-dimethylformamide (5 mL) at room temperature was treated with N,N-diisopropylethyl amine (160 μL) and (benzotriazol-1-yloxytris(dimethylamino)phosphonium hexafluorophosphate (195 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel, 5% ethyl acetate/hexanes) to afford 2-(5-bromo-2,3-dioxo-2,3-dihydro-indol-1-yl)-4-methyl-pentanoic acid thiazol-2-ylamide (30 mg, 24%) as an orange solid: LCMS ESI calcd for C₁₇H₁₆BrN₃O₃S (M+H)⁺ 422, observed 422.

### Example 72

### 2-(5-Bromo-2-oxo-2,3-dihydro-indol-1-yl)-4-methyl-pentanoic acid thiazol-2-ylamide

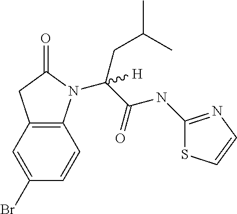

A mixture of 2-(5-bromo-2,3-dioxo-2,3-dihydro-indol-1-yl)-4-methyl-pentanoic acid (prepared as in Example 68, 600 mg) and hydrazine hydrate (1 mL) were placed in a sealed tube and heated at 120° C. for 25 min. The reaction mixture was then cooled to room temperature and concentrated in vacuo. The residue was dissolved in methylene chloride and washed with a 0.5N aqueous hydrochloric acid solution. The organic layer was then dried over sodium sulfate, filtered and the filterate concentrated in vacuo to afford 2-(5-bromo-2-oxo-2,3-dihydro-indol-1-yl)-4-methyl-pentanoic acid (530 mg, 92%).

A mixture of 2-(5-bromo-2-oxo-2,3-dihydro-indol-1-yl)-4-methyl-pentanoic acid (70 mg) and benzene (3 mL) was treated with thionyl chloride (19 μL) and heated at 60° C. for 1 h. After this time, the mixture was concentrated in vacuo to remove the benzene and the residue was dissolved in tetrahydrofuran (3 mL). The resulting solution was then treated with thiazol-2-ylamine (26 mg) and 2,6-lutidine (75 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 2-(5-bromo-2-oxo-2,3-dihydro-indol-1-yl)-4-methyl-pentanoic acid thiazol-2-ylamide (60 mg, 69%) as a white solid: LCMS ESI calcd for C₁₇H₁₈BrN₃O₂S (M+H)⁺ 408, observed 408.

### Example 73

### 2-(5-Bromo-2-oxo-2,3-dihydro-indol-1-yl)-4-methyl-pentanoic acid (1-methyl-1H-pyrazol-3-yl)-amide

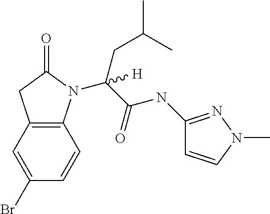

A mixture of 2-(5-bromo-2-oxo-2,3-dihydro-indol-1-yl)-4-methyl-pentanoic acid (prepared as in Example 72, 100 mg) and 1-methyl-1H-pyrazol-3-ylamine (36 mg) in N,N-dimethylformamide (5 mL) at room temperature was treated with N,N-diisopropylethyl amine (160 μL) and (benzotriazol-1-yloxytris(dimethylamino)phosphonium hexafluorophosphate (203 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(5-bromo-2-oxo-2,3-dihydro-indol-1-yl)-4-methyl-pentanoic acid (1-methyl-1H-pyrazol-3-yl)-amide (45 mg, 36%) as a white solid: LCMS ESI calcd for C₁₈H₂₁BrN₄O₂ (M+H)⁺ 405, observed 405.

### Example 74

### 2-(5-Bromo-2-oxo-2,3-dihydro-indol-1-yl)-4-methyl-pentanoic acid pyridin-2-ylamide

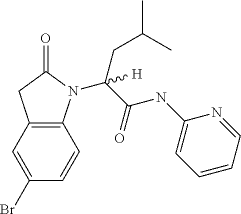

A mixture of 2-(5-bromo-2-oxo-2,3-dihydro-indol-1-yl)-4-methyl-pentanoic acid (prepared as in Example 72, 100 mg) and benzene (5 mL) was treated with thionyl chloride (27 μL) and heated at 80° C. for 2 h. After this time, the mixture was concentrated in vacuo to remove the benzene and the residue was dissolved in tetrahydrofuran (5 mL). The resulting solution was then treated with pyridin-2-ylamine (35 mg) and 2,6-lutidine (107 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(5-bromo-2-oxo-2,3-dihydro-indol-1-yl)-4-methyl-pentanoic acid pyridin-2-ylamide (6 mg, 5%): LCMS ESI calcd for C₁₉H₂₀BrN₃O₂ (M+H)⁺ 402, observed 402.

### Example 75

### 2-(5-Bromo-2-oxo-2,3-dihydro-indol-1-yl)-4-methyl-pentanoic acid pyrazin-2-ylamide

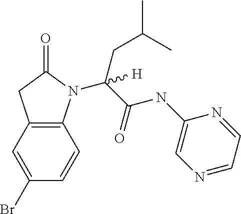

A mixture of 2-(5-bromo-2-oxo-2,3-dihydro-indol-1-yl)-4-methyl-pentanoic acid (prepared as in Example 72, 100 mg) and benzene (5 mL) was treated with thionyl chloride (27 μL) and heated at 60° C. for 1 h. After this time, the mixture was concentrated in vacuo to remove the benzene and the residue was dissolved in tetrahydrofuran (5 mL). The resulting solution was then treated with pyrazin-2-ylamine (35 mg) and 2,6-lutidine (107 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by prep thin layer chromatography to afford 2-(5-bromo-2-oxo-2,3-dihydro-indol-1-yl)-4-methyl-pentanoic acid pyrazin-2-ylamide (16 mg, 13%) as an off white solid: LCMS ESI calcd for C₁₈H₁₉BrN₄O₂ (M+H)⁺ 403, observed 403.

### Example 76

### 3-Cyclopentyl-2-(5-methyl-2,3-dioxo-2,3-dihydro-indol-1-yl)-N-pyrazin-2-yl-propionamide

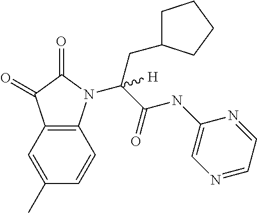

A stirred suspension of sodium hydride (60% dispersion in oil, 300 mg) in N,N-dimethylformamide (15 mL) at 0° C. was treated with a solution of 5-methyl-1H-indole-2,3-dione (1.0 g) in N,N-dimethylformamide. The reaction mixture was stirred for 30 min at 0° C. and then 2-bromo-3-cyclopentyl-propionic acid methyl ester (prepared as in Example 1, 1.75 g) was added and it was stirred for 1 h at 0° C. It was then slowly allowed to warm to room temperature and stirred for another 3 h at room temperature. After this time, the reaction mixture was diluted with water and extracted with chloroform. The organic layers were combined and dried over sodium sulfate, filtered and the filterate concentrated in vacuo. The crude material was purified by column chromatography (silica gel, 5% ethyl acetate/hexanes) to afford 3-cyclopentyl-2-(5-methyl-2,3-dioxo-2,3-dihydro-indol-1-yl)-propionic acid methyl ester (1.3 g, 67%).

A mixture of 3-cyclopentyl-2-(5-methyl-2,3-dioxo-2,3-dihydro-indol-1-yl)-propionic acid methyl ester (1.3 g) and tetrahydrofuran:water (1:1, 24 mL) was stirred at room temperature and treated with lithium hydroxide monohydrate (344 mg). The reaction mixture was then stirred at room temperature. The reaction mixture was concentrated in vacuo to remove the tetrahydrofuran. The remaining aqueous layer was diluted with water and then acidified to pH=2 with a dilute aqueous hydrochloric acid solution. The aqueous layer was then extracted with ethyl acetate and the organic layers combined, dried over sodium sulfate, filtered and the filterate concentrated in vacuo to afford 3-cyclopentyl-2-(5-methyl-2,3-dioxo-2,3-dihydro-indol-1-yl)-propionic acid (1.15 g, 76%).

A mixture of 3-cyclopentyl-2-(5-methyl-2,3-dioxo-2,3-dihydro-indol-1-yl)-propionic acid (70 mg) in methylene chloride (3 mL) was cooled to 0° C. and treated with oxalyl chloride (24 μL) followed by N,N-dimethylformamide (one drop) and stirred for 30 min at 0° C. After this time the reaction mixture was concentrated in vacuo. The residue was dissolved in tetrahydrofuran (3 mL) and then treated with pyrazin-2-ylamine (27 mg) and 2,6-lutidine (81 μL). The resulting reaction mixture was then stirred at room temperature overnight. After this time, the reaction mixture was diluted with ethyl acetate and washed with a 1N aqueous hydrochloric acid solution, a saturated aqueous sodium bicarbonate solution and brine. The organic layers were dried over sodium sulfate, filtered and the filterate concentrated in vacuo. The crude material was purified by column chromatography (silica gel) to afford 3-cyclopentyl-2-(5-methyl-2,3-dioxo-2,3-dihydro-indol-1-yl)-N-pyrazin-2-yl-propionamide (32 mg, 36%): LCMS ESI calcd for C₂₁H₂₁N₄O₃ (M+H)⁺ 379, observed 379.

### Example 77

### 3-Cyclopentyl-2-(5-methyl-2,3-dioxo-2,3-dihydro-indol-1-yl)-N-pyridin-2-yl-propionamide

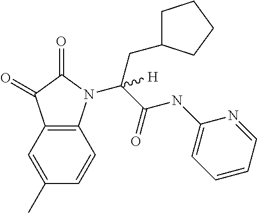

A mixture of 3-cyclopentyl-2-(5-methyl-2,3-dioxo-2,3-dihydro-indol-1-yl)-propionic acid (prepared as in Example 76, 70 mg) and pyridin-2-ylamine (27 mg) in N,N-dimethylformamide (5 mL) at room temperature was treated with N,N-diisopropylethyl amine (122 μL) and (benzotriazol-1-yloxytris(dimethylamino)phosphonium hexafluorophosphate (155 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel, 5% ethyl acetate/hexanes) to afford 3-cyclopentyl-2-(5-methyl-2,3-dioxo-2,3-dihydro-indol-1-yl)-N-pyridin-2-yl-propionamide (8 mg, 9%) as a yellow solid: LCMS ESI calcd for C₂₂H₂₃N₃O₃ (M+H)⁺ 378, observed 378.

### Example 78

### 3-Cyclopentyl-2-(5-methyl-2,3-dioxo-2,3-dihydro-indol-1-yl)-N-(1-methyl-1H-pyrazol-3-yl)-propionamide

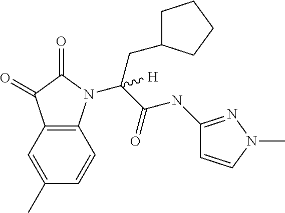

A mixture of 3-cyclopentyl-2-(5-methyl-2,3-dioxo-2,3-dihydro-indol-1-yl)-propionic acid (prepared as in Example 76, 70 mg) and 1-methyl-1H-pyrazol-3-ylamine (27 mg) in N,N-dimethylformamide (5 mL) at room temperature was treated with N,N-diisopropylethyl amine (122 μL) and (benzotriazol-1-yloxytris(dimethylamino)phosphonium hexafluorophosphate (155 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel, 5% ethyl acetate/hexanes) to afford 3-cyclopentyl-2-(5-methyl-2,3-dioxo-2,3-dihydro-indol-1-yl)-N-(1-methyl-1H-pyrazol-3-yl)-propionamide (65 mg, 73%) as a yellow solid: LCMS ESI calcd for C₂₁H₂₄N₄O₃ (M+H)⁺ 381, observed 381.

### Example 79

### 3-Cyclopentyl-2-(5-methyl-2,3-dioxo-2,3-dihydro-indol-1-yl)-N-thiazol-2-yl-propionamide

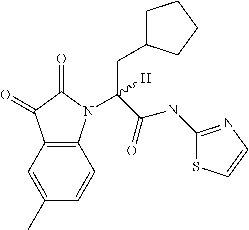

A mixture of 3-cyclopentyl-2-(5-methyl-2,3-dioxo-2,3-dihydro-indol-1-yl)-propionic acid (prepared as in Example 76, 70 mg) and thiazol-2-ylamine (28 mg) in N,N-dimethylformamide (5 mL) at room temperature was treated with N,N-diisopropylethyl amine (122 μL) and (benzotriazol-1-yloxytris(dimethylamino)phosphonium hexafluorophosphate (155 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel, 5% ethyl acetate/hexanes) to afford 3-cyclopentyl-2-(5-methyl-2,3-dioxo-2,3-dihydro-indol-1-yl)-N-thiazol-2-yl-propionamide (75 mg, 54%) as a yellow solid: LCMS ESI calcd for C₂₀H₂₁N₃O₃S (M+H)⁺ 384, observed 384.

### Example 80

### 3-Cyclopentyl-2-(5-methyl-2-oxo-2,3-dihydro-indol-1-yl)-N-pyridin-2-yl-propionamide

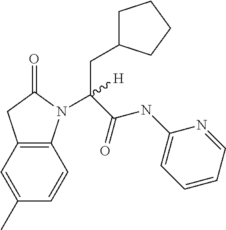

A mixture of 3-cyclopentyl-2-(5-methyl-2,3-dioxo-2,3-dihydro-indol-1-yl)-propionic acid (prepared as in Example 76, 520 mg) and hydrazine hydrate (1 mL) were placed in a sealed tube and heated at 120° C. for 15 min. The reaction mixture was then cooled to room temperature and concentrated in vacuo. The residue was dissolved in methylene chloride (30 mL) and washed with a 0.5N aqueous hydrochloric acid solution (2×10 mL) and brine (10 mL). The organic layer was then dried over sodium sulfate, filtered and the filterate concentrated in vacuo to afford 3-cyclopentyl-2-(5-methyl-2-oxo-2,3-dihydro-indol-1-yl)-propionic acid (480 mg, 97%).

A mixture of 3-cyclopentyl-2-(5-methyl-2-oxo-2,3-dihydro-indol-1-yl)-propionic acid (60 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with pyridin-2-ylamine (24 mg) and 2,6-lutidine (73 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(5-methyl-2-oxo-2,3-dihydro-indol-1-yl)-N-pyridin-2-yl-propionamide (10 mg, 13%) as a brown liquid: LCMS ESI calcd for C₂₂H₂₅N₃O₂ (M+H)⁺ 364, observed 364.

### Example 81

### 3-Cyclopentyl-2-(5-methyl-2-oxo-2,3-dihydro-indol-1-yl)-N-(1-methyl-1H-pyrazol-3-yl)-propionamide

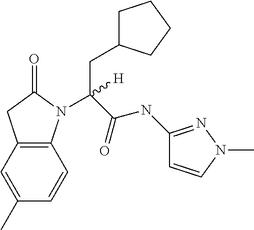

A mixture of 3-cyclopentyl-2-(5-methyl-2-oxo-2,3-dihydro-indol-1-yl)-propionic acid (prepared as in Example 80, 60 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with 1-methyl-1H-pyrazol-3-ylamine (24 mg) and 2,6-lutidine (73 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(5-methyl-2-oxo-2,3-dihydro-indol-1-yl)-N-(1-methyl-1H-pyrazol-3-yl)-propionamide (10 mg, 13%) as a red liquid: LCMS ESI calcd for C₂₁H₂₆N₄O₂ (M+H)⁺ 367, observed 367.

### Example 82

### 3-Cyclopentyl-2-(5-methyl-2-oxo-2,3-dihydro-indol-1-yl)-N-thiazol-2-yl-propionamide

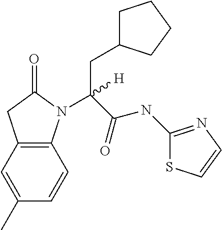

A mixture of 3-cyclopentyl-2-(5-methyl-2-oxo-2,3-dihydro-indol-1-yl)-propionic acid (prepared as in Example 80, 60 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with thiazol-2-ylamine (25 mg) and 2,6-lutidine (73 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(5-methyl-2-oxo-2,3-dihydro-indol-1-yl)-N-thiazol-2-yl-propionamide (53 mg, 69%) as an off-white solid: LCMS ESI calcd for C₂₀H₂₃N₃O₂S (M+H)⁺ 370, observed 370.

### Example 83

### 3-Cyclopentyl-2-(5-methyl-2-oxo-2,3-dihydro-indol-1-yl)-N-pyrazin-2-yl-propionamide

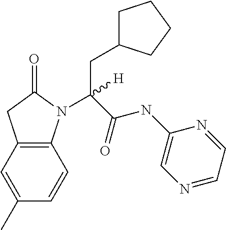

A mixture of 3-cyclopentyl-2-(5-methyl-2-oxo-2,3-dihydro-indol-1-yl)-propionic acid (prepared as in Example 80, 60 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with pyrazin-2-ylamine (24 mg) and 2,6-lutidine (73 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(5-methyl-2-oxo-2,3-dihydro-indol-1-yl)-N-pyrazin-2-yl-propionamide (20 mg, 25%) as a yellow solid: LCMS ESI calcd for C₂₁H₂₄N₄O₂ (M+H)⁺ 365, observed 365.

### Example 84

### 4-Methyl-2-(5-methyl-2,3-dioxo-2,3-dihydro-indol-1-yl)-pentanoic acid pyrazin-2-ylamide

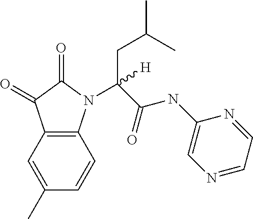

A stirred suspension of sodium hydride (60% dispersion in oil, 297 mg) in N,N-dimethylformamide (15 mL) at 0° C. was treated with a solution of 5-methyl-1H-indole-2,3-dione (1.0 g) in N,N-dimethylformamide. The reaction mixture was stirred for 30 min at 0° C. and then 2-bromo-4-methyl-pentanoic acid methyl ester (prepared as in Example 9, 1.56 g) was added and it was stirred for 1 h at 0° C. It was then slowly allowed to warm to room temperature and stirred for another 3 h at room temperature. After this time, the reaction mixture was diluted with water and extracted with chloroform. The organic layers were combined and dried over sodium sulfate, filtered and the filterate concentrated in vacuo. The crude material was purified by column chromatography (silica gel, 5% ethyl acetate/hexanes) to afford 4-methyl-2-(5-methyl-2,3-dioxo-2,3-dihydro-indol-1-yl)-pentanoic acid methyl ester (1.0 g, 56%).

A mixture of 4-methyl-2-(5-methyl-2,3-dioxo-2,3-dihydro-indol-1-yl)-pentanoic acid methyl ester (1.0 g) and tetrahydrofuran:water (1:1, 20 mL) was stirred at room temperature and treated with lithium hydroxide monohydrate (290 mg). The reaction mixture was then stirred at room temperature. The reaction mixture was concentrated in vacuo to remove the tetrahydrofuran. The remaining aqueous layer was diluted with water and then acidified to pH=2 with a dilute aqueous hydrochloric acid solution. The aqueous layer was then extracted with ethyl acetate and the organic layers combined, dried over sodium sulfate, filtered and the filterate concentrated in vacuo to afford 4-methyl-2-(5-methyl-2,3-dioxo-2,3-dihydro-indol-1-yl)-pentanoic acid (950 mg, 99%).

A mixture of 4-methyl-2-(5-methyl-2,3-dioxo-2,3-dihydro-indol-1-yl)-pentanoic acid (100 mg) in methylene chloride (5 mL) was cooled to 0° C. and treated with oxalyl chloride (37 μL) followed by N,N-dimethylformamide (one drop) and stirred for 30 min at 0° C. After this time the reaction mixture was concentrated in vacuo. The residue was dissolved in tetrahydrofuran (5 mL) and then treated with pyrazin-2-ylamine (42 mg) and 2,6-lutidine (127 μL). The resulting reaction mixture was then stirred at room temperature overnight. After this time, the reaction mixture was diluted with ethyl acetate and washed with a 1N aqueous hydrochloric acid solution, a saturated aqueous sodium bicarbonate solution and brine. The organic layers were dried over sodium sulfate, filtered and the filterate concentrated in vacuo. The crude material was purified by column chromatography (silica gel) to afford 4-methyl-2-(5-methyl-2,3-dioxo-2,3-dihydro-indol-1-yl)-pentanoic acid pyrazin-2-ylamide (70 mg, 55%) as an orange solid: LCMS ESI calcd for C₁₉H₂₀N₄O₃ (M+H)⁺ 353, observed 353.

### Example 85

### 4-Methyl-2-(5-methyl-2,3-dioxo-2,3-dihydro-indol-1-yl)-pentanoic acid pyridin-2-ylamide

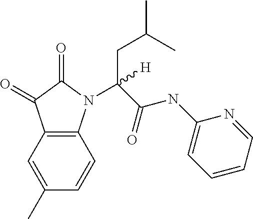

A mixture of 4-methyl-2-(5-methyl-2,3-dioxo-2,3-dihydro-indol-1-yl)-pentanoic acid (prepared as in Example 84, 100 mg) and pyridin-2-ylamine (42 mg) in N,N-dimethylformamide (5 mL) at room temperature was treated with N,N-diisopropylethyl amine (190 μL) and (benzotriazol-1-yloxytris(dimethylamino)phosphonium hexafluorophosphate (241 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel, 5% ethyl acetate/hexanes) to afford 4-methyl-2-(5-methyl-2,3-dioxo-2,3-dihydro-indol-1-yl)-pentanoic acid pyridin-2-ylamide (20 mg, 16%) as an orange solid: LCMS ESI calcd for C₂₀H₂₁N₃O₃ (M+H)⁺ 352, observed 352.

### Example 86

### 4-Methyl-2-(5-methyl-2,3-dioxo-2,3-dihydro-indol-1-yl)-pentanoic acid (1-methyl-1H-pyrazol-3-yl)-amide

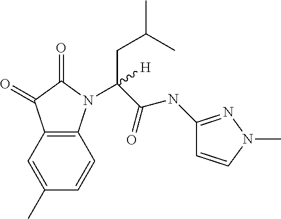

A mixture of 4-methyl-2-(5-methyl-2,3-dioxo-2,3-dihydro-indol-1-yl)-pentanoic acid (prepared as in Example 84, 100 mg) and 1-methyl-1H-pyrazol-3-ylamine (43 mg) in N,N-dimethylformamide (5 mL) at room temperature was treated with N,N-diisopropylethyl amine (190 μL) and (benzotriazol-1-yloxytris(dimethylamino)phosphonium hexafluorophosphate (241 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel, 5% ethyl acetate/hexanes) to afford 4-methyl-2-(5-methyl-2,3-dioxo-2,3-dihydro-indol-1-yl)-pentanoic acid (1-methyl-1H-pyrazol-3-yl)-amide (95 mg, 74%) as an orange solid: LCMS ESI calcd for C₁₉H₂₂N₄O₃ (M+H)⁺ 355, observed 355.

### Example 87

### 4-Methyl-2-(5-methyl-2,3-dioxo-2,3-dihydro-indol-1-yl)-pentanoic acid thiazol-2-ylamide

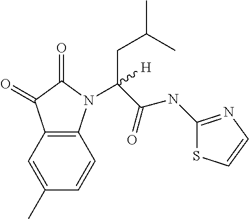

A mixture of 4-methyl-2-(5-methyl-2,3-dioxo-2,3-dihydro-indol-1-yl)-pentanoic acid (prepared as in Example 84, 100 mg) and thiazol-2-ylamine (44 mg) in N,N-dimethylformamide (5 mL) at room temperature was treated with N,N-diisopropylethyl amine (190 μL) and (benzotriazol-1-yloxytris(dimethylamino)phosphonium hexafluorophosphate (241 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel, 5% ethyl acetate/hexanes) to afford 4-methyl-2-(5-methyl-2,3-dioxo-2,3-dihydro-indol-1-yl)-pentanoic acid thiazol-2-ylamide (80 mg, 62%) as a red solid: LCMS ESI calcd for C₁₈H₁₉N₃O₃S (M+H)⁺ 358, observed 358.

### Example 88

### 4-Methyl-2-(5-methyl-2-oxo-2,3-dihydro-indol-1-yl)-pentanoic acid thiazol-2-ylamide

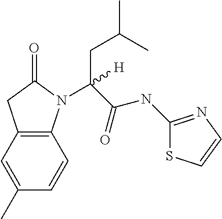

A mixture of 4-methyl-2-(5-methyl-2,3-dioxo-2,3-dihydro-indol-1-yl)-pentanoic acid (prepared as in Example 84, 600 mg) and hydrazine hydrate (1 mL) were placed in a sealed tube and heated at 120° C. for 25 min. The reaction mixture was then cooled to room temperature and concentrated in vacuo. The residue was dissolved in methylene chloride and washed with a 0.5N aqueous hydrochloric acid solution. The organic layer was then dried over sodium sulfate, filtered and the filterate concentrated in vacuo to afford 4-methyl-2-(5-methyl-2-oxo-2,3-dihydro-indol-1-yl)-pentanoic acid (500 mg, 88%).

A mixture of 4-methyl-2-(5-methyl-2-oxo-2,3-dihydro-indol-1-yl)-pentanoic acid (60 mg) and benzene (3 mL) was treated with thionyl chloride (20 μL) and heated at 60° C. for 1 h. After this time, the mixture was concentrated in vacuo to remove the benzene and the residue was dissolved in tetrahydrofuran (3 mL). The resulting solution was then treated with thiazol-2-ylamine (28 mg) and 2,6-lutidine (80 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 4-methyl-2-(5-methyl-2-oxo-2,3-dihydro-indol-1-yl)-pentanoic acid thiazol-2-ylamide (20 mg, 79%) as a pale orange solid: LCMS ESI calcd for C₁₈H₂₁N₃O₂S (M+H)⁺ 344, observed 344.

### Example 89

### 4-Methyl-2-(5-methyl-2-oxo-2,3-dihydro-indol-1-yl)-pentanoic acid (1-methyl-1H-pyrazol-3-yl)-amide

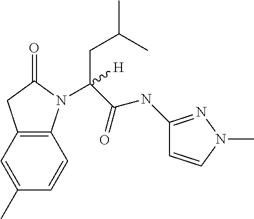

A mixture of 4-methyl-2-(5-methyl-2-oxo-2,3-dihydro-indol-1-yl)-pentanoic acid (prepared as in Example 88, 60 mg) and benzene (3 mL) was treated with thionyl chloride (20 μL) and heated at 60° C. for 1 h. After this time, the mixture was concentrated in vacuo to remove the benzene and the residue was dissolved in tetrahydrofuran (3 mL). The resulting solution was then treated with 1-methyl-1H-pyrazol-3-ylamine (27 mg) and 2,6-lutidine (80 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 4-methyl-2-(5-methyl-2-oxo-2,3-dihydro-indol-1-yl)-pentanoic acid (1-methyl-1H-pyrazol-3-yl)-amide (19 mg, 24%) as an orange solid: LCMS ESI calcd for C₁₉H₂₄N₄O₂ (M+H)⁺ 341, observed 341.

### Example 90

### 4-Methyl-2-(5-methyl-2-oxo-2,3-dihydro-indol-1-yl)-pentanoic acid pyridin-2-ylamide

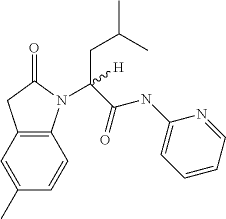

A mixture of 4-methyl-2-(5-methyl-2-oxo-2,3-dihydro-indol-1-yl)-pentanoic acid (prepared as in Example 88, 60 mg) and benzene (3 mL) was treated with thionyl chloride (20 μL) and heated at 60° C. for 1 h. After this time, the mixture was concentrated in vacuo to remove the benzene and the residue was dissolved in tetrahydrofuran (3 mL). The resulting solution was then treated with pyridin-2-ylamine (26 mg) and 2,6-lutidine (80 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 4-methyl-2-(5-methyl-2-oxo-2,3-dihydro-indol-1-yl)-pentanoic acid pyridin-2-ylamide (10 mg, 13%) as a pale orange solid: LCMS ESI calcd for C₂₀H₂₃N₃O₂ (M+H)⁺ 338, observed 338.

### Example 91

### 4-Methyl-2-(5-methyl-2-oxo-2,3-dihydro-indol-1-yl)-pentanoic acid pyrazin-2-ylamide

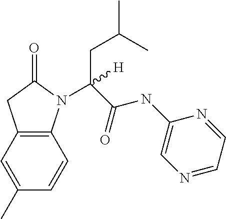

A mixture of 4-methyl-2-(5-methyl-2-oxo-2,3-dihydro-indol-1-yl)-pentanoic acid (prepared as in Example 88, 60 mg) and benzene (3 mL) was treated with thionyl chloride (20 μL) and heated at 60° C. for 1 h. After this time, the mixture was concentrated in vacuo to remove the benzene and the residue was dissolved in tetrahydrofuran (3 mL). The resulting solution was then treated with pyrazin-2-ylamine (26 mg) and 2,6-lutidine (80 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 4-methyl-2-(5-methyl-2-oxo-2,3-dihydro-indol-1-yl)-pentanoic acid pyrazin-2-ylamide (14 mg, 18%) as an orange solid: LCMS ESI calcd for C₁₉H₂₂N₄O₂ (M+H)⁺ 339, observed 339.

### Example 92

### 2-(5-Chloro-2,3-dioxo-2,3-dihydro-indol-1-yl)-3-cyclopentyl-N-thiazol-2-yl-propionamide

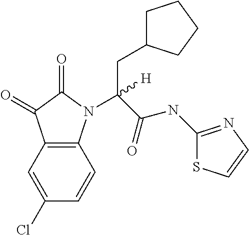

A mixture of sodium hydride (60% dispersion in oil, 268 mg) in N,N-dimethylformamide (5 mL) was cooled to 0° C. in an ice bath and then slowly treated with a solution of 5-chloroisatin (1.00 g) in N,N-dimethylformamide (5 mL). The resulting dark blue reaction mixture was stirred at 0° C. for 30 min. After this period of time, the reaction was treated with 2-bromo-3-cyclopentyl-propionic acid methyl ester (prepared as in Example 1, 1.55 g) and then stirred at 0° C. for 1 h and then warmed to room temperature and stirred for another 3 h. After this time, the reaction mixture was diluted with water (30 mL) and extracted with chloroform (3×30 mL) and the organics were then washed with a saturated aqueous brine solution (30 mL), dried over sodium sulfate, filtered to remove the drying agent and then the filterate concentrated in vacuo with silica gel (4 g). The silica gel with absorbed material was then placed into a SIM cartridge and purified by flash chromatography (Analogix SF25-40 g column, 25% ethyl acetate/hexanes) to afford 2-(5-chloro-2,3-dioxo-2,3-dihydro-indol-1-yl)-3-cyclopentyl-propionic acid methyl ester (1.28 g, 69%) as a bright orange oil.

A mixture of 2-(5-chloro-2,3-dioxo-2,3-dihydro-indol-1-yl)-3-cyclopentyl-propionic acid methyl ester (1.28 g) and lithium hydroxide monohydrate (381 mg) in tetrahydrofuran:water (1:1, 30 mL) was stirred at room temperature for 2 h. After this time, the reaction mixture was concentrated in vacuo to remove the tetrahydrofuran and then diluted with an additional amount of water (15 mL) and then a 1 N aqueous hydrochloric acid solution was added until the pH=2. This aqueous layer was then extracted with ethyl acetate (2×30 mL) and the combined organic layers were dried over sodium sulfate, filtered to remove the drying agent and the filterate concentrated in vacuo to afford 2-(5-chloro-2,3-dioxo-2,3-dihydro-indol-1-yl)-3-cyclopentyl-propionic acid (1.12 g, 91%) as an orange solid.

A mixture of 2-(5-chloro-2,3-dioxo-2,3-dihydro-indol-1-yl)-3-cyclopentyl-propionic acid (100 mg) and 2-aminothiazole (38 mg) in N,N-dimethylformamide (2 mL) was stirred at room temperature and treated with diisopropylethyl amine (160 μL) and (benzotriazol-1-yloxytris(dimethylamino)phosphonium hexafluorophosphate (207 mg). The reaction mixture was then stirred at room temperature overnight. The reaction mixture was then diluted with ethyl acetate (20 mL) and washed with a saturated aqueous ammonium chloride solution (15 mL), a saturated aqueous sodium bicarbonate solution (15 mL), and a saturated aqueous brine solution (15 mL). The organic layer was then dried over sodium sulfate, filtered to remove the drying agent and the filterate concentrated in vacuo with silica gel (2 g).). The silica gel with absorbed material was then placed into a SIM cartridge and purified by flash chromatography (Analogix SF15-12 g column, 25% ethyl acetate/hexanes) to afford 2-(5-chloro-2,3-dioxo-2,3-dihydro-indol-1-yl)-3-cyclopentyl-N-thiazol-2-yl-propionamide (108 mg, 86%) as a bright orange foam: LRMS ES calcd for C₁₉H₁₈ClN₃O₃S (M+H)⁺ 404, observed 404.

### Example 93

### 2-(5-Chloro-2,3-dioxo-2,3-dihydro-indol-1-yl)-3-cyclopentyl-N-[1-((R)-2,3-dihydroxy-propyl)-1H-pyrazol-3-yl]-propionamide

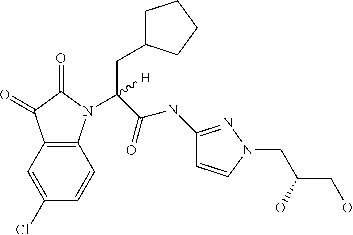

A mixture of 2-(5-chloro-2,3-dioxo-2,3-dihydro-indol-1-yl)-3-cyclopentyl-propionic acid (prepared as in Example 92, 100 mg) and 1-((R)-2,2-dimethyl-[1,3]dioxolan-4-ylmethyl)-1H-pyrazol-3-ylamine (prepared as in US2008/0021032, Example 35, 74 mg) in N,N-dimethylformamide (2 mL) was stirred at room temperature and treated with diisopropylethyl amine (160 μL) and (benzotriazol-1-yloxytris(dimethylamino)phosphonium hexafluorophosphate (207 mg). The reaction mixture was then stirred at room temperature overnight. The reaction mixture was then diluted with ethyl acetate (20 mL) and washed with a saturated aqueous ammonium chloride solution (15 mL), a saturated aqueous sodium bicarbonate solution (15 mL), and a saturated aqueous brine solution (15 mL). The organic layer was then dried voer sodium sulfate, filtered to remove the drying agent and the filterate concentrated in vacuo with silica gel (2 g). The silica gel with absorbed material was then placed into a SIM cartridge and purified by flash chromatography (Analogix SF15-12 g column, 25%-40% ethyl acetate/hexanes) to afford 2-(5-chloro-2,3-dioxo-2,3-dihydro-indol-1-yl)-3-cyclopentyl-N-[1-((R)-2,2-dimethyl-[1,3]dioxolan-4-ylmethyl)-1H-pyrazol-3-yl]-propionamide (116 mg, 75%) as a bright orange foam.

A solution of 2-(5-chloro-2,3-dioxo-2,3-dihydro-indol-1-yl)-3-cyclopentyl-N-[1-((R)-2,2-dimethyl-[1,3]dioxolan-4-ylmethyl)-1H-pyrazol-3-yl]-propionamide (110 mg) in tetrahydrofuran (5 mL) was treated with a 2 N aqueous hydrochloric acid solution (5 mL) and stirred at room temperature for 2 h. After this time, the reaction mixture was diluted with ethyl acetate (25 mL) and washed with water (20 mL), a saturated aqueous sodium bicarbonate solution (20 mL), and a saturated aqueous brine solution (20 mL). The organic layers were dried over sodium sulfate, filtered to remove the drying agent and then concentrated in vacuo to afford 2-(5-chloro-2,3-dioxo-2,3-dihydro-indol-1-yl)-3-cyclopentyl-N-[14R)-2,3-dihydroxy-propyl)-1H-pyrazol-3-yl]-propionamide (103 mg, quant.) as a bright orange foam: LRMS ES calcd for C₂₂H₂₅ClN₄O₅ (M+H)⁺ 461, observed 461.

### Example 94

### 2-(5-Chloro-2,3-dioxo-2,3-dihydro-indol-1-yl)-3-cyclopentyl-N-pyridin-2-yl-propionamide

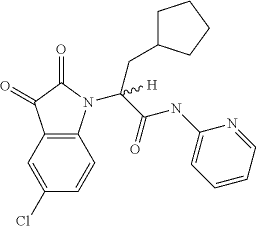

A mixture of 2-(5-chloro-2,3-dioxo-2,3-dihydro-indol-1-yl)-3-cyclopentyl-propionic acid (prepared as in Example 92, 50 mg) and pyridin-2-ylamine (20 mg) in N,N-dimethylformamide (1 mL) was stirred at room temperature and treated with diisopropylethyl amine (80 μL) and (benzotriazol-1-yloxytris(dimethylamino)phosphonium hexafluorophosphate (104 mg). The reaction mixture was then stirred at room temperature overnight. The reaction mixture was then diluted with ethyl acetate (20 mL) and washed with a saturated aqueous ammonium chloride solution (15 mL), a saturated aqueous sodium bicarbonate solution (15 mL), and a saturated aqueous brine solution (15 mL). The organic layer was then dried over sodium sulfate, filtered to remove the drying agent and the filterate concentrated in vacuo with silica gel (2 g). The silica gel with absorbed material was then placed into a SIM cartridge and purified by flash chromatography (Analogix SF10-8g column, 25% ethyl acetate/hexanes) to afford 2-(5-chloro-2,3-dioxo-2,3-dihydro-indol-1-yl)-3-cyclopentyl-N-pyridin-2-yl-propionamide (57 mg, 93%) as a bright orange foam: LRMS ES calcd for C₂₁H₂₀ClN₃O₃ (M+H)⁺ 398, observed 398.

### Example 95

### 2-(5-Chloro-2,3-dioxo-2,3-dihydro-indol-1-yl)-3-cyclopentyl-N-(1-methyl-1H-pyrazol-3-yl)-propionamide

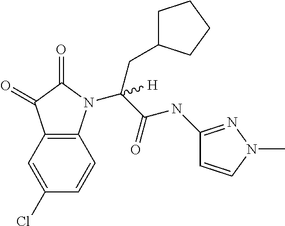

A mixture of 2-(5-chloro-2,3-dioxo-2,3-dihydro-indol-1-yl)-3-cyclopentyl-propionic acid (prepared as in Example 92, 100 mg) and 1-methyl-1H-pyrazol-3-ylamine (36 mg) in N,N-dimethylformamide (2 mL) was treated with diisopropylethyl amine (100 μL) and (benzotriazol-1-yloxytris(dimethylamino)phosphonium hexafluorophosphate (207 mg). The reaction mixture was then stirred at room temperature overnight and then diluted with ethyl acetate and the organic layer washed with a saturated aqueous ammonium chloride solution, saturated aqueous sodium bicarbonate solution, and a saturated aqueous brine solution. The organic layer was then dried over sodium sulfate, filtered to remove the drying agent and then the filterate was concentrated in vacuo and the residue was purified by silica gel column chromatography to afford 2-(5-chloro-2,3-dioxo-2,3-dihydro-indol-1-yl)-3-cyclopentyl-N-(1-methyl-1H-pyrazol-3-yl)-propionamide (60 mg, 48%) as an orange solid: LCMS ESI calcd for C₂₀H₂₁ClN₄O₃ (M+H)⁺ 401, observed 401.

### Example 96

### 2-(5-Chloro-2,3-dioxo-2,3-dihydro-indol-1-yl)-3-cyclopentyl-N-pyrazin-2-yl-propionamide

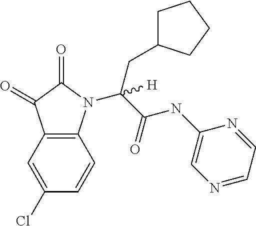

A mixture of 2-(5-chloro-2,3-dioxo-2,3-dihydro-indol-1-yl)-3-cyclopentyl-propionic acid (prepared as in Example 92, 60 mg) in methylene chloride (5 mL) was cooled to 0° C. and treated with oxalyl chloride (19 μL) followed by N,N-dimethylformamide (one drop) and stirred for 30 min at 0° C. After this time the reaction mixture was concentrated in vacuo. The residue was dissolved in tetrahydrofuran (5 mL) and then treated with pyrazin-2-ylamine (21 mg) and 2,6-lutidine (65 μL). The resulting reaction mixture was then stirred at room temperature for 16 h. After this time, the reaction mixture was concentrated in vacuo and the residue dissolved in ethyl acetate (10 mL), and washed with a 0.5N aqueous hydrochloric acid solution (2×3 mL), saturated aqueous sodium bicarbonate solution (2×3 mL) and brine (4 mL). The organic layers were dried over sodium sulfate, filtered and the filterate concentrated in vacuo. The crude material was purified by Biotage column chromatography (silica gel, 15% ethyl acetate/hexanes) to afford 2-(5-chloro-2,3-dioxo-2,3-dihydro-indol-1-yl)-3-cyclopentyl-N-pyrazin-2-yl-propionamide (22 mg, 30%): LCMS ESI calcd for C₂₀H₁₉ClN₄O₃ (M+H)⁺ 399, observed 399.

### Example 97

### 2-(5-Chloro-2-oxo-2,3-dihydro-indol-1-yl)-3-cyclopentyl-N-pyrazin-2-yl-propionamide

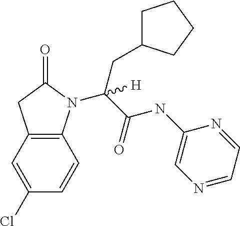

A mixture of 2-(5-chloro-2,3-dioxo-2,3-dihydro-indol-1-yl)-3-cyclopentyl-propionic acid (prepared as in Example 92, 450 mg) and hydrazine hydrate (2 mL) were placed in a sealed tube and heated at 120° C. for 25 min. The reaction mixture was then cooled to room temperature and concentrated in vacuo. The residue was dissolved in methylene chloride and washed with a 0.5N aqueous hydrochloric acid solution. The organic layer was then dried over sodium sulfate, filtered and the filterate concentrated in vacuo to afford 2-(5-chloro-2-oxo-2,3-dihydro-indol-1-yl)-3-cyclopentyl-propionic acid (290 mg, 67%).

A mixture of 2-(5-chloro-2-oxo-2,3-dihydro-indol-1-yl)-3-cyclopentyl-propionic acid (60 mg) and benzene (3 mL) were treated with thionyl chloride (46 μL) and heated at 60° C. for 1 h. After this time, the mixture was concentrated in vacuo to remove the benzene and the residue was dissolved in tetrahydrofuran (3 mL). The resulting solution was then treated with pyrazin-2-ylamine (22 mg) and 2,6-lutidine (58 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 2-(5-chloro-2-oxo-2,3-dihydro-indol-1-yl)-3-cyclopentyl-N-pyrazin-2-yl-propionamide (8 mg, 11%) as an off white solid: LCMS ESI calcd for C₂₀H₂₁ClN₄O₂ (M+H)⁺ 385, observed 385.

### Example 98

### 2-(5-Chloro-2-oxo-2,3-dihydro-indol-1-yl)-3-cyclopentyl-N-thiazol-2-yl-propionamide

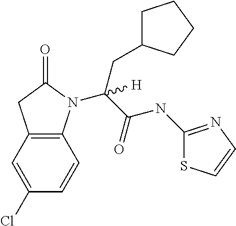

A mixture of 2-(5-chloro-2-oxo-2,3-dihydro-indol-1-yl)-3-cyclopentyl-propionic acid (prepared as in Example 97, 60 mg) and benzene (3 mL) were treated with thionyl chloride (46 μL) and heated at 60° C. for 1 h. After this time, the mixture was concentrated in vacuo to remove the benzene and the residue was dissolved in tetrahydrofuran (3 mL). The resulting solution was then treated with thiazol-2-ylamine (24 mg) and 2,6-lutidine (58 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 2-(5-chloro-2-oxo-2,3-dihydro-indol-1-yl)-3-cyclopentyl-N-thiazol-2-yl-propionamide (15 mg, 20%) as an off white solid: LCMS ESI calcd for C₁₉H₂₀ClN₃O₂S (M+H)⁺ 390, observed 390.

### Example 99

### 2-(5-Chloro-2-oxo-2,3-dihydro-indol-1-yl)-3-cyclopentyl-N-(1-methyl-1H-pyrazol-3-yl)-propionamide

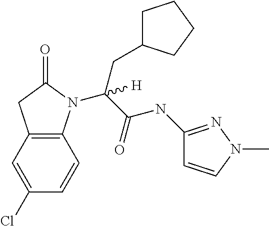

A mixture of 2-(5-chloro-2-oxo-2,3-dihydro-indol-1-yl)-3-cyclopentyl-propionic acid (prepared as in Example 97, 85 mg) and benzene (3 mL) were treated with thionyl chloride (65 μL) and heated at 60° C. for 1 h. After this time, the mixture was concentrated in vacuo to remove the benzene and the residue was dissolved in tetrahydrofuran (3 mL). The resulting solution was then treated with 1-methyl-1H-pyrazol-3-ylamine (32 mg) and 2,6-lutidine (82 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 2-(5-chloro-2-oxo-2,3-dihydro-indol-1-yl)-3-cyclopentyl-N-(1-methyl-1H-pyrazol-3-yl)-propionamide (15 mg, 14%) as an orange solid: LCMS ESI calcd for C₂₀H₂₃ClN₄O₂ (M+H)⁺ 387, observed 387.

### Example 100

### 2-(5-Chloro-2-oxo-2,3-dihydro-indol-1-yl)-3-cyclopentyl-N-pyridin-2-yl-propionamide

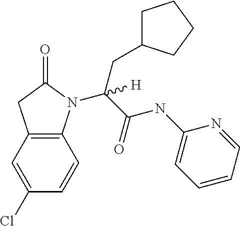

A mixture of 2-(5-chloro-2-oxo-2,3-dihydro-indol-1-yl)-3-cyclopentyl-propionic acid (prepared as in Example 97, 85 mg) and benzene (3 mL) were treated with thionyl chloride (65 μL) and heated at 60° C. for 1 h. After this time, the mixture was concentrated in vacuo to remove the benzene and the residue was dissolved in tetrahydrofuran (3 mL). The resulting solution was then treated with pyridin-2-ylamine (32 mg) and 2,6-lutidine (82 μL) and stirred at room temperature for 16 h. The reaction mixture was then concentrated in vacuo to remove the tetrahydrofuran and the residue was dissolved in ethyl acetate and washed with a 0.5N aqueous hydrochloric acid solution and a saturated aqueous sodium bicarbonate solution. The organic layer was then dried over sodium sulfate, filtered and the filterate concentrated in vacuo and the residue was purified by column chromatography (silica gel) to afford 2-(5-chloro-2-oxo-2,3-dihydro-indol-1-yl)-3-cyclopentyl-N-pyridin-2-yl-propionamide (15 mg, 14%) as an orange solid: LCMS ESI calcd for C₂₁H₂₂ClN₃O₂ (M+H)⁺ 384, observed 384.

### Example 101

### 2-(5-Chloro-2,3-dioxo-2,3-dihydro-indol-1-yl)-4-methyl-pentanoic acid thiazol-2-ylamide

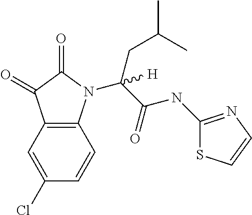

A stirred suspension of sodium hydride (159 mg) in N,N-dimethylformamide (15 mL) at 0° C. was treated with a solution of 5-chloro-1H-indole-2,3-dione (1.0 g) in N,N-dimethylformamide. The reaction mixture was stirred for 30 min at 0° C. and then 2-bromo-4-methyl-pentanoic acid methyl ester (prepared as in Example 9, 1.38 g) was added and it was stirred for 1 h at 0° C. It was then slowly allowed to warm to room temperature and stirred for another 3 h at room temperature. After this time, the reaction mixture was diluted with water and extracted with methylene chloride. The organic layers were combined and dried over sodium sulfate, filtered and the filterate concentrated in vacuo. The crude material was purified by column chromatography (silica gel) to afford 2-(5-chloro-2,3-dioxo-2,3-dihydro-indol-1-yl)-4-methyl-pentanoic acid methyl ester (1.0 g, 59%).

A mixture of 2-(5-chloro-2,3-dioxo-2,3-dihydro-indol-1-yl)-4-methyl-pentanoic acid methyl ester (1.0 g) in tetrahydrofuran:water (1:1, 20 mL) was stirred at room temperature and treated with lithium hydroxide monohydrate (272 mg). The reaction mixture was then stirred for 2 h at room temperature. After this time, the reaction mixture was concentrated in vacuo to remove the tetrahydrofuran. The remaining aqueous layer was diluted with water and then acidified to pH=2 with a 1N aqueous hydrochloric acid solution. The aqueous layer was then extracted with ethyl acetate and the organic layers combined, dried over sodium sulfate, filtered and the filterate concentrated in vacuo to afford 2-(5-chloro-2,3-dioxo-2,3-dihydro-indol-1-yl)-4-methyl-pentanoic acid (900 mg, 94%).

A mixture of 2-(5-chloro-2,3-dioxo-2,3-dihydro-indol-1-yl)-4-methyl-pentanoic acid (100 mg) and thiazol-2-ylamine (41 mg) in N,N-dimethylformamide (2 mL) at room temperature was treated with N,N-diisopropylethyl amine (190 μL) and (benzotriazol-1-yloxytris(dimethylamino)phosphonium hexafluorophosphate (225 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(5-chloro-2,3-dioxo-2,3-dihydro-indol-1-yl)-4-methyl-pentanoic acid thiazol-2-ylamide (70 mg, 61%) as an orange solid: LCMS ESI calcd for C₁₇H₁₆ClN₃O₃S (M+H)⁺ 378, observed 378.

### Example 102

### 2-(5-Chloro-2,3-dioxo-2,3-dihydro-indol-1-yl)-4-methyl-pentanoic acid pyridin-2-ylamide

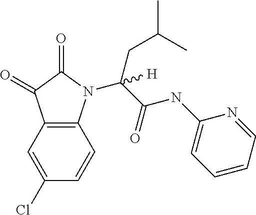

A mixture of 2-(5-chloro-2,3-dioxo-2,3-dihydro-indol-1-yl)-4-methyl-pentanoic acid (prepared as in Example 101, 100 mg) and pyridin-2-ylamine (38 mg) in N,N-dimethylformamide (2 mL) at room temperature was treated with N,N-diisopropylethyl amine (190 μL) and (benzotriazol-1-yloxytris(dimethylamino)phosphonium hexafluorophosphate (225 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(5-chloro-2,3-dioxo-2,3-dihydro-indol-1-yl)-4-methyl-pentanoic acid pyridin-2-ylamide (100 mg, 80%) as an orange solid: LCMS ESI calcd for C₁₉H₁₈ClN₃O₃ (M+H)⁺ 372, observed 372.

### Example 103

### 2-(5-Chloro-2,3-dioxo-2,3-dihydro-indol-1-yl)-4-methyl-pentanoic acid pyrazin-2-ylamide

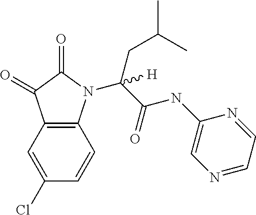

A mixture of 2-(5-chloro-2,3-dioxo-2,3-dihydro-indol-1-yl)-4-methyl-pentanoic acid (prepared as in Example 101, 100 mg) in methylene chloride (5 mL) and N,N-dimethylformamide (one drop) at room temperature was treated with oxalyl chloride (76 μL) and stirred for 30 min at room temperature. After this time the reaction mixture was concentrated in vacuo. The residue was dissolved in tetrahydrofuran (10 mL) and then treated with pyrazin-2-ylamine (37 mg) and 2,6-lutidine (120 μL). The resulting reaction mixture was then stirred overnight at room temperature. After this time, the reaction mixture was diluted with ethyl acetate, and washed with a 1N aqueous hydrochloric acid solution, saturated aqueous sodium bicarbonate solution and brine. The organic layers were dried over sodium sulfate, filtered and the filterate concentrated in vacuo. The crude material was purified by column chromatography (silica gel) to afford 2-(5-chloro-2,3-dioxo-2,3-dihydro-indol-1-yl)-4-methyl-pentanoic acid pyrazin-2-ylamide (50 mg, 40%) as an orange solid: LCMS ESI calcd for C₁₈H₁₇ClN₄O₃ (M+H)⁺ 373, observed 373.

### Example 104

### 2-(5-Chloro-2,3-dioxo-2,3-dihydro-indol-1-yl)-4-methyl-pentanoic acid (1-methyl-1H-pyrazol-3-yl)-amide

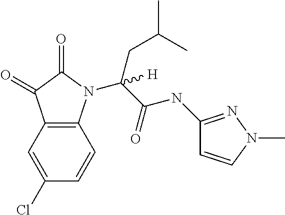

A mixture of 2-(5-chloro-2,3-dioxo-2,3-dihydro-indol-1-yl)-4-methyl-pentanoic acid (prepared as in Example 101, 100 mg) and 1-methyl-1H-pyrazol-3-ylamine (40 mg) in N,N-dimethylformamide (5 mL) at room temperature was treated with N,N-diisopropylethyl amine (200 μL) and (benzotriazol-1-yloxytris(dimethylamino)phosphonium hexafluorophosphate (225 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(5-chloro-2,3-dioxo-2,3-dihydro-indol-1-yl)-4-methyl-pentanoic acid (1-methyl-1H-pyrazol-3-yl)-amide (80 mg, 63%) as an orange solid: LCMS ESI calcd for C₁₈H₁₉ClN₄O₃ (M+H)⁺ 375, observed 375.

### Example 105

### 2-(5-Chloro-2-oxo-2,3-dihydro-indol-1-yl)-4-methyl-pentanoic acid thiazol-2-ylamide

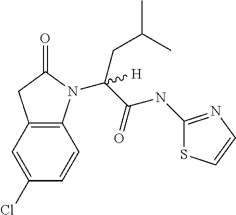

A mixture of 2-(5-chloro-2,3-dioxo-2,3-dihydro-indol-1-yl)-4-methyl-pentanoic acid (prepared as in Example 101, 100 mg) and hydrazine hydrate (50 μL) were placed in a sealed tube and heated at 120° C. for 15 min. The reaction mixture was then cooled to room temperature and concentrated in vacuo. The residue was dissolved in methylene chloride (30 mL) and washed with a 0.5N aqueous hydrochloric acid solution (2×10 mL) and a saturated aqueous brine solution (10 mL). The organic layer was then dried over sodium sulfate, filtered and the filterate concentrated in vacuo to afford 2-(5-chloro-2-oxo-2,3-dihydro-indol-1-yl)-4-methyl-pentanoic acid (80 mg, 84%).

A mixture of 2-(5-chloro-2-oxo-2,3-dihydro-indol-1-yl)-4-methyl-pentanoic acid (60 mg) and benzene (3 mL) was treated with thionyl chloride (19 μL) and heated at 80° C. for 2 h. After this time, the mixture was concentrated in vacuo to remove the benzene and the residue was dissolved in tetrahydrofuran (3 mL). The resulting solution was then treated with thiazol-2-ylamine (26 mg) and 2,6-lutidine (75 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(5-chloro-2-oxo-2,3-dihydro-indol-1-yl)-4-methyl-pentanoic acid thiazol-2-ylamide (27 mg, 34%) as a white solid: LCMS ESI calcd for C₁₇H₁₈lN₃O₂S (M+H)⁺ 364, observed 364.

### Example 106

### 2-(5-Chloro-2-oxo-2,3-dihydro-indol-1-yl)-4-methyl-pentanoic acid pyrazin-2-ylamide

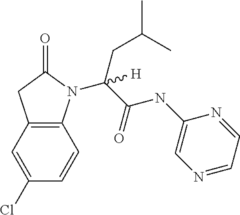

A mixture of 2-(5-chloro-2-oxo-2,3-dihydro-indol-1-yl)-4-methyl-pentanoic acid (prepared as in Example 105, 60 mg) and benzene (3 mL) was treated with thionyl chloride (19 μL) and heated at 80° C. for 2 h. After this time, the mixture was concentrated in vacuo to remove the benzene and the residue was dissolved in tetrahydrofuran (3 mL). The resulting solution was then treated with pyrazin-2-ylamine (24 mg) and 2,6-lutidine (75 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(5-chloro-2-oxo-2,3-dihydro-indol-1-yl)-4-methyl-pentanoic acid pyrazin-2-ylamide (7 mg, 10%) as a brown solid: LCMS ESI calcd for C₁₈H₁₉ClN₄O₂ (M+H)⁺ 359, observed 359.

### Example 107

### 2-(5-Chloro-2-oxo-2,3-dihydro-indol-1-yl)-4-methyl-pentanoic acid (1-methyl-1H-pyrazol-3-yl)-amide

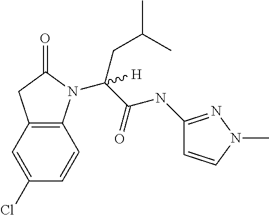

A mixture of 2-(5-chloro-2-oxo-2,3-dihydro-indol-1-yl)-4-methyl-pentanoic acid (prepared as in Example 105, 75 mg) and benzene (3 mL) were treated with thionyl chloride (64 μL) and heated at 80° C. for 30 min. After this time, the mixture was concentrated in vacuo to remove the benzene and the residue was dissolved in tetrahydrofuran (3 mL). The resulting solution was then treated with 1-methyl-1H-pyrazol-3-ylamine (31 mg) and 2,6-lutidine (81 μL) and stirred at room temperature overnight. The reaction mixture was then concentrated in vacuo to remove the tetrahydrofuran and the residue was dissolved in ethyl acetate and washed with a saturated aqueous brine solution. The organic layer was then dried over sodium sulfate, filtered and the filterate concentrated in vacuo and the residue was purified by column chromatography (silica gel) to afford 2-(5-chloro-2-oxo-2,3-dihydro-indol-1-yl)-4-methyl-pentanoic acid (1-methyl-1H-pyrazol-3-yl)-amide (15 mg, 5%): LCMS ESI calcd for C₁₈H₂₁ClN₄O₂ (M+H)⁺ 361, observed 361.

### Example 108

### 2-(5-Chloro-2-oxo-2,3-dihydro-indol-1-yl)-4-methyl-pentanoic acid pyridin-2-ylamide

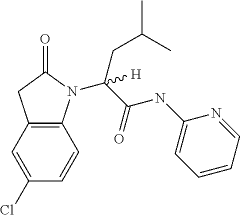

A mixture of 2-(5-chloro-2-oxo-2,3-dihydro-indol-1-yl)-4-methyl-pentanoic acid (prepared as in Example 105, 90 mg) and benzene (5 mL) was treated with thionyl chloride (28 μL) and heated at 80° C. for 2 h. After this time, the mixture was concentrated in vacuo to remove the benzene and the residue was dissolved in tetrahydrofuran (5 mL). The resulting solution was then treated with pyridin-2-ylamine (36 mg) and 2,6-lutidine (112 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(5-chloro-2-oxo-2,3-dihydro-indol-1-yl)-4-methyl-pentanoic acid pyridin-2-ylamide (55 mg, 48%): LCMS ESI calcd for C₁₉H₂₀ClN₃O₂ (M+H)⁺ 358, observed 358.

### Example 109

### 2-(6-Chloro-2,3-dioxo-2,3-dihydro-indol-1-yl)-4-methyl-pentanoic acid thiazol-2-ylamide

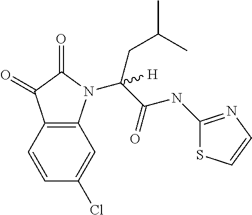

A mixture of sodium hydride (60% in oil, 264 mg) in N,N-dimethylformamide at 0° C. was treated with a solution of 6-chloro-1H-indole-2,3-dione (1.0 g) in N,N-dimethyl formamide (total volume is 25 mL). The reaction mixture was then stirred for 30 min at 0° C. After this time, the reaction mixture was treated with 2-bromo-4-methyl-pentanoic acid methyl ester (prepared as in Example 9, 1.38 g) and stirred for another 1 h at 0° C. The reaction mixture was then allowed to warm to room temperature and stirred for 3 h. After this time, the reaction was diluted with water and extracted with methylene chloride. The organic layers were combined and then dried over sodium sulfate, filtered to remove the drying agent and the filterate concentrated in vacuo. The residue obtained was then purified by silica gel column chromatography to afford 2-(6-chloro-2,3-dioxo-2,3-dihydro-indol-1-yl)-4-methyl-pentanoic acid methyl ester (1.3 g, 76%).

A mixture of 2-(6-chloro-2,3-dioxo-2,3-dihydro-indol-1-yl)-4-methyl-pentanoic acid methyl ester (1.3 g) in tetrahydrofuran:water (1:1, 20 mL) was treated with lithium hydroxide monohydrate (530 mg). The resulting reaction mixture was then stirred for 18 h at room temperature. The reaction mixture was then concentrated in vacuo to remove the tetrahydrofuran and the resulting aqueous layer was then dilute with water and acidified with a 1 N aqueous hydrochloric acid solution to pH=2. The aqueous layer was then extracted with ethyl acetate (3×20 mL). The organic layers were then combined and dried over sodium sulfate, filtered to remove the drying agent and the filterate concentrated in vacuo to afford 2-(6-chloro-2,3-dioxo-2,3-dihydro-indol-1-yl)-4-methyl-pentanoic acid (1.2 g, 97%).

A mixture of 2-(6-chloro-2,3-dioxo-2,3-dihydro-indol-1-yl)-4-methyl-pentanoic acid (75 mg) and thiazol-2-ylamine (31 mg) in N,N-dimethylformamide at room temperature was treated with N,N-diisopropylethyl amine (73 μL) and (benzotriazol-1-yloxytris(dimethylamino)phosphonium hexafluorophosphate (169 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(6-chloro-2,3-dioxo-2,3-dihydro-indol-1-yl)-4-methyl-pentanoic acid thiazol-2-ylamide (70 mg, 73%) as a yellow solid: LCMS ESI calcd for C₁₇H₁₆ClN₃O₃S (M+H)⁺ 378, observed 378.

### Example 110

### 2-(6-Chloro-2,3-dioxo-2,3-dihydro-indol-1-yl)-4-methyl-pentanoic acid pyridin-2-ylamide

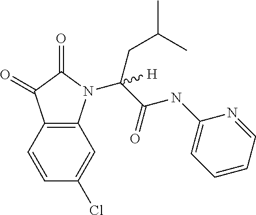

A mixture of 2-(6-chloro-2,3-dioxo-2,3-dihydro-indol-1-yl)-4-methyl-pentanoic acid (prepared as in Example 109, 75 mg) and pyridin-2-ylamine (29 mg) in N,N-dimethylformamide at room temperature was treated with N,N-diisopropylethyl amine (73 μL) and (benzotriazol-1-yloxytris(dimethylamino)phosphonium hexafluorophosphate (169 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(6-chloro-2,3-dioxo-2,3-dihydro-indol-1-yl)-4-methyl-pentanoic acid pyridin-2-ylamide (7 mg, 7%) as a yellow solid: LCMS ESI calcd for C₁₉H₁₈ClN₃O₃ (M+H)⁺ 372, observed 372.

### Example 111

### 2-(6-Chloro-2,3-dioxo-2,3-dihydro-indol-1-yl)-4-methyl-pentanoic acid (1-methyl-1H-pyrazol-3-yl)-amide

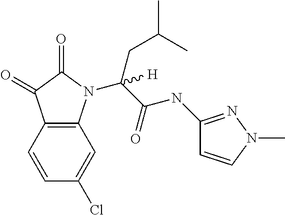

A mixture of 2-(6-chloro-2,3-dioxo-2,3-dihydro-indol-1-yl)-4-methyl-pentanoic acid (prepared as in Example 109, 75 mg) and 1-methyl-1H-pyrazol-3-ylamine (30 mg) in N,N-dimethylformamide at room temperature was treated with N,N-diisopropylethyl amine (73 μL) and (benzotriazol-1-yloxytris(dimethylamino)phosphonium hexafluorophosphate (169 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(6-chloro-2,3-dioxo-2,3-dihydro-indol-1-yl)-4-methyl-pentanoic acid (1-methyl-1H-pyrazol-3-yl)-amide (45 mg, 47%) as a yellow solid: LCMS ESI calcd for C₁₈H₁₉ClN₄O₃ (M+H)⁺ 375, observed 375.

### Example 112

### 2-(6-Chloro-2,3-dioxo-2,3-dihydro-indol-1-yl)-4-methyl-pentanoic acid pyrazin-2-ylamide

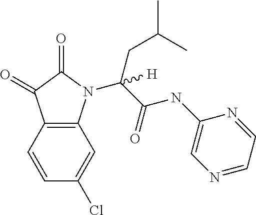

A mixture of 2-(6-chloro-2,3-dioxo-2,3-dihydro-indol-1-yl)-4-methyl-pentanoic acid (prepared as in Example 109, 100 mg) in methylene chloride (5 mL) was cooled to 0° C. and treated with oxalyl chloride (35 μL) followed by N,N-dimethylformamide (one drop) and stirred for 30 min at 0° C. After this time the reaction mixture was concentrated in vacuo. The residue was dissolved in tetrahydrofuran (5 mL) and then treated with pyrazin-2-ylamine (39 mg) and 2,6-lutidine (118 μL). The resulting reaction mixture was then stirred at room temperature for 16 h. After this time, the reaction mixture was concentrated in vacuo and the residue dissolved in ethyl acetate (10 mL), and washed with a 0.5N aqueous hydrochloric acid solution (2×3 mL), saturated aqueous sodium bicarbonate solution (2×3 mL) and brine (4 mL). The organic layers were dried over sodium sulfate, filtered and the filterate concentrated in vacuo. The crude material was purified by Biotage column chromatography (silica gel, 15% ethyl acetate/hexanes) to afford 2-(6-chloro-2,3-dioxo-2,3-dihydro-indol-1-yl)-4-methyl-pentanoic acid pyrazin-2-ylamide (52 mg, 41%): LCMS ESI calcd for C₁₈H₁₇ClN₄O₃ (M+H)⁺ 373, observed 373.

### Example 113

### 2-(6-Chloro-2-oxo-2,3-dihydro-indol-1-yl)-4-methyl-pentanoic acid pyrazin-2-ylamide

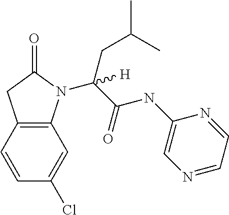

A mixture of 2-(6-chloro-2,3-dioxo-2,3-dihydro-indol-1-yl)-4-methyl-pentanoic acid (prepared as in Example 109, 500 mg) and hydrazine hydrate (1 mL) were placed in a sealed tube and heated at 120° C. for 30 min. The reaction mixture was then cooled to room temperature and concentrated in vacuo. The residue was dissolved in methylene chloride (15 mL) and washed with a 0.5N aqueous hydrochloric acid solution (2×7 mL) and a saturated aqueous brine solution (5 mL). The organic layer was then dried over sodium sulfate, filtered and the filterate concentrated in vacuo to afford 2-(6-chloro-2-oxo-2,3-dihydro-indol-1-yl)-4-methyl-pentanoic acid (420 mg, 88%).

A mixture of 2-(6-chloro-2-oxo-2,3-dihydro-indol-1-yl)-4-methyl-pentanoic acid (60 mg) and benzene (3 mL) was treated with thionyl chloride (19 μL) and heated at 80° C. for 2 h. After this time, the mixture was concentrated in vacuo to remove the benzene and the residue was dissolved in tetrahydrofuran (3 mL). The resulting solution was then treated with pyrazin-2-ylamine (24 mg) and 2,6-lutidine (75 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(6-chloro-2-oxo-2,3-dihydro-indol-1-yl)-4-methyl-pentanoic acid pyrazin-2-ylamide (35 mg, 46%) as a red solid: LCMS ESI calcd for C₁₈H₁₉ClN₄O₂ (M+H)⁺ 359, observed 359.

### Example 114

### 2-(6-Chloro-2-oxo-2,3-dihydro-indol-1-yl)-4-methyl-pentanoic acid pyridin-2-ylamide

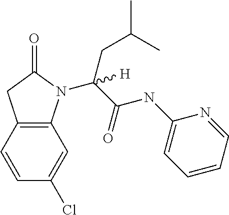

A mixture of 2-(6-chloro-2-oxo-2,3-dihydro-indol-1-yl)-4-methyl-pentanoic acid (prepared as in Example 113, 60 mg) and benzene (3 mL) was treated with thionyl chloride (19 μL) and heated at 80° C. for 2 h. After this time, the mixture was concentrated in vacuo to remove the benzene and the residue was dissolved in tetrahydrofuran (3 mL). The resulting solution was then treated with pyridin-2-ylamine (24 mg) and 2,6-lutidine (75 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(6-chloro-2-oxo-2,3-dihydro-indol-1-yl)-4-methyl-pentanoic acid pyridin-2-ylamide (20 mg, 26%) as an orange oil: LCMS ESI calcd for C₁₉H₂₀ClN₃O₂ (M+H)⁺ 358, observed 358.

### Example 115

### 2-(6-Chloro-2-oxo-2,3-dihydro-indol-1-yl)-4-methyl-pentanoic acid thiazol-2-ylamide

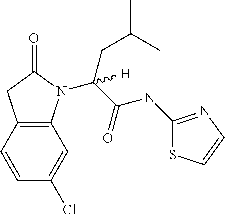

A mixture of 2-(6-chloro-2-oxo-2,3-dihydro-indol-1-yl)-4-methyl-pentanoic acid (prepared as in Example 113, 60 mg) and benzene (3 mL) was treated with thionyl chloride (19 μL) and heated at 80° C. for 2 h. After this time, the mixture was concentrated in vacuo to remove the benzene and the residue was dissolved in tetrahydrofuran (3 mL). The resulting solution was then treated with thiazol-2-ylamine (26 mg) and 2,6-lutidine (75 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(6-chloro-2-oxo-2,3-dihydro-indol-1-yl)-4-methyl-pentanoic acid thiazol-2-ylamide (56 mg, 72%) as a yellow solid: LCMS ESI calcd for C₁₇H₁₈ClN₃O₂S (M+H)⁺ 364, observed 364.

### Example 116

### 2-(6-Chloro-2-oxo-2,3-dihydro-indol-1-yl)-4-methyl-pentanoic acid (1-methyl-1H-pyrazol-3-yl)-amide

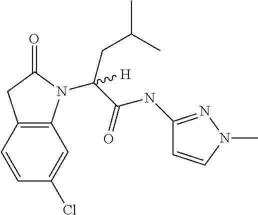

A mixture of 2-(6-chloro-2-oxo-2,3-dihydro-indol-1-yl)-4-methyl-pentanoic acid (prepared as in Example 113, 60 mg) and benzene (3 mL) was treated with thionyl chloride (19 μL) and heated at 80° C. for 2 h. After this time, the mixture was concentrated in vacuo to remove the benzene and the residue was dissolved in tetrahydrofuran (3 mL). The resulting solution was then treated with 1-methyl-1H-pyrazol-3-ylamine (25 mg) and 2,6-lutidine (75 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(6-chloro-2-oxo-2,3-dihydro-indol-1-yl)-4-methyl-pentanoic acid (1-methyl-1H-pyrazol-3-yl)-amide (47 mg, 60%) as a yellow solid: LCMS ESI calcd for C₁₈H₂₁ClN₄O₂ (M+H)⁺ 361, observed 361.

### Example 117

### 2-(6-Chloro-2,3-dioxo-2,3-dihydro-indol-1-yl)-3-cyclopentyl-N-thiazol-2-yl-propionamide

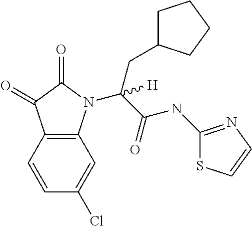

A mixture of sodium hydride (60% in oil, 264 mg) in N,N-dimethylformamide at 0° C. was treated with a solution of 6-chloro-1H-indole-2,3-dione (1.0 g) in N,N-dimethyl formamide (total volume is 25 mL). The reaction mixture was then stirred for 30 min at 0° C. After this time, the reaction mixture was treated with 2-bromo-3-cyclopentyl-propionic acid methyl ester (prepared as in Example 1, 1.55 g) and stirred for another 1 h at 0° C. The reaction mixture was then allowed to warm to room temperature and stirred for 3 h. After this time, the reaction was diluted with water and extracted with methylene chloride. The organic layers were combined and then dried over sodium sulfate, filtered to remove the drying agent and the filterate concentrated in vacuo. The residue obtained was then purified by silica gel column chromatography to afford 2-(6-chloro-2,3-dioxo-2,3-dihydro-indol-1-yl)-3-cyclopentyl-propionic acid methyl ester (1.4 g, 76%).

A mixture of 2-(6-chloro-2,3-dioxo-2,3-dihydro-indol-1-yl)-3-cyclopentyl-propionic acid methyl ester (1.4 g) in tetrahydrofuran:water (1:1, 20 mL) was treated with lithium hydroxide monohydrate (527 mg). The resulting reaction mixture was then stirred for 18 h at room temperature. The reaction mixture was then concentrated in vacuo to remove the tetrahydrofuran and the resulting aqueous layer was then dilute with water and acidified with a 1 N aqueous hydrochloric acid solution to pH=2. The aqueous layer was then extracted with ethyl acetate (3×20 mL). The organic layers were then combined and dried over sodium sulfate, filtered to remove the drying agent and the filterate concentrated in vacuo to afford 2-(6-chloro-2,3-dioxo-2,3-dihydro-indol-1-yl)-3-cyclopentyl-propionic acid (1.3 g, 97%).

A mixture of 2-(6-chloro-2,3-dioxo-2,3-dihydro-indol-1-yl)-3-cyclopentyl-propionic acid (75 mg) and thiazol-2-ylamine (28 mg) in N,N-dimethylformamide at room temperature was treated with N,N-diisopropylethyl amine (67 μL) and (benzotriazol-1-yloxytris(dimethylamino)phosphonium hexafluorophosphate (155 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(6-chloro-2,3-dioxo-2,3-dihydro-indol-1-yl)-3-cyclopentyl-N-thiazol-2-yl-propionamide (60 mg, 64%) as a yellow solid: LCMS ESI calcd for C₁₉H₁₈ClN₃O₃S (M+H)⁺ 404, observed 404.

### Example 118

### 2-(6-chloro-2,3-dioxo-2,3-dihydro-indol-1-yl)-3-cyclopentyl-N-pyridin-2-yl-propionamide

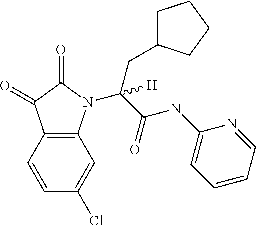

A mixture of 2-(6-chloro-2,3-dioxo-2,3-dihydro-indol-1-yl)-3-cyclopentyl-propionic acid (prepared as in Example 117, 75 mg) and pyridin-2-ylamine (26 mg) in N,N-dimethylformamide at room temperature was treated with N,N-diisopropylethyl amine (67 μL) and (benzotriazol-1-yloxytris(dimethylamino)phosphonium hexafluorophosphate (155 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(6-chloro-2,3-dioxo-2,3-dihydro-indol-1-yl)-3-cyclopentyl-N-pyridin-2-yl-propionamide (30 mg, 32%) as a yellow solid: LCMS ESI calcd for C₂₁H₂₀ClN₃O₃ (M+H)⁺ 398, observed 398.

### Example 119

### 2-(6-Chloro-2,3-dioxo-2,3-dihydro-indol-1-yl)-3-cyclopentyl-N-(1-methyl-1H-pyrazol-3-yl)-propionamide

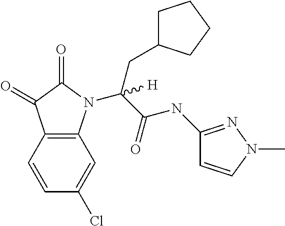

A mixture of 2-(6-chloro-2,3-dioxo-2,3-dihydro-indol-1-yl)-3-cyclopentyl-propionic acid (prepared as in Example 117, 75 mg) and 1-methyl-1H-pyrazol-3-ylamine (27 mg) in N,N-dimethylformamide at room temperature was treated with N,N-diisopropylethyl amine (67 μL) and (benzotriazol-1-yloxytris(dimethylamino)phosphonium hexafluorophosphate (155 mg) and stirred overnight. After this time, the reaction mixture was diluted with ethyl acetate and the organic layer was washed with a saturated aqueous ammonium chloride solution, saturated aqueous sodium bicarbonate solution, and brine. The organic layer was then dried over sodium sulfate, filtered and the filterate concentrated in vacuo. The crude material was then purified by column chromatography (silica gel) to afford 2-(6-chloro-2,3-dioxo-2,3-dihydro-indol-1-yl)-3-cyclopentyl-N-(1-methyl-1H-pyrazol-3-yl)-propionamide (55 mg, 59%) as a yellow solid: LCMS ESI calcd for C₂₀H₂₁ClN₄O₃ (M+H)⁺ 401, observed 401.

### Example 120

### 2-(6-Chloro-2,3-dioxo-2,3-dihydro-indol-1-yl)-3-cyclopentyl-N-pyrazin-2-yl-propionamide

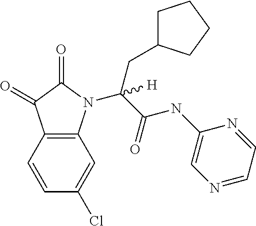

A mixture of 2-(6-chloro-2,3-dioxo-2,3-dihydro-indol-1-yl)-3-cyclopentyl-propionic acid (prepared as in Example 117, 75 mg) in methylene chloride (5 mL) was cooled to 0° C. and treated with oxalyl chloride (53 μL) followed by N,N-dimethylformamide (one drop) and stirred for 30 min at room temperature. After this time the reaction mixture was concentrated in vacuo. The residue was dissolved in tetrahydrofuran (5 mL) and then treated with pyrazin-2-ylamine (27 mg) and 2,6-lutidine (69 μL). The resulting reaction mixture was then stirred at room temperature overnight. After this time, the reaction mixture was diluted with ethyl acetate, and washed with a 1 N aqueous hydrochloric acid solution, saturated aqueous sodium bicarbonate solution and brine. The organic layers were dried over sodium sulfate, filtered and the filterate concentrated in vacuo. The crude material was purified by silica gel column chromatography to afford 2-(6-chloro-2,3-dioxo-2,3-dihydro-indol-1-yl)-3-cyclopentyl-N-pyrazin-2-yl-propionamide (45 mg, 48%): LCMS ESI calcd for C₂₀H₁₉ClN₄O₃ (M+H)⁺ 399, observed 399.

### Example 121

### 2-(6-Chloro-2-oxo-2,3-dihydro-indol-1-yl)-3-cyclopentyl-N-pyrazin-2-yl-propionamide

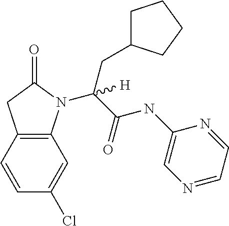

A mixture of 2-(6-chloro-2,3-dioxo-2,3-dihydro-indol-1-yl)-3-cyclopentyl-propionic acid (prepared as in Example 117, 400 mg) and hydrazine hydrate (0.5 mL) were placed in a sealed tube and heated at 120° C. for 15 min. The reaction mixture was then cooled to room temperature and concentrated in vacuo. The residue was dissolved in methylene chloride (30 mL) and washed with a 0.5N aqueous hydrochloric acid solution (2×10 mL) and a saturated aqueous brine solution (10 mL). The organic layer was then dried over sodium sulfate, filtered and the filterate concentrated in vacuo to afford 2-(6-chloro-2-oxo-2,3-dihydro-indol-1-yl)-3-cyclopentyl-propionic acid (360 mg, 94%).

A mixture of 2-(6-chloro-2-oxo-2,3-dihydro-indol-1-yl)-3-cyclopentyl-propionic acid (65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with pyrazin-2-ylamine (24 mg) and 2,6-lutidine (74 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(6-chloro-2-oxo-2,3-dihydro-indol-1-yl)-3-cyclopentyl-N-pyrazin-2-yl-propionamide (23 mg, 28%) as a brown solid: LCMS ESI calcd for C₂₀H₂₁ClN₄O₂ (M+H)⁺ 385, observed 385.

### Example 122

### 2-(6-Chloro-2-oxo-2,3-dihydro-indol-1-yl)-3-cyclopentyl-N-pyridin-2-yl-propionamide

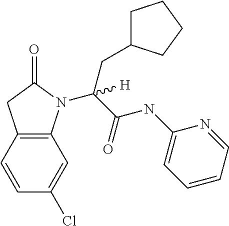

A mixture of 2-(6-chloro-2-oxo-2,3-dihydro-indol-1-yl)-3-cyclopentyl-propionic acid (prepared as in Example 121, 150 mg) and benzene (5 mL) was treated with thionyl chloride (43 μL) and heated at 80° C. for 2 h. After this time, the mixture was concentrated in vacuo to remove the benzene and the residue was dissolved in tetrahydrofuran (5 mL). The resulting solution was then treated with pyridin-2-ylamine (55 mg) and 2,6-lutidine (171 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(6-chloro-2-oxo-2,3-dihydro-indol-1-yl)-3-cyclopentyl-N-pyridin-2-yl-propionamide (24 mg, 13%): LCMS ESI calcd for C₂₁H₂₂ClN₃O₂ (M+H)⁺ 384, observed 384.

### Example 123

### 2-(6-Chloro-2-oxo-2,3-dihydro-indol-1-yl)-3-cyclopentyl-N-thiazol-2-yl-propionamide

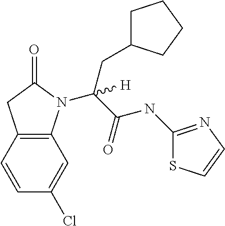

A mixture of 2-(6-chloro-2-oxo-2,3-dihydro-indol-1-yl)-3-cyclopentyl-propionic acid (prepared as in Example 121, 65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with thiazol-2-ylamine (25 mg) and 2,6-lutidine (74 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(6-chloro-2-oxo-2,3-dihydro-indol-1-yl)-3-cyclopentyl-N-thiazol-2-yl-propionamide (8 mg, 10%) as a yellow solid: LCMS ESI calcd for C₁₉H₂₀ClN₃O₂S (M+H)⁺ 390, observed 390.

### Example 124

### 2-(6-Chloro-2-oxo-2,3-dihydro-indol-1-yl)-3-cyclopentyl-N-(1-methyl-1H-pyrazol-3-yl)-propionamide

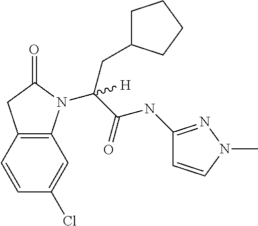

A mixture of 2-(6-chloro-2-oxo-2,3-dihydro-indol-1-yl)-4-methyl-pentanoic acid (prepared as in Example 121, 65 mg) and benzene (3 mL) was treated with thionyl chloride (18 μL) and heated at 80° C. for 2 h. After this time, the mixture was concentrated in vacuo to remove the benzene and the residue was dissolved in tetrahydrofuran (3 mL). The resulting solution was then treated with 1-methyl-1H-pyrazol-3-ylamine (25 mg) and 2,6-lutidine (74 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(6-chloro-2-oxo-2,3-dihydro-indol-1-yl)-3-cyclopentyl-N-(1-methyl-1H-pyrazol-3-yl)-propionamide (50 mg, 61%) as a brown solid: LCMS ESI calcd for C₂₀H₂₃ClN₄O₂ (M+H)⁺ 387, observed 387.

### Example 125

### 3-Cyclopentyl-2-(6-fluoro-2-oxo-2,3-dihydro-indol-1-yl)-N-thiazol-2-yl-propionamide

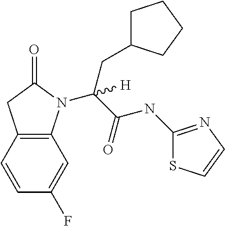

A stirred suspension of sodium hydride (60% dispersion in oil, 290 mg) in N,N-dimethylformamide at 0° C. was treated with a solution of 6-fluoro-1H-indole-2,3-dione (1.0 g) in N,N-dimethylformamide (total volume, 20 mL). The reaction mixture was stirred for 30 min at 0° C. and then 2-bromo-3-cyclopentyl-propionic acid methyl ester (prepared as in Example 1, 1.71 g) was added and it was stirred for 1 h at 0° C. It was then slowly allowed to warm to room temperature and stirred for another 3 h at room temperature. After this time, the reaction mixture was diluted with water and extracted with methylene chloride (3×10 mL). The organic layers were combined and dried over sodium sulfate, filtered and the filterate concentrated in vacuo. The crude material was purified by column chromatography (silica gel, 5% ethyl acetate/hexanes) to afford 3-cyclopentyl-2-(6-fluoro-2,3-dioxo-2,3-dihydro-indol-1-yl)-propionic acid methyl ester (1.0 g, 52%).

A mixture of 3-cyclopentyl-2-(6-fluoro-2,3-dioxo-2,3-dihydro-indol-1-yl)-propionic acid methyl ester (1.0 g) in tetrahydrofuran (10 mL) was treated with lithium hydroxide monohydrate (264 mg) in water (10 mL). The resulting reaction mixture was then stirred for 16 h at room temperature. The reaction mixture was then concentrated in vacuo to remove the tetrahydrofuran and the resulting aqueous layer was then dilute with water and acidified with a 5 N aqueous hydrochloric acid solution to pH=2. The aqueous layer was then extracted with ethyl acetate (2×10 mL). The organic layers were then combined washed with a saturated aqueous brine solution and dried over sodium sulfate, filtered to remove the drying agent and the filterate concentrated in vacuo to afford 3-cyclopentyl-2-(6-fluoro-2,3-dioxo-2,3-dihydro-indol-1-yl)-propionic acid (900 mg, 94%).

A mixture of 3-cyclopentyl-2-(6-fluoro-2,3-dioxo-2,3-dihydro-indol-1-yl)-propionic acid (900 mg) and hydrazine hydrate (2.0 mL) were placed in a sealed tube and heated at 120° C. for 15 min. The reaction mixture was then cooled to room temperature and concentrated in vacuo. The residue was dissolved in methylene chloride (30 mL) and washed with a 0.5N aqueous hydrochloric acid solution (2×10 mL) and a saturated aqueous brine solution (10 mL). The organic layer was then dried over sodium sulfate, filtered and the filterate concentrated in vacuo to afford 3-cyclopentyl-2-(6-fluoro-2-oxo-2,3-dihydro-indol-1-yl)-propionic acid (450 mg, 52%).

A mixture of 3-cyclopentyl-2-(6-fluoro-2-oxo-2,3-dihydro-indol-1-yl)-propionic acid (90 mg) and benzene (5 mL) was treated with thionyl chloride (27 μL) and heated at 80° C. for 2 h. After this time, the mixture was concentrated in vacuo to remove the benzene and the residue was dissolved in tetrahydrofuran (5 mL). The resulting solution was then treated with thiazol-2-ylamine (37 mg) and 2,6-lutidine (108 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(6-fluoro-2-oxo-2,3-dihydro-indol-1-yl)-N-thiazol-2-yl-propionamide (84 mg, 73%): LCMS ESI calcd for C₁₉H₂₀FN₃O₂S (M+H)⁺ 374, observed 374.

### Example 126

### 3-Cyclopentyl-2-(6-fluoro-2-oxo-2,3-dihydro-indol-1-yl)-N-(1-methyl-1H-pyrazol-3-yl)-propionamide

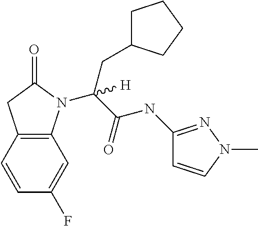

A mixture of 3-cyclopentyl-2-(6-fluoro-2-oxo-2,3-dihydro-indol-1-yl)-propionic acid (prepared as in Example 125, 90 mg) and benzene (5 mL) was treated with thionyl chloride (27 μL) and heated at 80° C. for 2 h. After this time, the mixture was concentrated in vacuo to remove the benzene and the residue was dissolved in tetrahydrofuran (5 mL). The resulting solution was then treated with 1-methyl-1H-pyrazol-3-ylamine (36 mg) and 2,6-lutidine (108 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(6-fluoro-2-oxo-2,3-dihydro-indol-1-yl)-N-(1-methyl-1H-pyrazol-3-yl)-propionamide (30 mg, 26%): LCMS ESI calcd for C₂₀H₂₃FN₄O₂ (M+H)⁺ 371, observed 371.

### Example 127

### 3-Cyclopentyl-2-(6-fluoro-2-oxo-2,3-dihydro-indol-1-yl)-N-pyridin-2-yl-propionamide

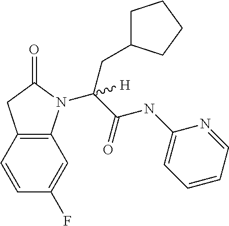

A mixture of 3-cyclopentyl-2-(6-fluoro-2-oxo-2,3-dihydro-indol-1-yl)-propionic acid (prepared as in Example 125, 90 mg) and benzene (5 mL) was treated with thionyl chloride (27 μL) and heated at 80° C. for 2 h. After this time, the mixture was concentrated in vacuo to remove the benzene and the residue was dissolved in tetrahydrofuran (5 mL). The resulting solution was then treated with pyridin-2-ylamine (35 mg) and 2,6-lutidine (108 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(6-fluoro-2-oxo-2,3-dihydro-indol-1-yl)-N-pyridin-2-yl-propionamide (89 mg, 79%): LCMS ESI calcd for C₂₁H₂₂FN₃O₂ (M+H)⁺ 368, observed 368.

### Example 128

### 3-Cyclopentyl-2-(6-fluoro-2-oxo-2,3-dihydro-indol-1-yl)-N-pyrazin-2-yl-propionamide

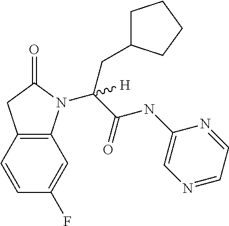

A mixture of 3-cyclopentyl-2-(6-fluoro-2-oxo-2,3-dihydro-indol-1-yl)-propionic acid (prepared as in Example 125, 90 mg) and benzene (5 mL) was treated with thionyl chloride (27 μL) and heated at 80° C. for 2 h. After this time, the mixture was concentrated in vacuo to remove the benzene and the residue was dissolved in tetrahydrofuran (5 mL). The resulting solution was then treated with pyrazin-2-ylamine (35 mg) and 2,6-lutidine (108 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(6-fluoro-2-oxo-2,3-dihydro-indol-1-yl)-N-pyrazin-2-yl-propionamide (26 mg, 23%): LCMS ESI calcd for C₂₀H₂₁FN₄O₂ (M+H)⁺ 369, observed 369.

### Example 129

### 3-Cyclopentyl-2-(6-methyl-2-oxo-2,3-dihydro-indol-1-yl)-N-thiazol-2-yl-propionamide

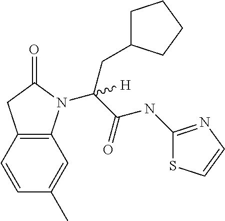

A stirred suspension of sodium hydride (60% dispersion in oil, 357 mg) in N,N-dimethylformamide at 0° C. was treated with a solution of 6-methyl-1H-indole-2,3-dione (1.5 g) in N,N-dimethylformamide (total volume, 20 mL). The reaction mixture was stirred for 30 min at 0° C. and then 2-bromo-3-cyclopentyl-propionic acid methyl ester (prepared as in Example 1, 2.1 g) was added and it was stirred for 1 h at 0° C. It was then slowly allowed to warm to room temperature and stirred for another 3 h at room temperature. After this time, the reaction mixture was diluted with water and extracted with methylene chloride (3×10 mL). The organic layers were combined and dried over sodium sulfate, filtered and the filterate concentrated in vacuo. The crude material was purified by column chromatography (silica gel, 5% ethyl acetate/hexanes) to afford 3-cyclopentyl-2-(6-methyl-2,3-dioxo-2,3-dihydro-indol-1-yl)-propionic acid methyl ester (700 mg, 30%).

A mixture of 3-cyclopentyl-2-(6-methyl-2,3-dioxo-2,3-dihydro-indol-1-yl)-propionic acid methyl ester (700 mg) in tetrahydrofuran (10 mL) was treated with lithium hydroxide monohydrate (187 mg) in water (10 mL). The resulting reaction mixture was then stirred for 16 h at room temperature. The reaction mixture was then concentrated in vacuo to remove the tetrahydrofuran and the resulting aqueous layer was then dilute with water and acidified with a 5 N aqueous hydrochloric acid solution to pH=2. The aqueous layer was then extracted with ethyl acetate (2×10 mL). The organic layers were then combined washed with a saturated aqueous brine solution and dried over sodium sulfate, filtered to remove the drying agent and the filterate concentrated in vacuo to afford 3-cyclopentyl-2-(6-methyl-2,3-dioxo-2,3-dihydro-indol-1-yl)-propionic acid (610 mg, 91%).

A mixture of 3-cyclopentyl-2-(6-methyl-2,3-dioxo-2,3-dihydro-indol-1-yl)-propionic acid (610 mg) and hydrazine hydrate (1.5 mL) were placed in a sealed tube and heated at 120° C. for 15 min. The reaction mixture was then cooled to room temperature and concentrated in vacuo. The residue was dissolved in methylene chloride (30 mL) and washed with a 0.5N aqueous hydrochloric acid solution (2×10 mL) and a saturated aqueous brine solution (10 mL). The organic layer was then dried over sodium sulfate, filtered and the filterate concentrated in vacuo to afford 3-cyclopentyl-2-(6-methyl-2-oxo-2,3-dihydro-indol-1-yl)-propionic acid (320 mg, 55%).

A mixture of 3-cyclopentyl-2-(6-methyl-2-oxo-2,3-dihydro-indol-1-yl)-propionic acid (90 mg) and benzene (5 mL) was treated with thionyl chloride (27 μL) and heated at 80° C. for 2 h. After this time, the mixture was concentrated in vacuo to remove the benzene and the residue was dissolved in tetrahydrofuran (5 mL). The resulting solution was then treated with thiazol-2-ylamine (38 mg) and 2,6-lutidine (110 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(6-methyl-2-oxo-2,3-dihydro-indol-1-yl)-N-thiazol-2-yl-propionamide (49 mg, 43%): LCMS ESI calcd for C₂₀H₂₃N₃O₂S (M+H)⁺ 370, observed 370.

### Example 130

### 3-Cyclopentyl-2-(6-methyl-2-oxo-2,3-dihydro-indol-1-yl)-N-pyrazin-2-yl-propionamide

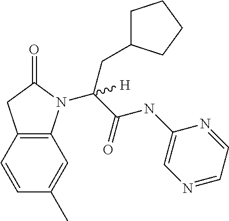

A mixture of 3-cyclopentyl-2-(6-methyl-2-oxo-2,3-dihydro-indol-1-yl)-propionic acid (prepared as in Example 129, 90 mg) and benzene (5 mL) was treated with thionyl chloride (27 μL) and heated at 80° C. for 2 h. After this time, the mixture was concentrated in vacuo to remove the benzene and the residue was dissolved in tetrahydrofuran (5 mL). The resulting solution was then treated with pyrazin-2-ylamine (36 mg) and 2,6-lutidine (110 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(6-methyl-2-oxo-2,3-dihydro-indol-1-yl)-N-pyrazin-2-yl-propionamide (34 mg, 30%): LCMS ESI calcd for C₂₁H₂₄N₄O₂ (M+H)⁺ 365, observed 365.

### Example 131

### 3-Cyclopentyl-2-(6-methyl-2-oxo-2,3-dihydro-indol-1-yl)-N-(1-methyl-1H-pyrazol-3-yl)-propionamide

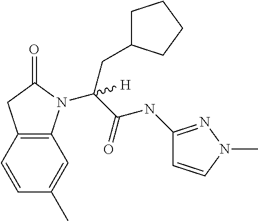

A mixture of 3-cyclopentyl-2-(6-methyl-2-oxo-2,3-dihydro-indol-1-yl)-propionic acid (prepared as in Example 129, 90 mg) and benzene (5 mL) was treated with thionyl chloride (27 μL) and heated at 80° C. for 2 h. After this time, the mixture was concentrated in vacuo to remove the benzene and the residue was dissolved in tetrahydrofuran (5 mL). The resulting solution was then treated with 1-methyl-1H-pyrazol-3-ylamine (37 mg) and 2,6-lutidine (110 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(6-methyl-2-oxo-2,3-dihydro-indol-1-yl)-N-(1-methyl-1H-pyrazol-3-yl)-propionamide (43 mg, 37%): LCMS ESI calcd for C₂₁H₂₆N₄O₂ (M+H)⁺ 367, observed 367.

### Example 132

### 3-Cyclopentyl-2-(6-methyl-2-oxo-2,3-dihydro-indol-1-yl)-N-pyridin-2-yl-propionamide

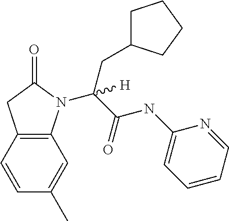

A mixture of 3-cyclopentyl-2-(6-methyl-2-oxo-2,3-dihydro-indol-1-yl)-propionic acid (prepared as in Example 129, 90 mg) and benzene (5 mL) was treated with thionyl chloride (27 μL) and heated at 80° C. for 2 h. After this time, the mixture was concentrated in vacuo to remove the benzene and the residue was dissolved in tetrahydrofuran (5 mL). The resulting solution was then treated with pyridin-2-ylamine (35 mg) and 2,6-lutidine (110 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 3-cyclopentyl-2-(6-methyl-2-oxo-2,3-dihydro-indol-1-yl)-N-pyridin-2-yl-propionamide (45 mg, 40%): LCMS ESI calcd for C₂₂H₂₅N₃O₂ (M+H)⁺ 364, observed 364.

### Example 133

### 2-(6-Bromo-2-oxo-2,3-dihydro-indol-1-yl)-3-cyclopentyl-N-pyrazin-2-yl-propionamide

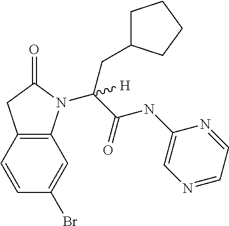

A stirred suspension of sodium hydride (60% dispersion in oil, 212 mg) in N,N-dimethylformamide at 0° C. was treated with a solution of 6-bromo-1H-indole-2,3-dione (1.0 g) in N,N-dimethylformamide (total volume, 20 mL). The reaction mixture was stirred for 30 min at 0° C. and then 2-bromo-3-cyclopentyl-propionic acid methyl ester (prepared as in Example 1, 1.25 g) was added and it was stirred for 1 h at 0° C. It was then slowly allowed to warm to room temperature and stirred for another 3 h at room temperature. After this time, the reaction mixture was diluted with water and extracted with methylene chloride (3×10 mL). The organic layers were combined and dried over sodium sulfate, filtered and the filterate concentrated in vacuo. The crude material was purified by column chromatography (silica gel, 5% ethyl acetate/hexanes) to afford 2-(6-bromo-2,3-dioxo-2,3-dihydro-indol-1-yl)-3-cyclopentyl-propionic acid methyl ester (1.0 g, 60%).

A mixture of 2-(6-bromo-2,3-dioxo-2,3-dihydro-indol-1-yl)-3-cyclopentyl-propionic acid methyl ester (1.0 g) in tetrahydrofuran (10 mL) was treated with lithium hydroxide monohydrate (221 mg) in water (10 mL). The resulting reaction mixture was then stirred for 16 h at room temperature. The reaction mixture was then concentrated in vacuo to remove the tetrahydrofuran and the resulting aqueous layer was then dilute with water and acidified with a 5 N aqueous hydrochloric acid solution to pH=2. The aqueous layer was then extracted with ethyl acetate (2×10 mL). The organic layers were then combined washed with a saturated aqueous brine solution and dried over sodium sulfate, filtered to remove the drying agent and the filterate concentrated in vacuo to afford 2-(6-bromo-2,3-dioxo-2,3-dihydro-indol-1-yl)-3-cyclopentyl-propionic acid (942 mg, 98%).

A mixture of 2-(6-bromo-2,3-dioxo-2,3-dihydro-indol-1-yl)-3-cyclopentyl-propionic acid (500 mg) and hydrazine hydrate (2 mL) were placed in a sealed tube and heated at 120° C. for 15 min. The reaction mixture was then cooled to room temperature and concentrated in vacuo. The residue was dissolved in methylene chloride (30 mL) and washed with a 0.5N aqueous hydrochloric acid solution (2×10 mL) and a saturated aqueous brine solution (10 mL). The organic layer was then dried over sodium sulfate, filtered and the filterate concentrated in vacuo to afford 2-(6-bromo-2-oxo-2,3-dihydro-indol-1-yl)-3-cyclopentyl-propionic acid (400 mg, 76%).

A mixture of 2-(6-bromo-2-oxo-2,3-dihydro-indol-1-yl)-3-cyclopentyl-propionic acid (105 mg) and benzene (5 mL) was treated with thionyl chloride (26 μL) and heated at 80° C. for 2 h. After this time, the mixture was concentrated in vacuo to remove the benzene and the residue was dissolved in tetrahydrofuran (5 mL). The resulting solution was then treated with pyrazin-2-ylamine (34 mg) and 2,6-lutidine (104 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(6-bromo-2-oxo-2,3-dihydro-indol-1-yl)-3-cyclopentyl-N-pyrazin-2-yl-propionamide (20 mg, 16%): LCMS ESI calcd for C₂₀H₂₁BrN₄O₂ (M+H)⁺ 429, observed 429.

### Example 134

### 2-(6-Bromo-2-oxo-2,3-dihydro-indol-1-yl)-3-cyclopentyl-N-thiazol-2-yl-propionamide

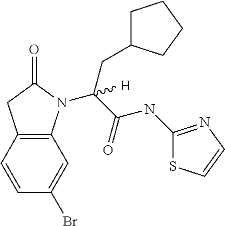

A mixture of 2-(6-bromo-2-oxo-2,3-dihydro-indol-1-yl)-3-cyclopentyl-propionic acid (prepared as in Example 133, 250 mg) and benzene (10 mL) was treated with thionyl chloride (62 μL) and heated at 80° C. for 2 h. After this time, the mixture was concentrated in vacuo to remove the benzene and the residue was dissolved in tetrahydrofuran (10 mL). The resulting solution was then treated with thiazol-2-ylamine (85 mg) and 2,6-lutidine (249 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(6-bromo-2-oxo-2,3-dihydro-indol-1-yl)-3-cyclopentyl-N-thiazol-2-yl-propionamide (150 mg, 49%): LCMS ESI calcd for C₁₉H₂₀BrN₃O₂S (M+H)⁺ 434, observed 434.

### Example 135

### 2-(6-Bromo-2-oxo-2,3-dihydro-indol-1-yl)-3-cyclopentyl-N-pyridin-2-yl-propionamide

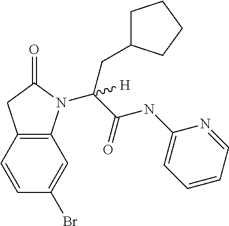

A mixture of 2-(6-bromo-2-oxo-2,3-dihydro-indol-1-yl)-3-cyclopentyl-propionic acid (prepared as in Example 133, 250 mg) and benzene (10 mL) was treated with thionyl chloride (62 μL) and heated at 80° C. for 2 h. After this time, the mixture was concentrated in vacuo to remove the benzene and the residue was dissolved in tetrahydrofuran (10 mL). The resulting solution was then treated with pyridin-2-ylamine (81 mg) and 2,6-lutidine (249 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(6-bromo-2-oxo-2,3-dihydro-indol-1-yl)-3-cyclopentyl-N-pyridin-2-yl-propionamide (140 mg, 46%): LCMS ESI calcd for C₂₁H₂₂BrN₃O₂ (M+H)⁺ 428, observed 428.

### Example 136

### 2-(6-Bromo-2-oxo-2,3-dihydro-indol-1-yl)-3-cyclopentyl-N-(1-methyl-1H-pyrazol-3-yl)-propionamide

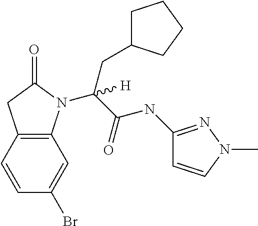

A mixture of 2-(6-bromo-2-oxo-2,3-dihydro-indol-1-yl)-3-cyclopentyl-propionic acid (prepared as in Example 133, 250 mg) and benzene (10 mL) was treated with thionyl chloride (62 μL) and heated at 80° C. for 2 h. After this time, the mixture was concentrated in vacuo to remove the benzene and the residue was dissolved in tetrahydrofuran (10 mL). The resulting solution was then treated with 1-methyl-1H-pyrazol-3-ylamine (83 mg) and 2,6-lutidine (249 μL) and stirred at room temperature for 16 h. The reaction mixture was then concentrated in vacuo to remove the tetrahydrofuran and the residue was dissolved in ethyl acetate and washed with a 1N aqueous hydrochloric acid solution. The organic layer was then dried over sodium sulfate, filtered and the filterate concentrated in vacuo and the residue was purified by Biotage column chromatography (silica gel) to afford 2-(6-bromo-2-oxo-2,3-dihydro-indol-1-yl)-3-cyclopentyl-N-(1-methyl-1H-pyrazol-3-yl)-propionamide (180 mg, 59%): LCMS ESI calcd for C₂₀H₂₃BrN₄O₂ (M+H)⁺ 431, observed 431.

### Example 137

### 2-(6-Cyano-2-oxo-2,3-dihydro-indol-1-yl)-3-cyclopentyl-N-pyridin-2-yl-propionamide

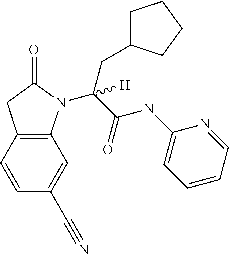

A mixture of 2-(6-bromo-2-oxo-2,3-dihydro-indol-1-yl)-3-cyclopentyl-N-pyridin-2-yl-propionamide (prepared as in Example 135, 100 mg) in a 1% water in N,N-dimethyl formamide solution (2.5 mL) was degassed for 10 min by bubbling argon through it. To this reaction mixture was then added zinc cyanide (31 mg), tris(dibenzylideneacetone)dipalladium(0) (32 mg) and 1,1′-bis(diphenyl phosphino)ferrocene (26 mg) and the reaction mixture was again degassed for a period of 10 min by bubbling argon through the reaction mixture. After this time, the reaction mixture was placed in a microwave reactor and heated at 150° C. for 15 min. After this time, the reaction mixture was diluted with water (10 mL) and ethyl acetate (10 mL). The organic layer was separated dried over sodium sulfate, filtered to remove the drying agent and the filterate concentrated in vacuo. The residue was purified by column chromatography (silica gel) to afford 2-(6-cyano-2-oxo-2,3-dihydro-indol-1-yl)-3-cyclopentyl-N-pyridin-2-yl-propionamide (11 mg, 11%): LCMS ESI calcd for C₂₂H₂₂N₄O₂ (M+H)⁺ 375, observed 375.

### Example 138

### 3-Cyclopentyl-2-(6-methanesulfonyl-2-oxo-2,3-dihydro-indol-1-yl)-N-thiazol-2-yl-propionamide

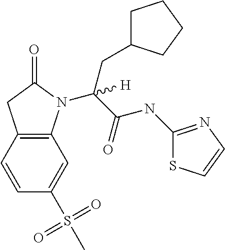

A mixture of 6-bromo-1H-indole-2,3-dione (500 mg) in N,N-dimethylformamide is treated with sodium methanthiolate (233 mg) and the mixture is heated at 70° C. for 16 h. After this time the reaction mixture is diluted with ethyl acetate (20 mL) and washed with water (3×10 mL), a saturated aqueous sodium chloride solution (10 mL) and the organic layer is dried over sodium sulfate, filtered to remove the drying agent and the filterated concentrated in vacuo to afford 6-methylsulfanyl-1H-indole-2,3-dione (230 mg, 54%).

A mixture of 6-methylsulfanyl-1H-indole-2,3-dione (230 mg) in N,N-dimethylformamide (5 mL) at 0° C. was treated with sodium hydride (60% dispersion in oil, 57 mg) and stirred for 30 min at 0° C. After this time, 2-bromo-3-cyclopentyl-propionic acid methyl ester (prepared as in Example 1, 336 mg) was added the reaction mixture was then allowed to warm to room temperature and stirred overnight. After this time, the reaction mixture was diluted with water (10 mL) and extracted with methylene chloride (2×5 mL). The combined organic layers were then washed with water (2×5 mL), a saturated aqueous sodium chloride solution and then dried over sodium sulfate, filtered and the filterate concentrated in vacuo. The crude material was purified by column chromatography (silica gel, 5% ethyl acetate/hexanes) to afford 3-cyclopentyl-2-(6-methylsulfanyl-2,3-dioxo-2,3-dihydro-indol-1-yl)-propionic acid methyl ester (250 mg, 61%).

A mixture of 3-cyclopentyl-2-(6-methylsulfanyl-2,3-dioxo-2,3-dihydro-indol-1-yl)-propionic acid methyl ester (250 mg) in tetrahydrofuran (5 mL) was treated with lithium hydroxide monohydrate (60 mg) in water (5 mL). The resulting reaction mixture was then stirred for 16 h at room temperature. The reaction mixture was then concentrated in vacuo to remove the tetrahydrofuran and the resulting aqueous layer was then dilute with water and acidified with a 5 N aqueous hydrochloric acid solution to pH=2. The aqueous layer was then extracted with ethyl acetate (2×3 mL). The organic layers were then combined washed with a saturated aqueous brine solution and dried over sodium sulfate, filtered to remove the drying agent and the filterate concentrated in vacuo to afford 3-cyclopentyl-2-(6-methylsulfanyl-2,3-dioxo-2,3-dihydro-indol-1-yl)-propionic acid (230 mg, 96%) and used in the next step without purification.

A mixture of 3-cyclopentyl-2-(6-methylsulfanyl-2,3-dioxo-2,3-dihydro-indol-1-yl)-propionic acid (230 mg) and hydrazine hydrate (1 mL) were placed in a sealed tube and heated at 120° C. for 15 min. The reaction mixture was then cooled to room temperature dilutued with methylene chloride (10 mL) and washed with a 1N aqueous hydrochloric acid solution (2×5 mL) and a saturated aqueous brine solution (5 mL). The organic layer was then dried over sodium sulfate, filtered and the filterate concentrated in vacuo. The residue was then purified by silica gel (60-120 mesh) column chromatography (30-50% ethyl acetate/hexanes) to afford 3-cyclopentyl-2-(6-methylsulfanyl-2-oxo-2,3-dihydro-indol-1-yl)-propionic acid (150 mg, 68%).

A mixture of 3-cyclopentyl-2-(6-methylsulfanyl-2-oxo-2,3-dihydro-indol-1-yl)-propionic acid (50 mg) and benzene (3 mL) was treated with thionyl chloride (14 μL) and heated at 80° C. for 2 h. After this time, the mixture was concentrated in vacuo to remove the benzene and the residue was dissolved in tetrahydrofuran (3 mL). The resulting solution was then cooled to 0° C. and treated with thiazol-2-ylamine (19 mg) and 2,6-lutidine (55 μL). The reaction mixture was then warmed to room temperature and stirred overnight. The reaction mixture was then concentrated in vacuo to remove the tetrahydrofuran and the residue was dissolved in ethyl acetate and washed with a 1N aqueous hydrochloric acid solution, a saturated aqueous sodium bicarbonate solution, and a saturated aqueous sodium chloride solution. The organic layer was then dried over sodium sulfate, filtered and the filterate concentrated in vacuo and the residue was purified by column chromatography (silica gel) to afford 3-cyclopentyl-2-(6-methylsulfanyl-2-oxo-2,3-dihydro-indol-1-yl)-N-thiazol-2-yl-propionamide (30 mg, 48%).

A solution of 3-cyclopentyl-2-(6-methylsulfanyl-2-oxo-2,3-dihydro-indol-1-yl)-N-thiazol-2-yl-propionamide (30 mg) in methanol (2 mL) was cooled to 0° C. and then treated with a mixture of OXONE (Potassium peroxymonosulfate) (138 mg) in water (2 mL). After this time, the reaction mixture was allowed to warm to room temperature and then stirred overnight. The reaction mixture was then concentrated in vacuo to remove the methanol and the crude was then dissolved in ethyl acetate (15 mL) and washed with water (2×5 mL) and a saturated aqueous sodium chloride solution (5 mL). The organic layer was then concentrated in vacuo. The crude material from this reaction was combined with a batch of crude material from a separate reaction run on twice the scale. The two batches of crude material were then purified by preparative HPLC to afford 3-cyclopentyl-2-(6-methanesulfonyl-2-oxo-2,3-dihydro-indol-1-yl)-N-thiazol-2-yl-propionamide (9.2 mg): LCMS ESI calcd for C₂₀H₂₃N₃O₄S₂ (M+H)⁺ 434, observed 434.

### Example 139

### In Vitro Glucokinase Activity

The compounds of formula I which include the compounds set forth in the Examples activated glucokinase in vitro by the procedure of this Example. In this manner, they increase the flux of glucose metabolism which causes increased insulin secretion. Therefore, the compounds of formula I are glucokinase activators useful for increasing insulin secretion.

Glucokinase In Vitro Assay Protocol: Glucokinase (GK) was assayed by coupling the production of glucose-6-phosphate to the generation of NADH with glucose-6-phosphate dehydrogenase (G6PDH, 0.75-1 kunits/mg; Boehringer Mannheim, Indianapolis, Ind.) from Leuconostoc mesenteroides as the coupling enzyme (Scheme 2).

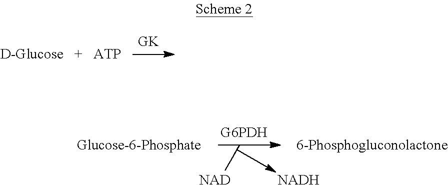

Recombinant human liver GK1 was expressed in E. coli as a glutathione S-transferase fusion protein (GST-GK) [Liang et al, 1995] and was purified by chromatography over a glutathione-Sepharose 4B affinity column using the procedure provided by the manufacturer (Amersham Pharmacia Biotech, Piscataway, N.J.). Previous studies have demonstrated that the enzymatic properties of native GK and GST-GK are essentially identical (Liang et al, 1995; Neet et al., 1990).

The assay was conducted at 30° C. in a flat bottom 96-well tissue culture plate from Costar (Cambridge, Mass.) with a final incubation volume of 120 μL. The incubation reaction contained the following: 25 mM Hepes buffer (pH 7.1), 25 mM KCl, 5 mM D-glucose, 1 mM ATP, 1.8 mM NAD, 2 mM MgCl₂, 1 μM sorbitol-6-phosphate, 1 mM dithiothreitol, test drug or 10% dimethylsulfoxide, ˜7 units/ml G6PDH, and GK (see below). All organic reagents were >98% pure and were from Boehringer Mannheim with the exceptions of D-glucose and Hepes which were from Sigma Chemical Co, St Louis, Mo. Test compounds were dissolved in dimethylsulfoxide and were added to the incubation reaction minus GST-GK in a volume of 12 μL to yield a final dimethylsulfoxide concentration of 10%. This mix was pre-incubated in the temperature controlled chamber of a SPECTRAmax 250 microplate spectrophotometer (Molecular Devices Corporation, Sunnyvale, Calif.) for 10 minutes to allow temperature equilibrium and then the reaction was started by the addition of 20 μL GST-GK.

After addition of enzyme, the increase in optical density (OD) at 340 nm was monitored spectrophotometrically to determine the rate of change (OD₃₄₀ per min). The GK activity (OD₃₄₀/min) in control wells (10% dimethylsulfoxide minus GK activators) was compared with the activity in wells containing test GK activators, and the concentration of activator that produced a 50% increase in the activity of GK, i.e., the SC_{1.5}, was calculated.

The table below provides the in vitro glucokinase activity for the compounds in the Examples:

| Example | | |
|---|---|---|
| number | SC_{1.5} | |
| 1 | 18.64 | μM |
| 2 | 5.93 | μM |
| 3 | 3.3 | μM |
| 4 | 5.21 | μM |
| 5 | 1.47 fold @ 30 uM | |
| 6 | 5.19 | μM |
| 7 | 17.55 | μM |
| 8 | 4.93 | μM |
| 9 | 1.33 fold @ 30 uM | |
| 10 | 1.20 fold @ 30 uM | |
| 11 | 16.22 | μM |
| 12 | 18.09 | μM |
| 13 | 6.94 | μM |
| 14 | 1.9 | μM |
| 15 | 6.5 | μM |
| 16 | 3.91 | μM |
| 17 | 1.39 fold @ 30 uM | |
| 18 | 13.42 | μM |
| 19 | 1.24 fold @ 30 uM | |
| 20 | 7.72 | μM |
| 21 | 1.23 | μM |
| 22 | 1.98 | μM |
| 23 | 7.4 | μM |
| 24 | 2.66 | μM |
| 25 | 16.78 | μM |
| 26 | 6.77 | μM |
| 27 | 1.39 fold @ 30 uM | |
| 28 | 9.95 | μM |
| 29 | 1.5 | μM |
| 30 | 0.33 | μM |
| 31 | 1.65 | μM |
| 32 | 0.76 | μM |
| 33 | 0.13 | μM |
| 34 | 1.07 | μM |
| 35 | 0.49 | μM |
| 36 | 0.63 | μM |
| 37 | 19.72 | μM |
| 38 | 2.57 | μM |
| 39 | 21.28 | μM |
| 40 | 5.97 | μM |
| 41 | 2.81 | μM |
| 42 | 8.12 | μM |
| 43 | 1.41 | μM |
| 44 | 3.67 | μM |
| 45 | 12.29 | μM |
| 46 | 2.32 | μM |
| 47 | 23.88 | μM |
| 48 | 5.48 | μM |
| 49 | 1.74 | μM |
| 50 | 8.39 | μM |
| 51 | 3.6 | μM |
| 52 | 4.76 | μM |
| 53 | 1.16 fold @ 30 uM | |
| 54 | 1.49 fold @ 30 uM | |
| 55 | 1.39 fold @ 30 uM | |
| 56 | 22.75 | μM |
| 57 | 13.89 | μM |
| 58 | 1.31 fold @ 30 uM | |
| 59 | 3.87 | μM |
| 60 | 5.48 | μM |
| 61 | 0.95 | μM |
| 62 | 6.18 | μM |
| 63 | 2.18 | μM |
| 64 | 0.48 | μM |
| 65 | 2.71 | μM |
| 66 | 1.69 | μM |
| 67 | 1.28 | μM |
| 68 | 1.32 fold @ 30 uM | |
| 69 | 7.43 | μM |
| 70 | 1.32 fold @ 30 uM | |
| 71 | 1.50 fold @ 30 uM | |
| 72 | 5.82 | μM |
| 73 | 17.96 | μM |
| 74 | 3.24 | μM |
| 75 | 11.4 | μM |
| 76 | 22.77 | μM |
| 77 | 6.67 | μM |
| 78 | 29.38 | μM |
| 79 | 16.22 | μM |
| 80 | 3.83 | μM |
| 81 | 11.29 | μM |
| 82 | 2.45 | μM |
| 83 | 4.47 | μM |
| 84 | 1.29 fold @ 30 uM | |
| 85 | 1.16 fold @ 30 uM | |
| 86 | 1.44 fold @ 30 uM | |
| 87 | 1.16 fold @ 30 uM | |
| 88 | 22.88 | μM |
| 89 | 1.23 fold @ 30 uM | |
| 90 | 12.84 | μM |
| 91 | 28.45 | μM |
| 92 | 3.01 | μM |
| 93 | 3.36 | μM |
| 94 | 1.04 | μM |
| 95 | 7.27 | μM |
| 96 | 4.04 | μM |
| 97 | 1.84 | μM |
| 98 | 1.22 | μM |
| 99 | 3.3 | μM |
| 100 | 0.49 | μM |
| 101 | 1.40 fold @ 30 uM | |
| 102 | 16.89 | μM |
| 103 | 1.30 fold @ 30 uM | |
| 104 | 1.21 fold @ 30 uM | |
| 105 | 6.74 | μM |
| 106 | 20.67 | μM |
| 107 | 1.22 fold @ 30 uM | |
| 108 | 4.95 | μM |
| 109 | 3.29 | μM |
| 110 | 1.49 | μM |
| 111 | 15.93 | μM |
| 112 | 4.85 | μM |
| 113 | 2.64 | μM |
| 114 | 0.91 | μM |
| 115 | 1.48 | μM |
| 116 | 7.27 | μM |
| 117 | 0.6 | μM |
| 118 | 0.23 | μM |
| 119 | 2.24 | μM |
| 120 | 0.9 | μM |
| 121 | 0.38 | μM |
| 122 | 0.1 | μM |
| 123 | 0.27 | μM |
| 124 | 1.1 | μM |
| 125 | 0.45 | μM |
| 126 | 1.86 | μM |
| 127 | 0.18 | μM |
| 128 | 0.81 | μM |
| 129 | 0.53 | μM |
| 130 | 0.61 | μM |
| 131 | 2.28 | μM |
| 132 | 0.23 | μM |
| 133 | 0.34 | μM |
| 134 | 0.19 | μM |
| 135 | 0.12 | μM |
| 136 | 0.88 | μM |
| 137 | 1.91 | μM |
| 138 | 2.94 | μM |

### REFERENCES

Liang, Y., Kesavan, P., Wang, L., Niswender, K., Tanizawa, Y., Permut, M. A., Magnuson, M., and Matschinsky, F. M. Variable effects of maturity-onset-diabetes-of-youth (MODY)-associated glucokinase mutations on the substrate interactions and stability of the enzyme. Biochem. J. 309: 167-173, 1995.
Neet, K., Keenan, R. P., and Tippett, P. S. Observation of a kinetic slow transition in monomeric glucokinase. Biochemistry 29; 770-777, 1990.

It is to be understood that the invention is not limited to the particular embodiments of the invention described above, as variations of the particular embodiments may be made and still fall within the scope of the appended claims.

What is claimed is:

1. A compound of formula (I): 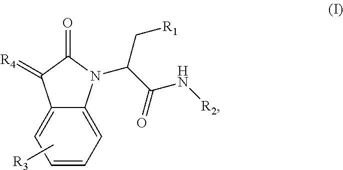
wherein:
R₁ is cycloalkyl or lower alkyl;
R₂ is an unsubstituted or substituted heteroaryl connected by a ring carbon atom to the amine group shown, with one heteroatom being nitrogen which is adjacent to the connecting ring carbon atom, said substituted heteroaryl being substituted at a position other than adjacent to said connecting carbon atom independently with lower alkyl;
R₃ is hydrogen, halogen, lower alkyl, alkoxy, halo-alkoxy, cyano or —SO₂CH₃; and
R₄ is oxygen or absent;
or a pharmaceutically acceptable salt thereof.

2. The compound according to claim 1, wherein R₁ is cycloalkyl.

3. The compound according to claim 1, wherein R₁ is cyclopentanyl.

4. The compound according to claim 1, wherein R₁ is lower alkyl.

5. The compound according to claim 1, wherein R₁ is isopropyl.

6. The compound according to claim 1, wherein R₂ is unsubstituted heteroaryl.

7. The compound according to claim 1, wherein R₂ is unsubstituted pyrazinyl, unsubstituted thiazolyl, unsubstituted pyrazolyl or unsubstituted pyridinyl.

8. The compound according to claim 1, wherein R₂ is heteroaryl substituted with lower alkyl.

9. The compound according to claim 1, wherein R₂ is substituted pyrazinyl, substituted thiazolyl, substituted pyrazolyl or substituted pyridinyl, wherein said substituent is methyl, 2-hydroxy-2-methyl propyl or 2,3-dihydroxy-propyl.

10. The compound according to claim 1, wherein R₃ is hydrogen or halogen.

11. The compound according to claim 1, wherein R₃ is lower alkyl, alkoxy or halo-alkoxy.

12. The compound according to claim 1, wherein R₃ is hydrogen, bromine, fluorine, chlorine, —OCH₃, —OCF₃, methyl, cyano or —SO₂CH₃.

13. The compound according to claim 1, wherein R₃ is hydrogen.

14. The compound according to claim 1, wherein R₃ is bromine, fluorine or chlorine.

15. The compound according to claim 1, wherein R₃ is methyl.

16. The compound according to claim 1, wherein R₃ is —OCH₃ or —OCF₃.

17. The compound according to claim 1, wherein R₃ is cyano or —SO₂CH₃.

18. The compound according to claim 1, wherein R₄ is oxygen.

19. The compound according to claim 1, wherein R₄ is absent.

20. The compound according to claim 1, wherein said compound is:
3-Cyclopentyl-2-(2,3-dioxo-2,3-dihydro-indol-1-yl)-N-pyrazin-2-yl-propionamide;
3-Cyclopentyl-2-(2-oxo-2,3-dihydro-indol-1-yl)-N-pyrazin-2-yl-propionamide;
3-Cyclopentyl-2-(2-oxo-2,3-dihydro-indol-1-yl)-N-thiazol-2-yl-propionamide;
3-Cyclopentyl-2-(2,3-dioxo-2,3-dihydro-indol-1-yl)-N-thiazol-2-yl-propionamide;
3-Cyclopentyl-2-(2,3-dioxo-2,3-dihydro-indol-1-yl)-N-(1-methyl-1H-pyrazol-3-yl)-propionamide;
3-Cyclopentyl-2-(2,3-dioxo-2,3-dihydro-indol-1-yl)-N-pyridin-2-yl-propionamide;
3-Cyclopentyl-N-(1-methyl-1H-pyrazol-3-yl)-2-(2-oxo-2,3-dihydro-indol-1-yl)-propionamide;
3-Cyclopentyl-2-(2-oxo-2,3-dihydro-indol-1-yl)-N-pyridin-2-yl-propionamide;
2-(2,3-Dioxo-2,3-dihydro-indol-1-yl)-4-methyl-pentanoic acid pyridin-2-ylamide;
2-(2,3-Dioxo-2,3-dihydro-indol-1-yl)-4-methyl-pentanoic acid thiazol-2-ylamide;
4-Methyl-2-(2-oxo-2,3-dihydro-indol-1-yl)-pentanoic acid thiazol-2-ylamide;
4-Methyl-2-(2-oxo-2,3-dihydro-indol-1-yl)-pentanoic acid pyridin-2-ylamide;
2-(4-Chloro-2,3-dioxo-2,3-dihydro-indol-1-yl)-3-cyclopentyl-N-pyrazin-2-yl-propionamide;
2-(4-Chloro-2,3-dioxo-2,3-dihydro-indol-1-yl)-3-cyclopentyl-N-pyridin-2-yl-propionamide;
2-(4-Chloro-2,3-dioxo-2,3-dihydro-indol-1-yl)-3-cyclopentyl-N-(1-methyl-1H-pyrazol-3-yl)-propionamide;
2-(4-Chloro-2,3-dioxo-2,3-dihydro-indol-1-yl)-3-cyclopentyl-N-thiazol-2-yl-propionamide;
2-(4-Chloro-2,3-dioxo-2,3-dihydro-indol-1-yl)-4-methyl-pentanoic acid pyrazin-2-ylamide;
2-(4-Chloro-2,3-dioxo-2,3-dihydro-indol-1-yl)-4-methyl-pentanoic acid pyridin-2-ylamide;
2-(4-Chloro-2,3-dioxo-2,3-dihydro-indol-1-yl)-4-methyl-pentanoic acid (1-methyl-1H-pyrazol-3-yl)-amide;
2-(4-Chloro-2,3-dioxo-2,3-dihydro-indol-1-yl)-4-methyl-pentanoic acid thiazol-2-ylamide;
2-(4-Chloro-2-oxo-2,3-dihydro-indol-1-yl)-3-cyclopentyl-N-pyridin-2-yl-propionamide;
2-(4-Chloro-2-oxo-2,3-dihydro-indol-1-yl)-3-cyclopentyl-N-thiazol-2-yl-propionamide;
2-(4-Chloro-2-oxo-2,3-dihydro-indol-1-yl)-3-cyc lop entyl-N-(1-methyl-1H-pyrazol-3-yl)-propionamide;
2-(4-Chloro-2-oxo-2,3-dihydro-indol-1-yl)-3-cyclopentyl-N-pyrazin-2-yl-propionamide;
2-(4-Chloro-2-oxo-2,3-dihydro-indol-1-yl)-4-methyl-pentanoic acid pyrazin-2-ylamide;
2-(4-Chloro-2-oxo-2,3-dihydro-indol-1-yl)-4-methyl-pentanoic acid pyridin-2-ylamide;
2-(4-Chloro-2-oxo-2,3-dihydro-indol-1-yl)-4-methyl-pentanoic acid (1-methyl-1H-pyrazol-3-yl)-amide;
2-(4-Chloro-2-oxo-2,3-dihydro-indol-1-yl)-4-methyl-pentanoic acid thiazol-2-ylamide;
3-Cyclopentyl-2-(2,3-dioxo-5-trifluoromethoxy-2,3-dihydro-indol-1-yl)-N-pyrazin-2-yl-propionamide;
3-Cyclopentyl-2-(2,3-dioxo-5-trifluoromethoxy-2,3-dihydro-indol-1-yl)-N-pyridin-2-yl-propionamide;
3-Cyclopentyl-2-(2,3-dioxo-5-trifluoromethoxy-2,3-dihydro-indol-1-yl)-N-(1-methyl-1H-pyrazol-3-yl)-propionamide;
3-Cyclopentyl-2-(2,3-dioxo-5-trifluoromethoxy-2,3-dihydro-indol-1-yl)-N-thiazol-2-yl-propionamide;
3-Cyclopentyl-2-(2-oxo-5-trifluoromethoxy-2,3-dihydro-indol-1-yl)-N-pyridin-2-yl-propionamide;
3-Cyclopentyl-N-(1-methyl-1H-pyrazol-3-yl)-2-(2-oxo-5-trifluoromethoxy-2,3-dihydro-indol-1-yl)-propionamide;
3-Cyclopentyl-2-(2-oxo-5-trifluoromethoxy-2,3-dihydro-indol-1-yl)-N-thiazol-2-yl-propionamide;
3-Cyclopentyl-2-(2-oxo-5-trifluoromethoxy-2,3-dihydro-indol-1-yl)-N-pyrazin-2-yl-propionamide;
2-(2,3-Dioxo-5-trifluoromethoxy-2,3-dihydro-indol-1-yl)-4-methyl-pentanoic acid pyrazin-2-ylamide;
2-(2,3-Dioxo-5-trifluoromethoxy-2,3-dihydro-indol-1-yl)-4-methyl-pentanoic acid pyridin-2-ylamide;
2-(2,3-Dioxo-5-trifluoromethoxy-2,3-dihydro-indol-1-yl)-4-methyl-pentanoic acid (1-methyl-1H-pyrazol-3-yl)-amide;
2-(2,3-Dioxo-5-trifluoromethoxy-2,3-dihydro-indol-1-yl)-4-methyl-pentanoic acid thiazol-2-ylamide;
4-Methyl-2-(2-oxo-5-trifluoromethoxy-2,3-dihydro-indol-1-yl)-pentanoic acid thiazol-2-ylamide;
4-Methyl-2-(2-oxo-5-trifluoromethoxy-2,3-dihydro-indol-1-yl)-pentanoic acid (1-methyl-1H-pyrazol-3-yl)-amide;
4-Methyl-2-(2-oxo-5-trifluoromethoxy-2,3-dihydro-indol-1-yl)-pentanoic acid pyridin-2-ylamide;
4-Methyl-2-(2-oxo-5-trifluoromethoxy-2,3-dihydro-indol-1-yl)-pentanoic acid pyrazin-2-ylamide;
3-Cyclopentyl-2-(5-methoxy-2,3-dioxo-2,3-dihydro-indol-1-yl)-N-pyrazin-2-yl-propionamide;
3-Cyclopentyl-2-(5-methoxy-2,3-dioxo-2,3-dihydro-indol-1-yl)-N-pyridin-2-yl-propionamide;
3-Cyclopentyl-2-(5-methoxy-2,3-dioxo-2,3-dihydro-indol-1-yl)-N-(1-methyl-1H-pyrazol-3-yl)-propionamide;
3-Cyclopentyl-2-(5-methoxy-2,3-dioxo-2,3-dihydro-indol-1-yl)-N-thiazol-2-yl-propionamide;
3-Cyclopentyl-2-(5-methoxy-2-oxo-2,3-dihydro-indol-1-yl)-N-pyridin-2-yl-propionamide;
3-Cyclopentyl-2-(5-methoxy-2-oxo-2,3-dihydro-indol-1-yl)-N-(1-methyl-1H-pyrazol-3-yl)-propionamide;
3-Cyclopentyl-2-(5-methoxy-2-oxo-2,3-dihydro-indol-1-yl)-N-thiazol-2-yl-propionamide;
3-Cyclopentyl-2-(5-methoxy-2-oxo-2,3-dihydro-indol-1-yl)-N-pyrazin-2-yl-propionamide;
2-(5-Methoxy-2,3-dioxo-2,3-dihydro-indol-1-yl)-4-methyl-pentanoic acid pyrazin-2-ylamide;
2-(5-Methoxy-2,3-dioxo-2,3-dihydro-indol-1-yl)-4-methyl-pentanoic acid pyridin-2-ylamide;
2-(5-Methoxy-2,3-dioxo-2,3-dihydro-indol-1-yl)-4-methyl-pentanoic acid thiazol-2-ylamide;
2-(5-Methoxy-2-oxo-2,3-dihydro-indol-1-yl)-4-methyl-pentanoic acid thiazol-2-ylamide;
2-(5-Methoxy-2-oxo-2,3-dihydro-indol-1-yl)-4-methyl-pentanoic acid pyridin-2-ylamide;
2-(5-Methoxy-2-oxo-2,3-dihydro-indol-1-yl)-4-methyl-pentanoic acid pyrazin-2-ylamide;
3-Cyclopentyl-N-[1-(2-hydroxy-2-methyl-propyl)-1H-pyrazol-3-yl]-2-(2-oxo-2,3-dihydro-indol-1-yl)-propionamide;
2-(5-Bromo-2,3-dioxo-2,3-dihydro-indol-1-yl)-3-cyclopentyl-N-pyrazin-2-yl-propionamide;
2-(5-Bromo-2,3-dioxo-2,3-dihydro-indol-1-yl)-3-cyclopentyl-N-pyridin-2-yl-propionamide;
2-(5-Bromo-2,3-dioxo-2,3-dihydro-indol-1-yl)-3-cyclopentyl-N-(1-methyl-1H-pyrazol-3-yl)-propionamide;
2-(5-Bromo-2,3-dioxo-2,3-dihydro-indol-1-yl)-3-cyclopentyl-N-thiazol-2-yl-propionamide;
2-(5-Bromo-2-oxo-2,3-dihydro-indol-1-yl)-3-cyclopentyl-N-pyridin-2-yl-propionamide;
2-(5-Bromo-2-oxo-2,3-dihydro-indol-1-yl)-3-cyclopentyl-N-(1-methyl-1H-pyrazol-3-yl)-propionamide;
2-(5-Bromo-2-oxo-2,3-dihydro-indol-1-yl)-3-cyclopentyl-N-thiazol-2-yl-propionamide;
2-(5-Bromo-2-oxo-2,3-dihydro-indol-1-yl)-3-cyclopentyl-N-pyrazin-2-yl-propionamide;
2-(5-Bromo-2,3-dioxo-2,3-dihydro-indol-1-yl)-4-methyl-pentanoic acid pyrazin-2-ylamide;
2-(5-Bromo-2,3-dioxo-2,3-dihydro-indol-1-yl)-4-methyl-pentanoic acid pyridin-2-ylamide;
2-(5-Bromo-2,3-dioxo-2,3-dihydro-indol-1-yl)-4-methyl-pentanoic acid (1-methyl-1H-pyrazol-3-yl)-amide;
2-(5-Bromo-2,3-dioxo-2,3-dihydro-indol-1-yl)-4-methyl-pentanoic acid thiazol-2-ylamide;
2-(5-Bromo-2-oxo-2,3-dihydro-indol-1-yl)-4-methyl-pentanoic acid thiazol-2-ylamide;
2-(5-Bromo-2-oxo-2,3-dihydro-indol-1-yl)-4-methyl-pentanoic acid (1-methyl-1H-pyrazol-3-yl)-amide;
2-(5-Bromo-2-oxo-2,3-dihydro-indol-1-yl)-4-methyl-pentanoic acid pyridin-2-ylamide;
2-(5-Bromo-2-oxo-2,3-dihydro-indol-1-yl)-4-methyl-pentanoic acid pyrazin-2-ylamide;
3-Cyclopentyl-2-(5-methyl-2,3-dioxo-2,3-dihydro-indol-1-yl)-N-pyrazin-2-yl-propionamide;
3-Cyclopentyl-2-(5-methyl-2,3-dioxo-2,3-dihydro-indol-1-yl)-N-pyridin-2-yl-propionamide;
3-Cyclopentyl-2-(5-methyl-2,3-dioxo-2,3-dihydro-indol-1-yl)-N-(1-methyl-1H-pyrazol-3-yl)-propionamide;
3-Cyclopentyl-2-(5-methyl-2,3-dioxo-2,3-dihydro-indol-1-yl)-N-thiazol-2-yl-propionamide;
3-Cyclopentyl-2-(5-methyl-2-oxo-2,3-dihydro-indol-1-yl)-N-pyridin-2-yl-propionamide;
3-Cyclopentyl-2-(5-methyl-2-oxo-2,3-dihydro-indol-1-yl)-N-(1-methyl-1H-pyrazol-3-yl)-propionamide;
3-Cyclopentyl-2-(5-methyl-2-oxo-2,3-dihydro-indol-1-yl)-N-thiazol-2-yl-propionamide;
3-Cyclopentyl-2-(5-methyl-2-oxo-2,3-dihydro-indol-1-yl)-N-pyrazin-2-yl-propionamide;
4-Methyl-2-(5-methyl-2,3-dioxo-2,3-dihydro-indol-1-yl)-pentanoic acid pyrazin-2-ylamide;
4-Methyl-2-(5-methyl-2,3-dioxo-2,3-dihydro-indol-1-yl)-pentanoic acid pyridin-2-ylamide;
4-Methyl-2-(5-methyl-2,3-dioxo-2,3-dihydro-indol-1-yl)-pentanoic acid (1-methyl-1H-pyrazol-3-yl)-amide;
4-Methyl-2-(5-methyl-2,3-dioxo-2,3-dihydro-indol-1-yl)-pentanoic acid thiazol-2-ylamide;
4-Methyl-2-(5-methyl-2-oxo-2,3-dihydro-indol-1-yl)-pentanoic acid thiazol-2-ylamide;
4-Methyl-2-(5-methyl-2-oxo-2,3-dihydro-indol-1-yl)-pentanoic acid (1-methyl-1H-pyrazol-3-yl)-amide;
4-Methyl-2-(5-methyl-2-oxo-2,3-dihydro-indol-1-yl)-pentanoic acid pyridin-2-ylamide;
4-Methyl-2-(5-methyl-2-oxo-2,3-dihydro-indol-1-yl)-pentanoic acid pyrazin-2-ylamide;
2-(5-Chloro-2,3-dioxo-2,3-dihydro-indol-1-yl)-3-cyclopentyl-N-thiazol-2-yl-propionamide;
2-(5-Chloro-2,3-dioxo-2,3-dihydro-indol-1-yl)-3-cyclopentyl-N-[1-((R)-2,3-dihydroxy-propyl)-1H-pyrazol-3-yl]-propionamide;
2-(5-Chloro-2,3-dioxo-2,3-dihydro-indol-1-yl)-3-cyclopentyl-N-pyridin-2-yl-propionamide;
2-(5-Chloro-2,3-dioxo-2,3-dihydro-indol-1-yl)-3-cyclopentyl-N-(1-methyl-1H-pyrazol-3-yl)-propionamide;
2-(5-Chloro-2,3-dioxo-2,3-dihydro-indol-1-yl)-3-cyclopentyl-N-pyrazin-2-yl-propionamide;
2-(5-Chloro-2-oxo-2,3-dihydro-indol-1-yl)-3-cyclopentyl-N-pyrazin-2-yl-propionamide;
2-(5-Chloro-2-oxo-2,3-dihydro-indol-1-yl)-3-cyclopentyl-N-thiazol-2-yl-propionamide;
2-(5-Chloro-2-oxo-2,3-dihydro-indol-1-yl)-3-cyclopentyl-N-(1-methyl-1H-pyrazol-3-yl)-propionamide;
2-(5-Chloro-2-oxo-2,3-dihydro-indol-1-yl)-3-cyclopentyl-N-pyridin-2-yl-propionamide;
2-(5-Chloro-2,3-dioxo-2,3-dihydro-indol-1-yl)-4-methyl-pentanoic acid thiazol-2-ylamide;
2-(5-Chloro-2,3-dioxo-2,3-dihydro-indol-1-yl)-4-methyl-pentanoic acid pyridin-2-ylamide;
2-(5-Chloro-2,3-dioxo-2,3-dihydro-indol-1-yl)-4-methyl-pentanoic acid pyrazin-2-ylamide;
2-(5-Chloro-2,3-dioxo-2,3-dihydro-indol-1-yl)-4-methyl-pentanoic acid (1-methyl-1H-pyrazol-3-yl)-amide;
2-(5-Chloro-2-oxo-2,3-dihydro-indol-1-yl)-4-methyl-pentanoic acid thiazol-2-ylamide;
2-(5-Chloro-2-oxo-2,3-dihydro-indol-1-yl)-4-methyl-pentanoic acid pyrazin-2-ylamide;
2-(5-Chloro-2-oxo-2,3-dihydro-indol-1-yl)-4-methyl-pentanoic acid (1-methyl-1H-pyrazol-3-yl)-amide;
2-(5-Chloro-2-oxo-2,3-dihydro-indol-1-yl)-4-methyl-pentanoic acid pyridin-2-ylamide;
2-(6-Chloro-2,3-dioxo-2,3-dihydro-indol-1-yl)-4-methyl-pentanoic acid thiazol-2-ylamide;
2-(6-Chloro-2,3-dioxo-2,3-dihydro-indol-1-yl)-4-methyl-pentanoic acid pyridin-2-ylamide;
2-(6-Chloro-2,3-dioxo-2,3-dihydro-indol-1-yl)-4-methyl-pentanoic acid (1-methyl-1H-pyrazol-3-yl)-amide;
2-(6-Chloro-2,3-dioxo-2,3-dihydro-indol-1-yl)-4-methyl-pentanoic acid pyrazin-2-ylamide;
2-(6-Chloro-2-oxo-2,3-dihydro-indol-1-yl)-4-methyl-pentanoic acid pyrazin-2-ylamide;
2-(6-Chloro-2-oxo-2,3-dihydro-indol-1-yl)-4-methyl-pentanoic acid pyridin-2-ylamide;
2-(6-Chloro-2-oxo-2,3-dihydro-indol-1-yl)-4-methyl-pentanoic acid thiazol-2-ylamide;
2-(6-Chloro-2-oxo-2,3-dihydro-indol-1-yl)-4-methyl-pentanoic acid (1-methyl-1H-pyrazol-3-yl)-amide;
2-(6-Chloro-2,3-dioxo-2,3-dihydro-indol-1-yl)-3-cyclopentyl-N-thiazol-2-yl-propionamide;
2-(6-chloro-2,3-dioxo-2,3-dihydro-indol-1-yl)-3-cyclopentyl-N-pyridin-2-yl-propionamide;
2-(6-Chloro-2,3-dioxo-2,3-dihydro-indol-1-yl)-3-cyclopentyl-N-(1-methyl-1H-pyrazol-3-yl)-propionamide;
2-(6-Chloro-2,3-dioxo-2,3-dihydro-indol-1-yl)-3-cyclopentyl-N-pyrazin-2-yl-propionamide;
2-(6-Chloro-2-oxo-2,3-dihydro-indol-1-yl)-3-cyclopentyl-N-pyrazin-2-yl-propionamide;
2-(6-Chloro-2-oxo-2,3-dihydro-indol-1-yl)-3-cyclopentyl-N-pyridin-2-yl-propionamide;
2-(6-Chloro-2-oxo-2,3-dihydro-indol-1-yl)-3-cyclopentyl-N-thiazol-2-yl-propionamide;
2-(6-Chloro-2-oxo-2,3-dihydro-indol-1-yl)-3-cyclopentyl-N-(1-methyl-1H-pyrazol-3-yl)-propionamide;
3-Cyclopentyl-2-(6-fluoro-2-oxo-2,3-dihydro-indol-1-yl)-N-thiazol-2-yl-propionamide;
3-Cyclopentyl-2-(6-fluoro-2-oxo-2,3-dihydro-indol-1-yl)-N-(1-methyl-1H-pyrazol-3-yl)-propionamide;
3-Cyclopentyl-2-(6-fluoro-2-oxo-2,3-dihydro-indol-1-yl)-N-pyridin-2-yl-propionamide;
3-Cyclopentyl-2-(6-fluoro-2-oxo-2,3-dihydro-indol-1-yl)-N-pyrazin-2-yl-propionamide;
3-Cyclopentyl-2-(6-methyl-2-oxo-2,3-dihydro-indol-1-yl)-N-thiazol-2-yl-propionamide;
3-Cyclopentyl-2-(6-methyl-2-oxo-2,3-dihydro-indol-1-yl)-N-pyrazin-2-yl-propionamide;
3-Cyclopentyl-2-(6-methyl-2-oxo-2,3-dihydro-indol-1-yl)-N-(1-methyl-1H-pyrazol-3-yl)-propionamide;
3-Cyclopentyl-2-(6-methyl-2-oxo-2,3-dihydro-indol-1-yl)-N-pyridin-2-yl-propionamide;
2-(6-Bromo-2-oxo-2,3-dihydro-indol-1-yl)-3-cyclopentyl-N-pyrazin-2-yl-propionamide;
2-(6-Bromo-2-oxo-2,3-dihydro-indol-1-yl)-3-cyclopentyl-N-thiazol-2-yl-propionamide;
2-(6-Bromo-2-oxo-2,3-dihydro-indol-1-yl)-3-cyclopentyl-N-pyridin-2-yl-propionamide;
2-(6-Bromo-2-oxo-2,3-dihydro-indol-1-yl)-3-cyclopentyl-N-(1-methyl-1H-pyrazol-3-yl)-propionamide;
2-(6-Cyano-2-oxo-2,3-dihydro-indol-1-yl)-3-cyclopentyl-N-pyridin-2-yl-propionamide; or
3-Cyclopentyl-2-(6-methanesulfonyl-2-oxo-2,3-dihydro-indol-1-yl)-N-thiazol-2-yl-propionamide.

21. A pharmaceutical composition comprising a therapeutically effective amount of a compound according to claim 1 and a therapeutically inert carrier.

22. A method for the treatment of diabetes, comprising the step of administering a therapeutically effective amount of a compound according to claim 1 to a patient in need thereof.
